(19) United States
(12) Patent Application Publication
(10) Pub. No.: US 2018/0021364 A1
(43) Pub. Date: Jan. 25, 2018

(54) CENTRAL NERVOUS SYSTEM TARGETING POLYNUCLEOTIDES

(71) Applicant: VOYAGER THERAPEUTICS, INC., CAMBRIDGE, MA (US)

(72) Inventors: Gregory Robert Stewart, Plymouth, MN (US); Dinah Wen-Yee Sah, Hopkinton, MA (US); Jinzhao Hou, Belmont, MA (US); Adrian Philip Kells, Arlington, MA (US); Martin Goulet, Weston, MA (US); Kartik Ramamoorthi, Pennington, NJ (US); Pengcheng Zhou, Lexington, MA (US); Yanqun Shu, Winchester, MA (US)

(21) Appl. No.: 15/543,773

(22) PCT Filed: Jan. 15, 2016
(86) PCT No.: PCT/US16/13671

§ 371 Date: Jul. 14, 2017

Related U.S. Application Data
(60) Provisional application No. 62/104,101, filed on Jan. 16, 2015.
(60) Provisional application No. 62/152,185, filed on Apr. 24, 2015.
(60) Provisional application No. 62/152,283, filed on Apr. 24, 2015.
(60) Provisional application No. 62/159,888, filed on May 11, 2015.
(60) Provisional application No. 62/159,882, filed on May 11, 2015.

(51) Int. Cl. A61K 31/7088; C07K 14/47; A61K 48/00

(52) U.S. Cl. CPC A61K 31/7088; A61K 48/005; A61K 48/0075; A61K 48/0083; C07K 14/47; C12N 2750/14143

(57) ABSTRACT

The invention relates to compositions and methods for the preparation, manufacture and therapeutic use of polynucleotides encoding payloads for the treatment of CNS disorders.

### CROSS REFERENCE TO RELATED APPLICATIONS

This application claims the benefit of U.S. Provisional Patent Application No. 62/104,101, entitled Methods of Delivery to the Central Nervous System, filed Jan. 16, 2015, U.S. Provisional Patent Application No. 62/152,185, entitled Methods of Delivery to the Central Nervous System, filed Apr. 24, 2015, U.S. Provisional Patent Application No. 62/152,283, entitled Central Nervous System Targeting Polynucleotides, filed Apr. 24, 2015, U.S. Provisional Patent Application No. 62/159,888, entitled Methods of Delivery to the Central Nervous System, filed May 11, 2015, U.S. Provisional Patent Application No. 62/159,882, entitled Central Nervous System Targeting Polynucleotides, filed May 11, 2015; the contents of each of which are herein incorporated by reference in their entirety.

### REFERENCE TO THE SEQUENCE LISTING

The present application is being filed along with a Sequence Listing in electronic format. The Sequence Listing is provided as a file entitled 1022PCT.txt created on Jan. 15, 2016 which is 635,539 bytes in size. The information in the electronic format of the sequence listing is incorporated herein by reference in its entirety.

### FIELD OF THE INVENTION

The present invention relates to compositions, methods and processes for the formulation and for the administration of a gene therapy agent using parvovirus e.g., adeno-associated virus (AAV) to the CNS, CNS tissues, CNS structures or CNS cells.

### BACKGROUND OF THE INVENTION

Use of adeno-associated virus (AAV) to deliver therapeutic agents (i.e., transgenes) to the central nervous system offers a means to achieve a widespread distribution of delivered genes in the CNS. Tissue of the CNS is highly heterogeneous and consists of different cell types including different types of neurons (e.g. excitatory and inhibitory neurons) and glial cells (e.g., oligodendrocytes, astrocytes and microglia). The characterization of different AAV capsid serotypes reveals that different AAV serotypes have different efficiency of transduction to different CNS tissues (e.g., cervical spinal cord and hippocampus) and cells (e.g., neurons or glial cells).

Studies, such as those referenced herein examining the targeting of specific tissues and cell types of the CNS by AAV capsids address one part of the problem of effective clinical treatment of CNS disorders by AAV delivery of therapeutic transgenes. The appropriate expression of the therapeutic transgene encoding the delivered payload, both temporally and spatially within the desired cell type, is critical to achieving the desired ameliorative effect. The properties of regulatory elements that drive expression of exogenous payloads from AAV genomes have not been well characterized.

On this background there remains, however, much work to be done to optimize delivery of therapeutic agents to the central nervous system. Better understanding and optimizing delivery parameters for viral particle distribution as described herein will lead to safer and more effective gene therapy. AAVs have emerged as one of the most widely studied and utilized viral particles for gene transfer to mammalian cells. See, e.g., Tratschin et al., Mol. Cell Biol., 5(11):3251-3260 (1985) and Grimm et al., Hum. Gene Ther., 10(15):2445-2450 (1999).

The present invention addresses the need for new technologies by providing AAV-based compositions and complexes which go beyond those of the art by providing for administration and/or delivery of recombinant adeno-associated viral particles in the treatment of diseases or disorders of the CNS, CNS tissues and/or CNS structures.

While delivery is exemplified in the AAV context, other viral vectors, non-viral vectors, nanoparticles, or liposomes may be similarly used to deliver the therapeutic transgenes and include, but are not limited to, vector genomes of any of the AAV serotypes or other parvoviral viral delivery vehicles or lentivirus, etc. The observations and teachings extend to any macromolecular structure, including modified cells, introduced into the CNS in the manner as described herein.

### SUMMARY OF THE INVENTION

The present invention provides AAV capsid serotypes with specific CNS cell type tropism, expression levels and bio-distribution in the CNS. Additionally, the present invention provides regulatory elements and codon optimization of the AAV genome useful in vitro and in vivo in both cell lines and primary CNS cell types. Accordingly, the present invention provides novel AAV particles with novel combinations of capsid and/or payload that target specific cells and/or tissue in a particular anatomic location in the CNS.

Described herein are compositions, methods, processes, kits and devices for the design, preparation, manufacture and/or formulation of AAV particles. In some embodiments, payloads, such as but not limited to AAV polynucleotides, may be encoded by payload constructs or contained within plasmids or vectors or recombinant adeno-associated viruses (AAVs).

The present invention also provides administration and/or delivery methods for vectors and viral particles, e.g., AAV particles, for the treatment or amelioration of diseases or disorders of the CNS. Such methods may involve the inhibition of gene expression, gene replacement or gene activation. Such outcomes are achieved by utilizing the methods and compositions taught herein.

The details of various embodiments of the invention are set forth in the description below. Other features, objects, and advantages of the invention will be apparent from the description and the drawings, and from the claims.

### BRIEF DESCRIPTION OF THE DRAWINGS

FIG. 1A-1D shows slab 1 and slab 2 individual and average frataxin expression on day 28 in mice treated via intracerebroventricular (ICV) or intracisternal (CM) administration.

FIG. 2A-2H shows frataxin mRNA expression levels in the CNS and other peripheral organs in tested monkeys after intrathecal (IT) L1 bolus dosing of each capsids.

FIG. 3A-3F shows frataxin expression profiles by the capsid serotypes across rostro-caudal spinal cord segments and dorsal root ganglia (DRG) in monkeys.

FIG. 4A-4B shows frataxin mRNA expression in dentate nucleus and liver for all tested capsids in monkeys.

### DETAILED DESCRIPTION OF THE INVENTION

Viruses of the Parvoviridae family are small non-enveloped icosahedral capsid viruses characterized by a single stranded DNA genome. Parvoviridae family viruses consist of two subfamilies: Parvovirinae, which infect vertebrates, and Densovirinae, which infect invertebrates. This virus family is used as a biological tool due to a relatively simple structure that may be easily manipulated with standard molecular biology techniques. The genome of the virus may be modified to contain a minimum of components for the assembly of a functional recombinant virus, or viral particle, which is loaded with or engineered to express or deliver a desired nucleic acid construct or payload, e.g., a transgene, polypeptide-encoding polynucleotide or modulatory nucleic acid, which may be delivered to a target cell, tissue or organism.

The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which is incorporated by reference in its entirety.

The Parvoviridae family comprises the Dependovirus genus which includes adeno-associated viruses (AAVs) capable of replication in vertebrate hosts including, but not limited to, human, primate, bovine, canine, equine, and ovine species.

The details of one or more embodiments of the invention are set forth in the accompanying description below. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

Parvovirus, e.g. AAV Particle Payload: Transgenes, Polypeptide-Encoding Polynucleotides and/or Modulatory Nucleic Acids

In some embodiments, the present invention provides administration and/or delivery methods for vectors and viral particles, e.g., AAV particles, for the treatment and/or amelioration of diseases or disorders of the CNS (e.g., Friedreich's ataxia).

AAV particles of the present invention may comprise a nucleic acid sequence encoding at least one “payload.” As used herein, a “payload” or “payload construct” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid.

The payload may comprise any nucleic acid known in the art which is useful for modulating the expression (by supplementation or gene replacement or by inhibition using a modulatory nucleic acid) in a target cell transduced or contacted with the AAV particle carrying the payload.

The payload construct may comprise a combination of coding and non-coding nucleic acid sequences.

In one embodiment, the nucleic acid sequence of the AAV particle may be a payload construct.

In one embodiment, the payload construct encodes more than one payload. As a non-limiting example, a payload construct encoding more than one payload may be replicated and packaged into a viral particle. A target cell transduced with a viral particle comprising more than one payload may express each of the payloads in a single cell.

In some embodiments, the payload construct may encode a coding or non-coding RNA.

In one embodiment, the payload is a polypeptide which may be a peptide or protein. A protein encoded by the payload construct may comprise a secreted protein, an intracellular protein, an extracellular protein, and/or a membrane protein. The encoded proteins may be structural or functional. Proteins encoded by the payload construct include, but are not limited to, mammalian proteins. The AAV particles described herein may be useful in the fields of human disease, antibodies, viruses, veterinary applications and a variety of in vivo and in vitro settings.

In some embodiments, AAV particles described herein are useful in the field of medicine for the treatment, palliation and/or amelioration of conditions or diseases such as, but not limited to, blood, cardiovascular, CNS, and/or genetic disorders.

In some embodiments, AAV particles in accordance with the present invention may be used for the treatment of disorders, and/or conditions, including but not limited to, neurological disorders (e.g. Alzheimer's disease, Huntington's disease; autism; Parkinson's disease; Spinal muscular atrophy, Friedreich's ataxia). In one embodiment, the payload encodes a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ, or ex vivo.

The components of an mRNA include, but are not limited to, a coding region, a 5′UTR, a 3′UTR, a 5′ cap and a poly-A tail. In some embodiments, the encoded mRNA or any portion of the AAV genome may be codon optimized.

In one embodiment the protein encoded by the payload construct is between 50-5000 amino acids in length. In some embodiments the protein encoded is between 50-2000 amino acids in length. In some embodiments the protein encoded is between 50-1000 amino acids in length. In some embodiments the protein encoded is between 50-1500 amino acids in length. In some embodiments the protein encoded is between 50-1000 amino acids in length. In some embodiments the protein encoded is between 50-800 amino acids in length. In some embodiments the protein encoded is between 50-600 amino acids in length. In some embodiments the protein encoded is between 50-400 amino acids in length. In some embodiments the protein encoded is between 50-200 amino acids in length. In some embodiments the protein encoded is between 50-100 amino acids in length.

In some embodiments the peptide encoded by the payload construct is between 4-50 amino acids in length. In one embodiment, the shortest length of a region of the payload of the present invention encoding a peptide can be the length that is sufficient to encode for a tetrapeptide, a pentapeptide, a hexapeptide, a heptapeptide, an octapeptide, a nonapeptide, or a decapeptide. In another embodiment, the length may be sufficient to encode a peptide of 2-30 amino acids, e.g. 5-30, 10-30, 2-25, 5-25, 10-25, or 10-20 amino acids. The length may be sufficient to encode for a peptide of at least 11, 12, 13, 14, 15, 17, 20, 25 or 30 amino acids, or a peptide that is no longer than 50 amino acids, e.g. no longer than 35, 30, 25, 20, 17, 15, 14, 13, 12, 11 or 10 amino acids.

In one embodiment, an AAV particle comprises at least one polynucleotide, e.g., viral genome, encoding at least one payload.

In one embodiment, the AAV particle comprises an AAV capsid selected from the groups consisting of AAV1, AAV2, AAV3, AAV4, AAV5, AAV6, AAV7, AAV8, AAV9, AAV10, and AAV11, AAV12, AAVrh8, AAVrh10, AAV-DJ, and AAV-DJ/8 capsid serotypes, or variants thereof (e.g., AAV3A and AAV3B).

In one embodiment, the serotype which may be useful in the present invention may be AAVDJ8 (or AAV-DJ8). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

### Modulatory Nucleic Acids

An RNA encoded by the viral genome or DNA of the payload construct may also comprise a tRNA, rRNA, tmRNA, miRNA, RNAi, siRNA, piRNA, shRNA antisense RNA, double stranded RNA, snRNA, snoRNA, and long non-coding RNA (ncRNA). Examples of such lncRNA molecules and RNAi constructs designed to target such lncRNA any of which may be encoded in the payload constructs are taught in International Publication, WO2012/018881 A2, the content of which is incorporated by reference in their entirety.

In one embodiment, the payload construct encodes a microRNA or miRNA, or engineered precursors thereof, as the payload. These payloads along with siRNA, shRNA, antisense molecules and the like may also be referred to as “modulatory nucleic acid” payloads.

MicroRNAs (or miRNA) are 19-25 nucleotide long noncoding RNAs that bind to the 3′UTR of nucleic acid molecules and down-regulate gene expression either by reducing nucleic acid molecule stability or by inhibiting translation. The payload constructs described herein may encode one or more microRNA target sequences, microRNA sequences, or microRNA seeds, or any known precursors thereof such as pre- or pri-microRNAs. Such sequences may correspond to any known microRNA such as those taught in US Publication US2005/0261218 and US Publication US2005/0059005, the content of which is incorporated herein by reference in their entirety.

A microRNA sequence comprises a “seed” region, i.e., a sequence in the region of positions 2-8 of the mature microRNA, which sequence has perfect Watson-Crick complementarity to the miRNA target sequence. A microRNA seed may comprise positions 2-8 or 2-7 of the mature microRNA. In some embodiments, a microRNA seed may comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may comprise 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K K, Johnston W K, Garrett-Engele P, Lim L P, Bartel D P; Mol Cell. 2007 Jul. 6; 27(1):91-105; each of which is herein incorporated by reference in their entirety. The bases of the microRNA seed have complete complementarity with the target sequence.

When designed to inhibit or silence a gene, the AAV particles comprising payload constructs of the present invention may also encode a payload which may be processed to produce a siRNA, miRNA or other double stranded (ds) or single stranded (ss) gene modulatory nucleic acids or motifs.

Accordingly, the encoded siRNA duplexes or dsRNA can be used to inhibit gene expression in a cell, in particular cells of the CNS. In some aspects, the inhibition of gene expression refers to an inhibition by at least about 20%, preferably by at least about 30%, 40%, 50%, 60%, 70%, 80%, 85%, 90%, 95% and 100%. Accordingly, the protein product of the targeted gene may be inhibited by at least about 20%, preferably by at least about 30%, 40%, 50%, 60%, 70%, 80%, 85%, 90%, 95% and 100%. The gene can be either a wild type gene or a mutated gene with at least one mutation. Accordingly, the targeted protein is either wild type protein or a mutated protein with at least one mutation.

In some embodiments, the present invention provides methods for treating, or ameliorating a disease or condition associated with abnormal gene and/or protein in a subject in need of treatment, the method comprising administering to the subject any effective amount of at least one AAV particle encoding an siRNA duplex targeting the gene, delivering duplex into targeted cells, inhibiting the gene expression and protein production, and ameliorating symptoms of the disease or condition in the subject.

### Gene Replacement or Activation

When designed to increase the expression of a gene or replace a gene, AAV particles may comprise a viral genome comprising a payload construct which encodes a normal gene to replace a mutated, defective or nonfunctional copy of that gene in the recipient.

### Functional Payloads

In one embodiment, a payload may comprise polypeptides that serve as marker proteins to assess cell transformation and expression, fusion proteins, polypeptides having a desired biological activity, gene products that can complement a genetic defect, RNA molecules, transcription factors, and other gene products that are of interest in regulation and/or expression. In another embodiment, a payload may comprise nucleotide sequences that provide a desired effect or regulatory function (e.g., transposons, transcription factors).

The encoded payload may comprise a gene therapy product. A gene therapy product may include, but is not limited to, a polypeptide, RNA molecule, or other gene product that, when expressed in a target cell, provides a desired therapeutic effect. In some embodiments, a gene therapy product may comprise a substitute for a non-functional gene that is absent or mutated.

A payload construct encoding a payload may comprise or encode a selectable marker. A selectable marker may comprise a gene sequence or a protein encoded by a gene sequence expressed in a host cell that allows for the identification, selection, and/or purification of the host cell from a population of cells that may or may not express the selectable marker. In one embodiment, the selectable marker provides resistance to survive a selection process that would otherwise kill the host cell, such as treatment with an antibiotic. In another embodiment, an antibiotic selectable marker may comprise one or more antibiotic resistance factors, including but not limited to neomycin resistance (e.g., neo), hygromycin resistance, kanamycin resistance, and/or puromycin resistance.

In some embodiments, any nucleic acid sequence encoding a polypeptide can be used as a selectable marker comprising recognition by a specific antibody.

In some embodiments, a payload construct encoding a payload may comprise a selectable marker including, but not limited to, β-lactamase, luciferase, β-galactosidase, or any other reporter gene as that term is understood in the art, including cell-surface markers, such as CD4 or the truncated nerve growth factor (NGFR) (for GFP, see WO 96/23810; Heim et al., Current Biology 2:178-182 (1996); Heim et al., Proc. Natl. Acad. Sci. USA (1995); or Heim et al., Science 373:663-664 (1995); for β-lactamase, see WO 96/30540); the contents of each of which are herein incorporated by reference in their entirety.

In some embodiments, a payload construct encoding a selectable marker may comprise a fluorescent protein. A fluorescent protein as herein described may comprise any fluorescent marker including but not limited to green, yellow, and/or red fluorescent protein (GFP, YFP, and/or RFP).

In accordance with the invention, a payload comprising a nucleic acid for expression of a payload in a target cell will be incorporated into the viral genome and produced located between two ITR sequences, or on either side of an asymmetrical ITR engineered with two D regions.

A payload construct encoding one or more payloads for expression in a target cell may comprise one or more payload or non-payload nucleotide sequences operably linked to at least one target cell-compatible promoter. A person skilled in the art may recognize that a target cell may require a specific promoter including but not limited to a promoter that is species specific, inducible, tissue-specific, or cell cycle-specific Parr et al., Nat. Med. 3:1145-9 (1997).

### Promoters

A person skilled in the art may recognize that expression in a target cell may require a specific promoter, including but not limited to, a promoter that is species specific, inducible, tissue-specific, or cell cycle-specific (Parr et al., Nat. Med. 3:1145-9 (1997); the contents of which are herein incorporated by reference in its entirety).

In one embodiment, the promoter is deemed to be efficient for expression of the payload encoded in the viral genome of the AAV particle.

In one embodiment, the promoter is a promoter deemed to be efficient for driving expression in the cell being targeted.

In one embodiment, the promoter provides expression of a payload for a period of time in targeted tissues such as, but not limited to, nervous system tissues. Expression from a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years. In one embodiment, the promoter is a weak promoter for sustained expression of a payload in nervous tissues. In one embodiment, the promoter is a weak promoter for sustained frataxin expression in nervous system tissue such as, but not limited to, neuronal tissue and glial tissue.

In one embodiment, the Friedreich's ataxia (FRDA) promoter is used in the viral genomes of the AAV particles described herein.

In one embodiment, there is a region located approximately ˜5 kb upstream of the first exon of the encoded payload, more specifically, there is a 17 bp region located approximately 4.9 kb upstream of the first exon of the encoded fraxtaxin gene in order to allow for expression with the FRDA promoter (See e.g., Puspasari et al. Long Range Regulation of Human FXN Gene Expression, PLOS ONE, 2011; the contents of which is herein incorporated by reference in its entirety).

In one embodiment, the promoter may be a promoter which is less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800.

In one embodiment, the promoter may be a combination of two or more components of the same or different promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. In one embodiment, the promoter is a combination of a 382 nucleotide CMV-enhancer sequence and a 260 nucleotide CBA-promoter sequence.

In one embodiment, the payload construct of the viral genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety). Non-limiting examples of elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

Promoters which promote expression in most tissues include, but are not limited to, human elongation factor 1α-subunit (EF1α), immediate-early cytomegalovirus (CMV), chicken β-actin (CBA) and its derivative CAG, the β glucuronidase (GUSB), or ubiquitin C (UBC). Tissue-specific expression elements can be used to restrict expression to certain cell types such as, but not limited to, nervous system promoters which can be used to restrict expression to neurons, astrocytes, or oligodendrocytes. Non-limiting example of tissue-specific expression elements for neurons include neuron-specific enolase (NSE), platelet-derived growth factor (PDGF), platelet-derived growth factor B-chain (PDGF-β), the synapsin (Syn), the methyl-CpG binding protein 2 (MeCP2), Ca²⁺/calmodulin-dependent protein kinase II (CaMKII), metabotropic glutamate receptor 2 (mGluR2), NFL, NFH, nβ2, PPE, Enk and EAAT2 promoters. A non-limiting example of a tissue-specific expression elements for astrocytes include the glial fibrillary acidic protein (GFAP) and EAAT2 promoters. A non-limiting example of a tissue-specific expression element for oligodendrocytes includes the myelin basic protein (MBP) promoter.

In one embodiment, the encoded payload construct comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B1-CBX3).

Yu et al. (Molecular Pain 2011, 7:63; the contents of which are herein incorporated by reference in its entirety) evaluated the expression of eGFP under the CAG, EFIα, PGK and UBC promoters in rat DRG cells and primary DRG cells using lentiviral vectors and found that UBC showed weaker expression than the other 3 promoters and there was only 10-12% glia expression seen for all promoters. Soderblom et al. (E. Neuro 2015; the contents of which are herein incorporated by reference in its entirety) evaluated the expression of eGFP in AAV8 with CMV and UBC promoters and AAV2 with the CMV promoter after injection in the motor cortex. Intranasal administration of a plasmid containing a UBC or EFIα promoter showed a sustained airway expression greater than the expression with the CMV promoter (See e.g., Gill et al., Gene Therapy 2001, Vol. 8, 1539-1546; the contents of which are herein incorporated by reference in its entirety). Husain et al. (Gene Therapy 2009; the contents of which are herein incorporated by reference in its entirety) evaluated a HβH construct with a hGUSB promoter, a HSV-1LAT promoter and a NSE promoter and found that the HβH construct showed weaker expression than NSE in mice brain. Passini and Wolfe (J. Virol. 2001, 12382-12392, the contents of which are herein incorporated by reference in its entirety) evaluated the long term effects of the HβH vector following an intraventricular injection in neonatal mice and found that there was sustained expression for at least 1 year. Low expression in all brain regions was found by Xu et al. (Gene Therapy 2001, 8, 1323-1332; the contents of which are herein incorporated by reference in its entirety) when NF-L and NF-H promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920 nucleotide promoter which are both absent in the liver but NFH is abundant in the sensory proprioceptive neurons, brain and spinal cord and NFH is present in the heart. Scn8a is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. Identification of evolutionary conserved, functional noncoding elements in the promoter region of the sodium channel gene SCN8A, Mamm Genome (2007) 18:723-731; and Raymond et al. Expression of Alternatively Spliced Sodium Channel α-subunit genes, Journal of Biological Chemistry (2004) 279(44) 46234-46241; the contents of each of which are herein incorporated by reference in their entireties).

Any of the promoters taught by Yu, Soderblom, Gill, Husain, Passini, Xu, Drews or Raymond may be used in the present inventions.

In one embodiment, the encoded payload construct comprises a promoter which is not cell specific.

In one embodiment, the encoded payload construct comprises an ubiquitin c (UBC) promoter. The UBC promoter may have a size of 300-350 nucleotides. As a non-limiting example, the UBC promoter is 332 nucleotides.

In one embodiment, the encoded payload construct comprises a β-glucuronidase (GUSB) promoter. The GUSB promoter may have a size of 350-400 nucleotides. As a non-limiting example, the GUSB promoter is 378 nucleotides. As a non-limiting example, the construct may be AAV-promoter-CMV/globin intron-hFXN-RBG, where the AAV may be self-complementary and the AAV may be the DJ serotype.

In one embodiment, the payload construct comprises a neurofilament (NFL) promoter. The NFL promoter may have a size of 600-700 nucleotides. As a non-limiting example, the NFL promoter is 650 nucleotides. As a non-limiting example, the construct may be AAV-promoter-CMV/globin intron-hFXN-RBG, where the AAV may be self-complementary and the AAV may be the DJ serotype.

In one embodiment, the payload construct comprises a neurofilament heavy (NFH) promoter. The NFH promoter may have a size of 900-950 nucleotides. As a non-limiting example, the NFH promoter is 920 nucleotides. As a non-limiting example, the construct may be AAV-promoter-CMV/globin intron-hFXN-RBG, where the AAV may be self-complementary and the AAV may be the DJ serotype.

In one embodiment, the payload construct comprises a scn8a promoter. The scn8a promoter may have a size of 450-500 nucleotides. As a non-limiting example, the scn8a promoter is 470 nucleotides. As a non-limiting example, the construct may be AAV-promoter-CMV/globin intron-hFXN-RBG, where the AAV may be self-complementary and the AAV may be the DJ serotype.

In one embodiment, the payload construct comprises a frataxin (FXN) promoter.

In one embodiment, the payload construct comprises a phosphoglycerate kinase 1 (PGK) promoter.

In one embodiment, the payload construct comprises a chicken β-actin (CBA) promoter.

In one embodiment, the payload construct comprises an immediate-early cytomegalovirus (CMV) promoter.

In one embodiment, the payload construct comprises a liver or a skeletal muscle promoter. Non-limiting examples of liver promoters include hAAT and TBG. Non-limiting examples of skeletal muscle promoters include Desmin, MCK and C5-12.

In one embodiment, the payload construct comprises an enhancer element, a promoter and/or a 5′UTR intron. The enhancer element, also referred to herein as an “enhancer,” may be, but is not limited to, a CMV enhancer, the promoter may be, but is not limited to, a CMV, CBA, UBC, GUSB, NSE, Synapsin, MeCP2, and GFAP promoter and the 5′UTR/intron may be, but is not limited to, SV40, and CBA-MVM. As a non-limiting example, the enhancer, promoter and/or intron used in combination may be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter and (9) GFAP promoter.

In one embodiment, the payload construct has an engineered promoter.

In one embodiment, the payload construct comprises a promoter which provides expression for at least 1 month, 2 months, 3 months, 4 months, 5 months, 6 months, 7 months, 8 months, 9 months, 10 months, 11 months, 1 year, 2 years, 3 years 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years.

### Introns

In one embodiment, the payload construct comprises at least one element to enhance the transgene expression such as one or more introns or portions thereof. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In one embodiment, the intron or intron portion may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500.

### Viral Production

The present disclosure provides a method for the generation of parvovirus particle, e.g., AAV particle, by viral genome replication in a viral replication cell comprising contacting the viral replication cell with a payload construct vector and a viral construct vector.

The present disclosure provides a method for producing an AAV particle having enhanced (increased, improved) transduction efficiency comprising the steps of: 1) co-transfecting competent bacterial cells with a bacmid vector and either a viral construct vector and/or payload construct vector, 2) isolating the resultant viral construct vector and payload construct vector and separately transfecting viral replication cells, 3) isolating and purifying resultant payload and viral construct particles comprising viral construct vector or payload construct vector, 4) co-infecting a viral replication cell with both the payload construct vector and viral construct vector, 5) harvesting and purifying the viral particle comprising a parvoviral genome. Production methods are further disclosed in commonly owned and co-pending International Publication No. WO2015191508, the contents of which are herein incorporated by reference in their entirety.

### Particles and Capsids

The invention also provides nucleic acids encoding the mutated or modified virus capsids and capsid proteins of the invention. In some embodiments the capsids are engineered according to the methods of co-owned and co-pending International Publication No. WO2015191508, the contents of which are herein incorporated by reference in their entirety.

Further provided are particles comprising the nucleic acids and cells (in vivo or in culture) comprising the nucleic acids and/or particles of the invention. Suitable particles include without limitation viral particles (e.g., adenovirus, AAV, herpes virus, vaccinia, poxviruses, baculoviruses, and the like), plasmids, phage, YACs, BACs, and the like as are well known in the art. Such nucleic acids, particles and cells can be used, for example, as reagents (e.g., helper packaging constructs or packaging cells) for the production of modified virus capsids or virus particles as described herein.

The molecules of the invention which contain payload constructs include any genetic element (vector) which may be delivered to a host cell, e.g., naked DNA, plasmid, phage, transposon, cosmid, episome, a protein in a non-viral delivery vehicle (e.g., a lipid-based carrier), virus, etc., which transfers the sequences carried thereon. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

The transgene or payload can be carried on any suitable vector, e.g., a plasmid, which is delivered to a host cell. The plasmids useful in this invention may be engineered such that they are suitable for replication and, optionally, integration in prokaryotic cells, mammalian cells, or both. These plasmids contain sequences permitting replication of the transgene in eukaryotes and/or prokaryotes and selection markers for these systems. Selectable markers or reporter genes may include sequences encoding geneticin, hygromicin or purimycin resistance, among others. The plasmids may also contain certain selectable reporters or marker genes that can be used to signal the presence of the vector in bacterial cells, such as ampicillin resistance. Other components of the plasmid may include an origin of replication and an amplicon, such as the amplicon system employing the Epstein Barr virus nuclear antigen. This amplicon system, or other similar amplicon components permit high copy episomal replication in the cells. Preferably, the molecule carrying the transgene or payload is transfected into the cell, where it may exist transiently. Alternatively, the transgene may be stably integrated into the genome of the host cell, either chromosomally or as an episome. In certain embodiments, the transgene may be present in multiple copies, optionally in head-to-head, head-to-tail, or tail-to-tail concatamers. Suitable transfection techniques are known and may readily be utilized to deliver the transgene to the host cell.

In one embodiment, the AAV particles of the invention may be a single-stranded AAV (ssAAV) or a self-complementary AAV (scAAV) described herein or known in the art.

### AAV Capsid Serotypes

In some embodiments, viral vectors and viral particles produced according to the present invention may target to deliver and/or to transfer a payload of interest to specific population of cells in specific anatomical regions (e.g., dopaminergic (DAergic) neurons in the Substantia Nigra (SN)) in the central nervous system).

In some embodiments, viral vectors and viral particles of the present invention may be packaged in a capsid structure or may be capsid free. Such capsid free viral vector donor and/or acceptor sequences such as AAV, are described in, for example, US Publication 20140107186, the content of which is incorporated by reference in its entirety.

In some embodiments, viral vectors and viral particles produced according to the present invention may comprise different capsid proteins, either naturally occurring and/or recombinant, including, but not limited to, AAV1, AAV2, AAV3, AAV4, AAV5, AAV6, AAV7, AAV8, AAV9, AAV10, and AAV11, AAV12, AAVrh8, AAVrh10, AAV-DJ, and AAV-DJ/8 capsid serotypes, or variants thereof (e.g., AAV3A and AAV3B). Nucleic acid sequences encoding one or more AAV capsid proteins useful in the present invention are disclosed in the commonly owned International Publication No. WO2015191508, the contents of which are herein incorporated by reference in their entirety.

In some embodiments, viral vectors and viral particles produced according to the present invention may comprise hybrid serotypes with enhanced transduction to specific cell types of interest in the central nervous system, prolonged transgene expression and/or a safety profile. The hybrid serotypes may be generated by transcapsidation, adsorption of bi-specific antibody to capsid surface, mosaic capsid, and chimeric capsid, and/or other capsid protein modifications.

The naturally occurring AAV Cap gene expresses VP1, VP2, and VP3 capsid proteins are encoded by a single open reading frame of the Cap gene under control of the p40 promoter. In one embodiment, nucleotide sequences encoding VP1, VP2 and VP3 proteins and/or amino acid sequences of AAV VP capsid proteins may be modified for increased efficiency to target to the central nervous system (e.g., CNS tissue tropism). Any of the VP genes of the serotypes selected from, but not limited to, AAV1, AAV2, AAV3, AAV4, AAV5, AAV6, AAV7, AAV8, AAV9, AAV10, and AAV11, AAV12, AAVrh8, AAVrh10, AAV-DJ, and AAV-DJ/8 capsid serotypes, or variants thereof (e.g., AAV3A and AAV3B) may be modified.

In some embodiments, viral vectors and viral particles of the present invention may be further modified toward a specific therapeutic application. The modification may be done by rational mutagenesis of capsid proteins (see, e.g., Pulicherla et al., Mol Ther, 2011, 19: 1070-1078), incorporation of peptide ligands to the capsid, for example a peptide derived from an NMDA receptor agonist for enhanced retrograde transport (Xu et al., Virology, 2005, 341: 203-214), and directed evolution to produce new AAV variants for increased CNS transduction.

In one embodiment, viral vectors and viral particles of the present invention may comprise capsid proteins having sequences of SEQ ID NOs: 1 and 3, which have increased tropism to the brain, of International Publication No. WO2014160092, the content of which is incorporated herein by reference in its entirety.

In one embodiment, viral vectors and viral particles of the present invention may comprise capsid proteins which may target to oligodendrocytes in the central nervous system. The capsid proteins may comprise AAV capsid coding sequence of SEQ ID NO: 1 or AAV capsid proteins comprising amino acid sequences of SEQ ID NOs: 2 to 4 of International Publication No. WO2014052789, the content of which is herein incorporated by reference in its entirety.

In one embodiment, viral vectors and viral particles of the present invention may comprise capsid proteins having increased capacity to cross the blood-brain barrier in CNS as disclosed in U.S. Pat. No. 8,927,514, the content of which is incorporated herein by reference in its entirety. The amino acid sequences and nucleic acid sequences of such capsid proteins may include, but are not limited to, SEQ ID NOs: 2-17 and SEQ ID NOs: 25-33, respectively, of U.S. Pat. No. 8,927,514.

In some embodiments, viral vectors and viral particles of the present invention may comprise AAV2 capsid proteins or variants thereof. Viral particles with AAV2 capsid proteins have been shown to deliver genes to neurons effectively in the brain, retina and spinal cord. In one embodiment, AAV2 capsid proteins may be further modified such as addition of a targeting peptide to the capsid proteins that targets an AAV particle to brain vascular endothelium as disclosed in U.S. Pat. Nos. 6,691,948 and 8,299,215, the contents of each of which are herein incorporated by reference in their entirety. Such viral particles may be used to deliver a functional payload of interest to treat a brain disease such as mucopolysaccharide (MPS).

In some embodiments, viral vectors and viral particles of the present invention may comprise AAV5 capsid proteins or variants thereof. Viral particles with AAV5 capsid proteins can transduce neurons in various regions of the CNS, including the cortex, the hippocampus (HPC), cerebellum, substantia nigra (SN), striatum, globus pallidus, and spinal cord (Burger C et al., Mol Ther., 2004, 10(2): 302-317; Liu G et al., Mol Ther. 2007, 15(2): 242-247; and Colle M et al., Hum, Mol. Genet. 2010, 19(1): 147-158). In one embodiment, viral particles having AA5 capsid proteins with increased transduction to cells in CNS may be those particles from U.S. Pat. No. 7,056,502, the content of which is incorporated herein by reference in its entirety.

In some embodiments, viral vectors and viral particles of the present invention may comprise AAV6 capsid proteins or variants thereof. Recombinant AAV6 serotype can target motor neurons in the spinal cord by Intracerebroventricular (ICV) injection (Dirren E et al., Hum Gene Ther., 2014, 25(2): 109-120). In addition, a study from San Sebastian et al indicated that AAV6 serotype can be retrogradely transported from terminals to neuronal cell bodies in the rat brain (San Sebastian et al, Gen Ther., 2014, 20(12): 1178-1183).

In some embodiments, viral vectors and viral particles of the present invention may comprise AAV8 capsid proteins or variants thereof. Viral particles with AAV8 capsid proteins can transduce neurons, for example in hippocampus (Klein R L et al., Mol Ther., 2006, 13(3): 517-527). In one embodiment, AAV8 capsid proteins may comprise the amino acid sequence of SEQ ID NO: 2 of U.S. Pat. No. 8,318,480, the content of which is herein incorporated by reference in its entirety.

In some embodiments, viral vectors and viral particles of the present invention may comprise AAV9 capsid proteins or variants thereof. AAV9 capsid serotype mediated gene delivery has been observed in the brain with efficient and long-term expression of transgene after intraparenchymal injections to the CNS (Klein R L et al., Eur J Neurosci., 2008, 27: 1615-1625). AAV9 serotype can produce robust and wide-scale neuronal transduction throughout the CNS after a peripheral, systemic (e.g., intravenous) administration in neonatal subjects (Foust K D et al., Nat. Biotechnol., 2009, 27: 59-65; and Duque S et al, Mol Ther., 2009, 17: 1187-1196). Intrathecal (intra-cisterna magna routes) administration of AAV9 serotypes can also produce widespread spinal expression. In one embodiment, AAV9 serotype may comprise an AAV capsid protein having the amino acid sequence of SEQ ID NO: 2 of U.S. Pat. No. 7,198,951, the content of which is incorporated herein by reference in its entirety. In another aspect, AAV 9 serotype may comprise VP1 capsid proteins of SEQ ID NOs: 2, 4 or 6 in which at least one of surface-exposed tyrosine residues in the amino acid sequence is substituted with another amino acid residue, as disclosed in US patent publication No. US20130224836, the content of which is incorporated herein by reference in its entirety.

In some embodiments, viral vectors and viral particles of the present invention may comprise AAVrh10 capsid proteins or variants thereof. Viral particles comprising AAVrh10 capsid proteins can target neurons, other cells as well, in the spinal cord after intrathecal (IT) administration. In one embodiment, AAVrh10 capsid proteins may comprise the amino acid sequence of SEQ ID NO: 81 of EP patent NO: 2341068.

In some embodiments, viral vectors and viral particles of the present invention may comprise AAVDJ capsid proteins, AAVDJ/8 capsid proteins, or variants thereof. Holehonnur et al showed that AVDJ/8 serotype can target neurons within the Basal and Lateral Amygdala (BLA) (Holennur R et al., BMC Neurosci, 2014, February 18:15:28). In one embodiment, AAVDJ capsid proteins and/or AAVDJ/8 capsid proteins may comprise an amino acid sequence comprising a first region that is derived from a first AAV serotype (e.g., AAV2), a second region that is derived from a second AAV serotype (e.g., AAV8), and a third region that is derived from a third AAV serotype (e.g., AAV9), wherein the first, second and third region may include any amino acid sequences that are disclosed in this description.

In some embodiment, viral vectors and viral particles produced according to the present invention may comprise single stranded DNA viral genomes (ssAAVs) or self-complementary AAV genomes (scAAVs). scAAV genomes contain both DNA strands which anneal together to form double stranded DNA. By skipping second strand synthesis, scAAVs allow for rapid expression in the cell.

In one embodiment, the AAV particles of the invention comprise the formula AAV-promoter-transgene. The AAV may be an ssAAV or a scAAV described herein or known in the art.

### Delivery to the CNS

Factors affecting delivery of payloads by parvovirus, e.g., AAV particles to cells of the central nervous system (e.g., parenchyma) as provided by the invention may comprise, but are not limited to, infusion parameters and devices, spatial orientation of the subject, composition physiochemical properties, and viral physiochemical and biochemical properties.

In one embodiment, delivery of payloads by adeno-associated virus (AAV) particles to cells of the central nervous system (e.g., parenchyma) comprises prolonged infusion into cerebrospinal fluid (CSF). CSF is produced by specialized ependymal cells that comprise the choroid plexus located in the ventricles of the brain. CSF produced within the brain then circulates and surrounds the central nervous system including the brain and spinal cord. CSF continually circulates around the central nervous system, including the ventricles of the brain and subarachnoid space that surrounds both the brain and spinal cord, while maintaining a homeostatic balance of production and reabsorption into the vascular system. The entire volume of CSF is replaced approximately four to six times per day or approximately once every four hours, though values for individuals may vary.

In one embodiment, the AAV particles may be delivered by a route to bypass the liver metabolism.

In one embodiment, the AAV particles may be delivered to reduce degradation of the AAV particles and/or degradation of the formulation in the blood.

In one embodiment, the AAV particles may be delivered to bypass anatomical blockages such as, but not limited to the blood brain barrier.

In one embodiment, the AAV particles may be formulated and delivered to a subject by a route which increases the speed of drug effect as compared to oral delivery.

In one embodiment, the AAV particles may be delivery by a method to provide uniform transduction of the spinal cord and dorsal root ganglion (DRG). As a non-limiting example, the AAV particles may be delivered using intrathecal infusion. The intrathecal infusion may be a bolus infusion or it may be a continuous infusion. As another non-limiting example, the AAV particles are delivered using continuous intrathecal infusion over a period of about 10 hours.

In one embodiment, the AAV particles may be delivered to a subject via a single route administration.

In one embodiment, the AAV particles may be delivered to a subject via a multi-site route of administration. For example, a subject may be administered the AAV particles at 2, 3, 4, 5 or more than 5 sites.

In one embodiment, the AAV particles may be formulated. As a non-limiting example the baricity and/or osmolarity of the formulation may be optimized to ensure optimal drug distribution in the central nervous system or a region or component of the central nervous system.

In one embodiment, a subject may be administered the AAV particles described herein using a catheter. The catheter may be placed in the lumbar region or the cervical region of a subject. As a non-limiting example, the catheter may be placed in the lumbar region of the subject. As another non-limiting example, the catheter may be placed in the cervical region of the subject. As yet another non-limiting example, the catheter may be placed in the high cervical region of the subject. As used herein, the “high cervical region” refers to the region of the spinal cord comprising the cervical vertebrae C1, C2, C3 and C4 or any subset thereof.

In one embodiment, a subject may be administered the AAV particles described herein using a bolus infusion. As used herein, a “bolus infusion” means a single and rapid infusion of a substance or composition.

In one embodiment, a subject may be administered the AAV particles described herein using sustained delivery over a period of minutes, hours or days. The infusion rate may be changed depending on the subject, distribution, formulation or another delivery parameter known to those in the art.

In one embodiment, the intracranial pressure may be evaluated prior to administration. The route, volume, AAV particle concentration, infusion duration and/or vector titer may be optimized based on the intracranial pressure of a subject.

In one embodiment, the AAV particles described herein may be delivered by a method which allows even distribution of the AAV particles along the CNS taking into account cerebrospinal fluid (CSF) dynamics. While not wishing to be bound by theory, CSF turnover (TO) occurs approximately 6 times/day or every 4 hours and thus continuous delivery of the AAV particles at a fixed rate, may lead to AAV particles which have distributed throughout the CNS.

In one embodiment, AAV particles are delivered taking into account the oscillating movement of the CSF around the spinal cord. Vortexes are formed by the oscillating movement of the CSF around the cord and these individual vortices combine to form vortex arrays. The arrays combine to form fluid paths for movement of the AAV particles along the spinal cord.

In one embodiment, the delivery method and duration is chosen to provide broad transduction in the spinal cord. As a non-limiting example, intrathecal delivery is used to provide broad transduction along the rostral-caudal length of the spinal cord. As another non-limiting example, multi-site infusions provide a more uniform transduction along the rostral-caudal length of the spinal cord. As yet another non-limiting example, prolonged infusions provide a more uniform transduction along the rostral-caudal length of the spinal cord.

In one embodiment, delivery of AAV particles comprising a viral genome encoding a payload described herein to sensory neurons in the dorsal root ganglion (DRG), ascending spinal cord sensory tracts, and cerebellum will lead to an increased expression of the encoded payload. The increased expression may lead to improved survival and function of various cell types.

In one embodiment, delivery of AAV particles comprising a nucleic acid sequence encoding frataxin to sensory neurons in the dorsal root ganglion (DRG), ascending spinal cord sensory tracts, and cerebellum leads to an increased expression of frataxin. The increased expression of frataxin then leads to improved survival, ataxia (balance) and gait, sensory capability, coordination of movement and strength, functional capacity and quality of life and/or improved function of various cell types.

In one embodiment, the AAV particles may be delivered by systemic delivery. As a non-limiting example, the systemic delivery may be by intravascular administration.

In one embodiment, the AAV particles may be delivered by injection into the CSF pathway. Non-limiting examples of delivery to the CSF pathway include intrathecal and intracerebroventricular administration.

In one embodiment, the AAV particles may be delivered by direct injection into the brain. As a non-limiting example, the brain delivery may be by intrastriatal administration.

### Intracerebroventricular (ICV) Infusion

In one embodiment, delivery of AAV particles to cells of the central nervous system (e.g., parenchyma) is performed by intracerebroventricular (ICV) prolonged infusion. ICV prolonged infusion comprises delivery by injection into the ventricular system of the brain. ICV prolonged infusion may comprise delivery to any of the ventricles of the brain, including, but not limited to, either of the two lateral ventricles left and right, third ventricle, and/or fourth ventricle. ICV prolonged infusion may comprise delivery to any of the foramina, or channels that connect the ventricles, including, but not limited to, interventricular foramina, also called the foramina of Monroe, cerebral aqueduct, and/or central canal. ICV prolonged infusion may comprise delivery to any of the apertures of the ventricular system including, but not limited to, the median aperture (aka foramen of Magendie), right lateral aperture, and/or left lateral aperture (aka foramina of Lushka). In one embodiment, ICV prolonged infusion comprises delivery to the perivascular space in the brain.

### Intrathecal (IT) Infusion

It has been discovered that prolonged continuous intrathecal (IT) infusion leads to stable AAV particle levels within the cerebral spinal fluid (CSF) circulating around the brain and spinal cord by maintaining favorable concentration gradients for AAV particle movement into the parenchyma. Prolonged IT infusion provides increased exposure that favors tissue interactions with AAV by extracellular and intraneuronal processes. In one embodiment extracellular ‘paravascular capture’ comprises the inward movement of AAV along blood vessels. In one embodiment, AAV may move along the outside of neural axons including, but not limited to, nerves such as the dorsal and ventral roots that transect the IT space and are bathed by CSF. Intraneuronal exposure comprises uptake and transport within and along the interior of axons towards (retrograde) or away from (anterograde) the neuronal cell body; AAV has been shown to move in both directions dependent on the serotype.

Stable levels of AAV particles and concentrations gradients induced by prolonged IT infusion increases the total area of spinal cord exposed to efficacious AAV particle concentrations. Consequently, prolonged exposure to the spinal cord will allow for a single site of delivery for widespread neuraxial transfection. As a non-limiting example, the even distribution across targeted neuraxis may avoid hot spots of transduction.

Prolonged IT infusion additionally allows for slower, more controlled infusion that yields more reproducible results. In contrast, bolus delivery can lead to wastage of AAV drug product and ‘hot spots’ comprising uneven, high levels of transduction along the spinal cord or adjacent dorsal root ganglion.

The large size of AAV particles, about 25 nm diameter, leads to steric hindrance wherein there is a limit to the number of AAV particles that may access tissue binding sites and achieve subsequent uptake into cells at any given point in time. Bolus delivery of high numbers of AAV particles over a short period of infusion makes it impossible for much of the delivered AAV dose to reach binding sites for uptake into neurons, astrocytes, oligodendrocytes, microglia and other CNS cells. In contrast, prolonged continuous IT infusion may provide more even and complete distribution of AAV along the neuraxis as AAV concentration reaches equilibrium, thereby reducing the risk of steric hindrance due to the large size of AAV as well as providing a longer timeframe for uptake of AAV into neural cells, tissues, and structures.

This extended timeframe allows for AAV levels in the spinal cord to approach steady state, i.e., the maximum possible level of particles in the CSF for a given infusion rate and concentration. Steady state for AAV levels is reached when the amount of AAV infused into the CSF is equal to AAV clearance rate. The longer that AAV is delivered at or near steady state, the longer there is maintained a favorable diffusion gradient from CSF into parenchyma, which maximizes the opportunity for particles to be transported via extra- and intra-neuronal routes.

While not wishing to be bound by theory, since CSF turnover (TO) occurs approximately 6 times/day or every 4 hours, the steady state described here which is dependent on continuous delivery of AAV at a fixed rate, would predict that AAV particles move and distribute based on CSF flow dynamics, and that overall binding of AAV to tissue is small compared to total AAV particle dose.

The present invention provides administration and/or delivery methods for vectors and viral particles, e.g., AAV particles or AAV, for the treatment or amelioration of diseases or disorders of the CNS. Such methods may involve the inhibition of gene expression, gene replacement or gene activation. Such outcomes are achieved by utilizing the infusion methods taught herein.

In one embodiment, delivery of AAV particles to cells of the central nervous system (e.g., parenchyma) is performed by intrathecal (IT) prolonged infusion. IT prolonged infusion comprises delivery by injection into the subarachnoid space, between the arachnoid membrane and pia mater, which comprises the channels through which CSF circulates. IT prolonged infusion comprises delivery to any area of the subarachnoid space including, but not limited to, perivascular space and the subarachnoid space along the entire length of the spinal cord and surrounding the brain.

### The Spinal Cord

The human spinal cord was first mapped by Bruce, 1901 (Bruce, A., 1901. A Topographical Atlas of the Spinal Cord. Williams and Norgate, London, Available from: www.archive.org/details/cu31924024791406 [24.07.14]) and later by others including Sengul et al., 2013 (Sengul, G., Watson, C., Tanaka, I., Paxinos, G., 2013. Atlas of the Spinal Cord of the Rat, Mouse, Marmoset, Rhesus, and Human. Elsevier Academic Press, San Diego), the contents of each of which is herein incorporated by reference in its entirety. The spinal cord can be divided into 5 regions, into an organization which is derived from the adjacent vertebrae: cervical, thoracic, lumbar, sacral, and coccygeal (caudal) as described in Watson et al., 2015 (Neuroscience Research 93 (2015) 164-175 The spinal cord of the common marmoset (Callithrix jacchus) Charles Watson, Gulgun Senguld, Ikuko Tanakae, Zoltan Rusznakb, and Hironobu Tokunoe), and Pardo et al., 2012 (Toxicologic Pathology, 40: 624-636, 2012 “Technical Guide for Nervous System Sampling of the Cynomolgus Monkey for General Toxicity Studies” Ingrid D. Pardo, Robert H. Garman, Klaus Weber, Walter F. Bobrowski, Jerry F. Hardisty, And Daniel Morton), the contents of each of which is herein incorporated by reference in its entirety. The segments in each region and their numbering are shown in Table 1.

In some instanced, rhesus and cynomolgus monkey each have the same number of segments in each region. Rhesus monkey and Cynomolgus monkeys have 7 or 8 segments in the cervical region. Humans have 7 or 8 segments in the cervical region. Humans, Rhesus monkeys and Cynomolgus monkeys have 12 thoracic segments. Humans have 5 lumbar segments while Rhesus and Cynomolgus monkeys have 7 lumbar segments. The sacral region includes 5 segments in humans, but three segments in Cynomolgus monkey and Rhesus monkey. The coccygeal region has 3 segments in rhesus monkey and cynomolgus monkey, and one segment in human.

TABLE 1
Spinal cord segments in human, cynomolgus and rhesus monkeys

| Spinal Cord | | Cynomolgus | Rhesus |
|---|---|---|---|
| Region | Human | Monkey | Monkey |
| Cervical | C1-C7 | C1-C7 | C1-C7 |
| Thoracic | T1-T12 | T1-T12 | T1-T12 |
| Lumbar | L1-L5 | L1-L7 | L1-L7 |
| Sacral | S1-S5 | S1-S3 | S1-S3 |
| Coccygeal | Co1 | Co1-3 | Co1-3 |
| (caudal) | | | |

Additionally, the spinal cord can also be divided into six regions anatomically and functionally (Sengul et al., 2013 (Sengul, G., Watson, C., Tanaka, I., Paxinos, G., 2013. Atlas of the Spinal Cord of the Rat, Mouse, Marmoset, Rhesus, and Human. Elsevier Academic Press, San Diego), and also Watson et al., Neuroscience Research 93:164-175 (2015)). These regions are the neck muscle region, the upper limb muscle region, the sympathetic outflow region, the lower limb muscle region, the parasympathetic outflow region, and the tail muscle region. These six regions also correlate with territories defined by gene expression during development (see, e.g., Watson et al., supra). The six regions can be defined histologically by the presence or absence of 2 features, the lateral motor column (LMC) and the preganglionic (intermediolateral) column (PGC) (Watson et al., 2015, incorporated herein by reference in its entirety). The limb enlargements are characterized by the presence of a lateral motor column (LMC) and the autonomic regions containing a preganglionic column (PGC). The neck (prebrachial) and tail (caudal) regions have neither an LMC nor a PGC. The limb enlargements and the sympathetic outflow region are marked by particular patterns of hox gene expression in the mouse and chicken, further supporting the division of the spinal cord into these functional regions. Table 2 maps the C, T, L, S and Co designations described in Table 1 to the functional regions according to Sengul et al. and Watson et al. and maps the functional equivalents for Human, Rhesus Monkey, and Japanese Monkey (another macaque). Note: 51 in Rhesus Monkey and L7 in Japanese monkey is located in both crural and postcrural regions.

TABLE 2
Spinal cord regions and sections by function

| Spinal Cord | | Rhesus | Japanese |
|---|---|---|---|
| Region | Human | Monkey | Monkey |
| Neck Muscle | C1-C4 (according | C1-C4 | C1-C3 (as |
| Region | to Bruce) | (according to | described in |
| (prebrachial | C1-C3 (according | Sengul et al.) | Watson et al.) |
| region) | to Sengul et al.) | | |
| Upper limb | C5-T1 (according | C5-T1 | C4-C8 (as |
| Region | to Bruce) | (according to | described in |
| (brachial | C4-T1 (according | Sengul et al.) | Watson et al.) |
| region) | to Sengul et al.) | | |
| Sympathetic | T2-L1 (according | T2-L3 | T1-L2 (as |
| outflow region | to Bruce) | (according to | described in |
| (postbrachial | T2-L1 (according | Sengul et al.) | Watson et al.) |
| region) | to Sengul et al.) | | |
| Lower limb | L2-S2 (according | L4-S1 | L3-L7 (as |
| muscle region | to Bruce) | (according to | described in |
| (crural region) | L3-S2 (according | Sengul et al.) | Watson et al.) |
| | to Sengul et al.) | | |
| Parasympathetic | S3-S4 (according | S1-S3 | L7-S3 (as |
| outflow region | to Bruce) | (according to | described in |
| (postcrural | S3-S5 (according | Sengul et al.) | Watson et al.) |
| region) | to Sengul et al.) | | |
| Tail muscle | S5-Co1 (according | Co1-Co3 | Co1-Co3 (as |
| region | to Bruce) | (according to | described in |
| (caudal region) | Co1 (according | Sengul et al.) | Watson et al.) |
| | to Sengul et al.) | | |

In one embodiment, the catheter for intrathecal delivery may be located in the cervical region. The AAV particles may be delivered in a continuous or bolus infusion.

In one embodiment, the catheter for intrathecal delivery may be located in the lumbar region. The AAV particles may be delivered in a continuous or bolus infusion.

In one embodiment, if continuous delivery of the AAV particles is used, the continuous infusion may be for 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours or more than 24 hours.

In one embodiment, the catheter may be in located at one site in the spine for delivery. As a non-limiting example, the location may be in the cervical or the lumbar region. The AAV particles may be delivered in a continuous or bolus infusion.

In one embodiment, the catheter may be located at more than one site in the spine for multi-site delivery. The AAV particles may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In one embodiment, a subject may be analyzed for spinal anatomy and pathology prior to delivery of the AAV particles described herein. As a non-limiting example, a subject with scoliosis may have a different dosing regimen and/or catheter location compared to a subject without scoliosis.

In one embodiment, the orientation of the spine subject during delivery of the AAV particles may be vertical to the ground.

In another embodiment, the orientation of the spine of the subject during delivery of the AAV particles may be horizontal to the ground.

In one embodiment, the spine of the subject may be at an angle as compared to the ground during the delivery of the AAV particles subject. The angle of the spine of the subject as compared to the ground may be at least 10, 20, 30, 40, 50, 60, 70, 80, 90, 100, 110, 120, 130, 140, 150 or 180 degrees.

In one embodiment, a subject may be delivered the AAV particles herein using two or more delivery routes. As a non-limiting example, the delivery routes may be intrathecal administration and intracerebroventricular administration.

In one embodiment, a subject may be delivered the AAV particles herein at more than one site. As a non-limiting example, the delivery may be a multi-site intrathecal delivery using a bolus injection.

In one embodiment, a subject may be delivered the AAV particles described herein by intrathecal delivery in the lumbar region via a 10 hour bolus injection.

In one embodiment, subjects such as mammals (e.g., non-human primates (NHPs)) are administered by intrathecal (IT) or intracerebroventricular (ICV) infusion the AAV particles described herein. The AAV particles may comprise scAAV or ssAAV, of any of the serotypes described herein, comprising a payload (e.g., a transgene). The dose may be 1×10¹³ to 3×10¹³ vg per subject. The subject may be administered a dose of the AAV particles over an extended period of time such as, but not limited to, 10 ml over 10 hours. The subjects may be evaluated 14-30 days (e.g., 14, 21, 28, or 30 days) after administration to determine the expression of the payload in the subject. Further, the subject may be evaluated prior to administration and after administration to determine changes in behavior and activity such as, but not limited to, tremors, lethargic behavior, motor deficits in limbs, strength, spinal reflex deficits, food consumption. (For AAV9 in Non Human Primates (Cyno) see: Samaranch et al. Human Gene Therapy 23:382-389 April 2012, Samaranch et al. Human Gene Therapy 24: 526-532 May 2013, Samaranch et al. Molecular Therapy 22(2) 329-337 February 2014, Gray et al. Gene Ther. 20(4) 450-459 April 2013; the contents of each of which are herein incorporated by reference in their entireties).

Cross-sections may be labeled according to vertebral segmentation numbering and/or spinal segment numbering. From the mid-thoracic region through the sacral region, the spinal cord is compressed relative to the vertebrae, resulting in a difference of vertebral and spinal levels.

In some embodiments IT prolonged infusion comprises delivery to the cervical, thoracic, and or lumbar regions of the spine. As used herein, IT prolonged infusion into the spine is defined by the vertebral level at the site of prolonged infusion. In some embodiments IT prolonged infusion comprises delivery to the cervical region of the spine at any location including, but not limited to C1, C2, C3, C4, C5, C6, C7, and/or C8. In some embodiments IT prolonged infusion comprises delivery to the thoracic region of the spine at any location including, but not limited to T1, T2, T3, T3, T4, T5, T6, T7, T8, T9, T10, T11, and/or T12. In some embodiments IT prolonged infusion comprises delivery to the lumbar region of the spine at any location including, but not limited to L1, L2, L3, L3, L4, L5, and/or L6. In some embodiments IT prolonged infusion comprises delivery to the sacral region of the spine at any location including, but not limited to S1, S2, S3, S4, or S5. In some embodiments, delivery by IT prolonged infusion comprises one or more than one site of prolonged infusion.

In some embodiments, delivery by IT prolonged infusion may comprise 1, 2, 3, 4, 5, 6, 7, 8, 9, 10, 11, 12, 13, 14, 15, 16, 17, 18, 19, 20, 21, 22, 23, 24, or 25 sites of prolonged infusion. In one embodiment, delivery by IT prolonged infusion comprises at least three sites of prolonged infusion. In one embodiment, delivery by IT prolonged infusion consists of three sites of prolonged infusion. In one embodiment, delivery by IT prolonged infusion comprises three sites of prolonged infusion at C1, T1, and L1.

In one embodiment, intrathecal administration delivers AAV particles to targeted regions of the CNS. Non-limiting examples of regions of the CNS to deliver AAV particles include dorsal root ganglion, dentate nucleus-cerebellum and the auditory pathway.

In one embodiment, intrathecal administration of AAV particles provides peripheral exposure which is as low as possible or a moderate level that is beneficial. As a non-limiting example, intrathecal administration of AAV particles shows almost no peripheral exposure to the liver. As another non-limiting example, intrathecal administration of AAV particles provides an expression level to the heart which treat and/or prevents cardiomyopathies. As yet another non-limiting example, intrathecal administration of AAV particles provides an expression level to the pancreas which treats and/or prevents diabetes.

### Infusion Parameters and Volume

In some embodiments, infusion volume, duration of infusion, infusion patterns and rates for delivery of AAV particles to cells of the central nervous system (e.g., parenchyma) may be determined and regulated. In one embodiment, delivery of AAV particles to cells of the central nervous system (e.g., parenchyma) comprises infusion of up to 1 mL. The infusion may be at least 0.1 mL, 0.2 mL, 0.3 mL, 0.4 mL, 0.5 mL, 0.6 mL, 0.7 mL, 0.8 mL, 0.9 mL, 1 mL or the infusion may be 0.1-0.2 mL, 0.1-0.3 mL, 0.1-0.4 mL, 0.1-0.5 mL, 0.1-0.6 mL, 0.1-0.7 mL, 0.1-0.8 mL, 0.1-0.9 mL, 0.1-1 mL, 0.2-0.3 mL, 0.2-0.4 mL, 0.2-0.5 mL, 0.2-0.6 mL, 0.2-0.7 mL, 0.2-0.8 mL, 0.2-0.9 mL, 0.2-1 mL, 0.3-0.4 mL, 0.3-0.5 mL, 0.3-0.6 mL, 0.3-0.7 mL, 0.3-0.8 mL, 0.3-0.9 mL, 0.3-1 mL, 0.4-0.5 mL, 0.4-0.6 mL, 0.4-0.7 mL, 0.4-0.8 mL, 0.4-0.9 mL, 0.4-1 mL, 0.5-0.6 mL, 0.5-0.7 mL, 0.5-0.8 mL, 0.5-0.9 mL, 0.5-1 mL, 0.6-0.7 mL, 0.6-0.8 mL, 0.6-0.9 mL, 0.6-1 mL, 0.7-0.8 mL, 0.7-0.9 mL, 0.7-1 mL, 0.8-0.9 mL, 0.8-1 mL, or 0.9-1 mL.

In one embodiment, delivery of AAV particles to cells of the central nervous system (e.g., parenchyma) comprises infusion of between about 1 mL to about 120 mL. The infusion may be 1-5 mL, 1-10 mL, 1-15 mL, 1-20 mL, 1-25 mL, 1-30 mL, 1-35 mL, 1-40 mL, 1-45 mL, 1-50 mL, 1-55 mL, 1-60 mL, 1-65 mL, 1-70 mL, 1-75 mL, 1-80 mL, 1-85 mL, 1-90 mL, 1-95 mL, 1-100 mL, 1-105 mL, 1-110 mL, 1-115 mL, 1-120 mL, 5-10 mL, 5-15 mL, 5-20 mL, 5-25 mL, 1-30 mL, 5-35 mL, 5-40 mL, 5-45 mL, 5-50 mL, 5-55 mL, 5-60 mL, 5-65 mL, 5-70 mL, 5-75 mL, 5-80 mL, 5-85 mL, 5-90 mL, 5-95 mL, 5-100 mL, 5-105 mL, 5-110 mL, 5-115 mL, 1-120 mL, 10-15 mL, 10-20 mL, 10-25 mL, 10-30 mL, 10-35 mL, 10-40 mL, 10-45 mL, 10-50 mL, 10-55 mL, 10-60 mL, 10-65 mL, 10-70 mL, 10-75 mL, 10-80 mL, 10-85 mL, 10-90 mL, 10-95 mL, 10-100 mL, 10-105 mL, 10-110 mL, 10-115 mL, 10-120 mL 15-20 mL, 15-25 mL, 15-30 mL, 15-35 mL, 15-40 mL, 15-45 mL, 15-50 mL, 15-55 mL, 15-60 mL, 15-65 mL, 15-70 mL, 15-75 mL, 15-80 mL, 15-85 mL, 15-90 mL, 15-95 mL, 15-100 mL, 15-105 mL, 15-110 mL, 15-115 mL, 15-120 mL, 20-25 mL, 20-30 mL, 20-35 mL, 20-40 mL, 20-45 mL, 20-50 mL, 20-55 mL, 20-60 mL, 20-65 mL, 20-70 mL, 20-75 mL, 20-80 mL, 20-85 mL, 20-90 mL, 20-95 mL, 20-100 mL, 20-105 mL, 20-110 mL, 20-115 mL, 20-120 mL, 25-30 mL, 25-35 mL, 25-40 mL, 25-45 mL, 25-50 mL, 25-55 mL, 25-60 mL, 25-65 mL, 25-70 mL, 25-75 mL, 25-80 mL, 25-85 mL, 25-90 mL, 25-95 mL, 25-100 mL, 25-105 mL, 25-110 mL, 25-115 mL, 25-120 mL, 30-35 mL, 30-40 mL, 30-45 mL, 30-50 mL, 30-55 mL, 30-60 mL, 30-65 mL, 30-70 mL, 30-75 mL, 30-80 mL, 30-85 mL, 30-90 mL, 30-95 mL, 30-100 mL, 30-105 mL, 30-110 mL, 30-115 mL, 30-120 mL, 35-40 mL, 35-45 mL, 35-50 mL, 35-55 mL, 35-60 mL, 35-65 mL, 35-70 mL, 35-75 mL, 35-80 mL, 35-85 mL, 35-90 mL, 35-95 mL, 35-100 mL, 35-105 mL, 35-110 mL, 35-115 mL, 35-120 mL, 40-45 mL, 40-50 mL, 40-55 mL, 40-60 mL, 40-65 mL, 40-70 mL, 40-75 mL, 40-80 mL, 40-85 mL, 40-90 mL, 40-95 mL, 40-100 mL, 40-105 mL, 40-110 mL, 40-115 mL, 40-120 mL, 45-50 mL, 45-55 mL, 45-60 mL, 45-65 mL, 45-70 mL, 45-75 mL, 45-80 mL, 45-85 mL, 45-90 mL, 45-95 mL, 45-100 mL, 45-105 mL, 45-110 mL, 45-115 mL, 45-120 mL, 50-55 mL, 50-60 mL, 50-65 mL, 50-70 mL, 50-75 mL, 50-80 mL, 50-85 mL, 50-90 mL, 50-95 mL, 50-100 mL, 50-105 mL, 50-110 mL, 50-115 mL, 50-120 mL, 55-60 mL, 55-65 mL, 55-70 mL, 55-75 mL, 55-80 mL, 55-85 mL, 55-90 mL, 55-95 mL, 55-100 mL, 55-105 mL, 55-110 mL, 55-115 mL, 55-120 mL, 60-65 mL, 60-70 mL, 60-75 mL, 60-80 mL, 60-85 mL, 60-90 mL, 60-95 mL, 60-100 mL, 60-105 mL, 60-110 mL, 60-115 mL, 60-120 mL, 65-70 mL, 65-75 mL, 65-80 mL, 65-85 mL, 65-90 mL, 65-95 mL, 65-100 mL, 65-105 mL, 65-110 mL, 65-115 mL, 65-120 mL, 70-75 mL, 70-80 mL, 70-85 mL, 70-90 mL, 70-95 mL, 70-100 mL, 70-105 mL, 70-110 mL, 70-115 mL, 70-120 mL, 75-80 mL, 75-85 mL, 75-90 mL, 75-95 mL, 75-100 mL, 75-105 mL, 75-110 mL, 75-115 mL, 75-120 mL, 80-85 mL, 80-90 mL, 80-95 mL, 80-100 mL, 80-105 mL, 80-110 mL, 80-115 mL, 80-120 mL, 85-90 mL, 85-95 mL, 85-100 mL, 85-105 mL, 85-110 mL, 85-115 mL, 85-120 mL, 90-95 mL, 90-100 mL, 90-105 mL, 90-110 mL, 90-115 mL, 90-120 mL, 95-100 mL, 95-105 mL, 95-110 mL, 95-115 mL, 95-120 mL, 100-105 mL, 100-110 mL, 100-115 mL, 100-120 mL, 105-110 mL, 105-115 mL, 105-120 mL, 110-115 mL, 110-120 mL, or 115-120 mL.

In one embodiment, delivery of AAV particles to cells of the central nervous system (e.g., parenchyma) may comprise an infusion of about 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 mL. In one embodiment, delivery of AAV particles to cells of the central nervous system (e.g., parenchyma) comprises of infusion of 1 mL.

In one embodiment, delivery of AAV particles to cells of the central nervous system (e.g., parenchyma) comprises of infusion of at least 1 mL. In one embodiment, delivery of AAV particles to cells of the central nervous system (e.g., parenchyma) comprises infusion of at least 3 mL. In one embodiment, delivery of AAV particles to cells of the central nervous system (e.g., parenchyma) comprises of infusion of 3 mL. In one embodiment, delivery of AAV particles to cells of the central nervous system (e.g., parenchyma) comprises infusion of at least 10 mL. In one embodiment, delivery of AAV particles to cells of the central nervous system (e.g., parenchyma) consists of infusion of 10 mL.

In one embodiment, the serotype of the AAV particles described herein may depend on the desired distribution, transduction efficiency and cellular targeting required. As described by Sorrentino et al. (comprehensive map of CNS transduction by eight adeno-associated virus serotypes upon cerebrospinal fluid administration in pigs, Molecular Therapy accepted article preview online 7 Dec. 2015; doi:10.1038/mt.2015.212; the contents of which are herein incorporated by reference in its entirety), AAV serotypes provided different distributions, transduction efficiencies and cellular targeting. In order to provide the desired efficacy, the AAV serotype needs to be selected that best matches not only the cells to be targeted but also the desired transduction efficiency and distribution.

### Duration of Infusion: Bolus Infusion

In one embodiment, delivery of AAV particles to cells of the central nervous system (e.g., parenchyma) comprises infusion by bolus injection with a duration of less than 30 minutes. In one embodiment, infusion by bolus injection comprises injection with a duration of less than 20 minutes. In one embodiment, infusion by bolus injection comprises injection with a duration of less than 10 minutes. In one embodiment, infusion by bolus injection comprises injection with a duration of less than 10 seconds. In one embodiment, infusion by bolus injection comprises injection with a duration of between 10 seconds to 10 minutes. In one embodiment, infusion by bolus injection comprises injection with a duration of 10 minutes. In one embodiment, infusion by bolus injection consists of injection with a duration of 10 minutes.

In one embodiment, delivery of AAV particles to cells of the central nervous system (e.g., parenchyma) comprises infusion by at least one bolus injection. In one embodiment, delivery may comprise infusion by 1, 2, 3, 4, 5, 6, 7, 8, 9, or 10 bolus injections. In one embodiment, delivery may comprise infusion by at least three bolus injections. In one embodiment, delivery comprises infusion by three bolus injections. In one embodiment, delivery of AAV to cells of the central nervous system (e.g., parenchyma) consists of infusion by three bolus injections.

In one embodiment, delivery of AAV particles to cells of the central nervous system (e.g., parenchyma) comprising infusion of more than one bolus injection further comprises an interval of at least one hour between injections. In one embodiment, delivery of AAV particles to cells of the central nervous system (e.g., parenchyma) comprising infusion of more than one bolus injection may further comprise an interval of 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72, 78, 84, 90, 96, 108, or 120 hour(s) between injections. In one embodiment, delivery comprising infusion of more than one bolus injection further comprises an interval of one hour between injections. In one embodiment, delivery consists of infusion by three bolus injections at an interval of one hour.

In one embodiment, DRG and/or cortical brain expression may be higher with shorter, high concentration infusions.

### Prolonged Infusion

In one embodiment, delivery of AAV particles to cells of the central nervous system (e.g., parenchyma) comprises prolonged infusion of pharmaceutically acceptable composition comprising AAV particles over a duration of at least 10 minutes. In one embodiment, delivery comprises prolonged infusion over a duration of between 30 minutes and 60 minutes. In one embodiment, delivery may comprise prolonged infusion of over a duration of 0.17, 0.33, 0.5, 0.67, 0.83, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 hour(s). In one embodiment, delivery comprises prolonged infusion over a duration of one hour. In one embodiment, delivery of AAV particles to cells of the central nervous system (e.g., parenchyma) consists of prolonged infusion over a duration of one hour.

In one embodiment, delivery of AAV particles to cells of the central nervous system (e.g., parenchyma) comprises prolonged infusion over a duration of 10 hours. In one embodiment, delivery of AAV particles to cells of the central nervous system (e.g., parenchyma) consists of prolonged infusion over a duration of 10 hours. In one embodiment, prolonged infusion may yield more homogenous levels of protein expression across the spinal cord, as compared to bolus dosing at one or multiple sites. In one embodiment, dentate nucleus expression may increase with prolonged infusions.

### Single and Multiple Rounds of Dosing

In one embodiment, delivery of AAV particles to cells of the central nervous system (e.g., parenchyma) comprises prolonged infusion of at least one dose. In one embodiment, delivery comprises prolonged infusion of one dose. In one embodiment, delivery of AAV to cells of the central nervous system (e.g., parenchyma) may comprise prolonged infusion of 1, 2, 3, 4, 5, 6, 7, 8, 9, or 10 dose(s).

### Interval of Dosing

In one embodiment, delivery of AAV particles to cells of the central nervous system (e.g., parenchyma) comprising prolonged infusion of more than one dose further comprises an interval of at least one hour between doses. In one embodiment, delivery may comprise an interval of 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72, 78, 84, 90, 96, 108, or 120 hour(s) between doses. In one embodiment, delivery comprises an interval of 24 hours between doses. In one embodiment, delivery consists of three prolonged infusion doses at an interval of 24 hours.

### Infusion Patterns: Simple (Constant)

In one embodiment, delivery of AAV particles to cells of the central nervous system (e.g., parenchyma) may comprise a constant rate of prolonged infusion. As used herein, a “constant rate” is a rate that stays about the same during the prolonged infusion.

### Infusion Patterns: Ramped

In one embodiment, delivery of AAV particles to cells of the central nervous system (e.g., parenchyma) may comprise a ramped rate of prolonged infusion where the rate either increases or decreases over time. As a non-limiting example, the rate of prolonged infusion increases over time. As another non-limiting example, the rate of prolonged infusion decreases over time.

### Infusion Patterns: Complex

In one embodiment, delivery of AAV particles to cells of the central nervous system (e.g., parenchyma) may comprise a complex rate of prolonged infusion wherein the rate of prolonged infusion alternates between high and low rates of prolonged infusion over time.

### Prolonged Infusion Rate

In one embodiment, delivery of AAV particles to cells of the central nervous system (e.g., parenchyma) comprises a rate of delivery may be defined by [VG/hour=mL/hour*VG/mL] wherein VG is viral genomes, VG/mL is composition concentration, and mL/hour is rate of prolonged infusion. In accordance with the present invention, the

In one embodiment, delivery of AAV to cells of the central nervous system (e.g., parenchyma) may comprise a rate of prolonged infusion between about 0.1 mL/hour and about 25.0 mL/hour (or higher if CSF pressure does not increase to dangerous levels). In some embodiments, delivery may comprise a rate of prolonged infusion of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, or 25.0 mL/hour. In some embodiments, delivery may comprise a rate of prolonged infusion of about 10, 20 30, 40, or 50 mL/hr. In one embodiment, delivery of AAV particles to cells of the central nervous system (e.g., parenchyma) comprises a rate of prolonged infusion of 1.0 mL/hour. In one embodiment, delivery consists of a rate of prolonged infusion of 1.0 mL/hour. In one embodiment, delivery of AAV to cells of the central nervous system (e.g., parenchyma) comprises a rate of prolonged infusion of 1.5 mL/hour. In one embodiment, delivery of AAV particles to cells of the central nervous system (e.g., parenchyma) consists of a rate of prolonged infusion of 1.5 mL/hour.

### Prolonged Infusion Dosing: Total Dose

In one embodiment, delivery of AAV particles to cells of the central nervous system (e.g., parenchyma) comprises prolonged infusion of at least one dose, or two or more doses. The interval between doses may be at least one hour, or between 1 hour and 120 hours. In one embodiment, the total dose of viral genomes delivered to cells of the central nervous system (e.g., parenchyma) defined by the equation [Total Dose VG=VG/mL*mL*# of doses] wherein VG is viral genomes and VG/mL is viral genome concentration. In accordance with the present invention, the total dose may be between about 1×10⁶ VG and about 1×10¹⁶ VG.

### Infusion Compositions

In some embodiments, a composition comprising AAV particles delivered to cells of the central nervous system (e.g., parenchyma) may have a certain range of concentrations, pH, baricity (i.e. density of solution), osmolarity, temperature, and other physiochemical and biochemical properties that benefit the delivery of AAV particles to cells of the central nervous system (e.g., parenchyma).

In one embodiment, delivery of AAV particles to cells of the central nervous system (e.g., parenchyma) may comprise a total dose between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×106, 1×10⁷, 2×10⁷, 3×10⁷, 4×10⁷, 5×10⁷, 6×10⁷, 7×10⁷ 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 1.9×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG. As a non-limiting example, the total dose is 1×10¹³ VG. As another non-limiting example, the total dose is 3×10¹³ VG. As another non-limiting example, the total dose is 3.73×10¹⁰ VG. As another non-limiting example, the total dose is 1.9×10¹⁰ VG. As another non-limiting example, the total dose is 2.5×10¹¹ VG. As another non-limiting example, the total dose is 5×10¹¹ VG. As another non-limiting example, the total dose is 1×10¹² VG. As another non-limiting example, the total dose is 5×10¹² VG.

In one embodiment, delivery of AAV particles to cells of the central nervous system (e.g., parenchyma) comprises a total dose of 5×10¹⁰ VG. In one embodiment, delivery consists of a total dose of 5×10¹⁰ VG. In one embodiment, delivery comprises a total dose of 3×10¹³ VG. In one embodiment, delivery of AAV to cells of the central nervous system (e.g., parenchyma) consists of a total dose of 3×10¹³ VG.

### Pressure

In one embodiment, delivery of AAV particles to cells of the central nervous system (e.g., parenchyma) may comprise a rate of prolonged infusion wherein the rate of prolonged infusion exceeds the rate of CSF absorption. In some embodiments, CSF pressure may increase wherein the rate of delivery is greater than the rate of clearance. In one embodiment, increased CSF pressure may increase delivery of AAV particles to cells of the central nervous system (e.g., parenchyma of brain and spinal cord). In one embodiment, delivery of AAV to cells of the central nervous system (e.g., parenchyma) may comprise an increase in sustained CSF pressure between about 1% and about 25%. In some embodiments, delivery may comprise an increase in sustained CSF pressure of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%.

Although the descriptions of pharmaceutical compositions, e.g., AAV comprising a payload to be delivered, provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, human patients or subjects. For the purposes of the present disclosure, the phrase “active ingredient” generally refers either to the viral particle carrying the payload or to the payload delivered by the viral particle as described herein.

Formulations of the AAV pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered.

### Prolonged Infusion Composition Concentration

In one embodiment, delivery of AAV particles to cells of the central nervous system (e.g., parenchyma) may comprise a composition concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10°, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL. In one embodiment, delivery comprises a composition concentration of 1×10¹³ VG/mL. In one embodiment, delivery consists of a composition concentration of 1×10¹³ VG/mL. In one embodiment, delivery comprises a composition concentration of 3×10¹² VG/mL. In one embodiment, delivery consists of a composition concentration of 3×10¹² VG/mL.

### Composition pH

In one embodiment, delivery of AAV to cells of the central nervous system (e.g., parenchyma) comprises a buffered composition of between pH 4.5 and 8.0. In some embodiments, delivery may comprise a buffered composition of about pH 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In one embodiment, delivery comprises a buffered composition of pH 7.4, which is considered physiological pH. In one embodiment, delivery comprises a buffered composition of pH 7.0. In one embodiment, buffer strength, or ability to hold pH, is relatively very low allowing the infused composition to quickly adjust to the prevailing physiological pH of the CSF (˜pH7.4).

### Composition Baricity

It is known in the art that CSF comprises a baricity, or density of solution, of approximately 1 g/mL at 37° C. In one embodiment, delivery of AAV particles to cells of the central nervous system (e.g., parenchyma) comprises an isobaric composition wherein the baricity of the composition at 37° C. is approximately 1 g/mL. In one embodiment, delivery comprises a hypobaric composition wherein the baricity of the composition at 37° C. is less than 1 g/mL. In one embodiment, delivery comprises a hyperbaric composition wherein the baricity of the composition at 37° C. is greater than 1 g/mL. In one embodiment, delivery comprises a hyperbaric composition wherein the baricity of the composition at 37° C. is increased by addition of approximately 5% to 8% dextrose. In one embodiment, delivery comprises a hyperbaric composition wherein the baricity of the composition at 37° C. is increased by addition of 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, or 8.0% dextrose.

### Composition Osmolarity

In one embodiment, delivery of AAV particles to cells of the central nervous system (e.g., parenchyma) comprises co-administration of intravenous agents that increase serum osmolarity. As used herein, “co-administered” means the administration of two or more components. Co-administration refers to the administration of two or more components simultaneously or with a time lapse between administration such as 1 second, 5 seconds, 10 seconds, 15 seconds, 30 seconds, 45 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 1 hour, 1.5 hours, 2 hours, 2.5 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 1.5 days, 2 days, or more than 3 days.

In one embodiment, delivery comprises co-administration of intravenous mannitol. In one embodiment, delivery comprises co-administration of approximately 0.25 to 1.0 g/kg intravenous mannitol. In one embodiment, delivery comprises co-administration of 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00 g/kg intravenous mannitol.

### Composition Temperature

In one embodiment, delivery of AAV particles to cells of the central nervous system (e.g., parenchyma) comprises a composition wherein the temperature of the composition is 37° C. In one embodiment, delivery comprises a composition wherein the temperature of the composition is between approximately 20° C. and 26° C. In one embodiment, delivery comprises a composition wherein the temperature of the composition is approximately 20.0° C., 20.1° C., 20.2° C., 20.3° C., 20.4° C., 20.5° C., 20.6° C., 20.7° C., 20.8° C., 20.9° C., 21.0° C., 21.1° C., 21.2° C., 21.3° C., 21.4° C., 21.5° C., 21.6° C., 21.7° C., 21.8° C., 21.9° C., 22.0° C., 22.1° C., 22.2° C., 22.3° C., 22.4° C., 22.5° C., 22.6° C., 22.7° C., 22.8° C., 22.9° C., 23.0° C., 23.1° C., 23.2° C., 23.3° C., 23.4° C., 23.5° C., 23.6° C., 23.7° C., 23.8° C., 23.9° C., 24.0° C., 24.1° C., 24.2° C., 24.3° C., 24.4° C., 24.5° C., 24.6° C., 24.7° C., 24.8° C., 24.9° C., 25.0° C., 25.1° C., 25.2° C., 25.3° C., 25.4° C., 25.5° C., 25.6° C., 25.7° C., 25.8° C., 25.9° C., or 26.0° C.

### Drug Physiochemical & Biochemical Properties

In one embodiment, delivery of parvovirus e.g., AAV particles to cells of the central nervous system (e.g., parenchyma) comprises a composition wherein the AAV capsid is hydrophilic. In one embodiment, delivery comprises a composition wherein the AAV capsid is lipophilic.

In one embodiment, delivery of AAV particles to cells of the central nervous system (e.g., parenchyma) comprises a composition wherein the AAV capsid targets a specific receptor. In one embodiment, delivery of AAV particles to cells of the central nervous system (e.g., parenchyma) comprises a composition wherein the AAV capsid further comprises a specific ligand.

In some embodiments, delivery of AAV particles to cells of the central nervous system (e.g., parenchyma) comprises a composition wherein the AAV capsid may be, but is not limited to, AAV1, AAV2, AAV3, AAV4, AAV5, AAV6, AAV7, AAV8, AAV9, AAV10, AAV11, AAV12, AAVrh8, AAVrh10, AAV-DJ, and/or AAV-DJ8.

In one embodiment, delivery of AAV particles to cells of the central nervous system (e.g., parenchyma) comprises a composition wherein the AAV further comprises a self-complementary (SC) genome. In one embodiment, delivery comprises a composition wherein the AAV further comprises a single stranded (SS) genome.

In one embodiment, a self-complementary (sc) vector may be used to yield higher expression than the corresponding single stranded vector.

In one embodiment, delivery of AAV particles to cells of the central nervous system (e.g., parenchyma) comprises a composition wherein the AAV genome further comprises a cell specific promoter region. In one embodiment, delivery comprises a composition wherein the AAV genome further comprises a ubiquitous promoter region.

### Spatial Orientation: Body Angle

In one embodiment, delivery of AAV particles to cells of the central nervous system (e.g., parenchyma) comprises administration to a horizontal subject. In one embodiment, delivery comprises administration to a vertical subject. In one embodiment, delivery comprises administration to a subject at an angle between approximately horizontal 0° to about vertical 90°. In one embodiment, delivery comprises administration to a subject at an angle of 0°, 1°, 2°, 3°, 4°, 5°, 6°, 7°, 8°, 9°, 10°, 11°, 12°, 13°, 14°, 15°, 16°, 17°, 18°, 19°, 20°, 21°, 22°, 23°, 24°, 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, 65°, 66°, 67°, 68°, 69°, 70°, 71°, 72°, 73°, 74°, 75°, 76°, 77°, 78°, 79°, 80°, 81°, 82°, 83°, 84°, 85°, 86°, 87°, 88°, 89°, 90°.

### Spatial Orientation: Change in the Orientation/Slope of Subject Body Position Over Time

In one embodiment, delivery of AAV particles to cells of the central nervous system (e.g., parenchyma) comprises administration to a subject wherein the angle of the subject changes over time from horizontal to vertical head up or vertical head down. In one embodiment, delivery comprises administration to a subject wherein the angle of the subject changes over time from vertical to horizontal.

In one embodiment, delivery comprises administration to a subject wherein the angle of the subject changes over time in two planes from vertical to horizontal as well as rotation around the long axis of the body. In combination, any % angle of the body can be realized between horizontal to vertical and rotationally left or right.

### Delivery Devices

In some embodiments, delivery of AAV particles to cells of the central nervous system (e.g., parenchyma) comprises a prolonged infusion pump or device. In some embodiments, the device may be a pump or comprise a catheter for administration of compositions of the invention across the blood brain barrier. Such devices include but are not limited to a pressurized olfactory delivery device, iontophoresis devices, multi-layered microfluidic devices, and the like. Such devices may be portable or stationary. They may be implantable or externally tethered to the body or combinations thereof.

Devices for administration may be employed for delivery of AAV particles to cells of the central nervous system (e.g., parenchyma) according to the present invention according to single, multi- or split-dosing regimens taught herein.

Method and devices known in the art for multi-administration to cells, organs and tissues are contemplated for use in conjunction with the methods and compositions disclosed herein as embodiments of the present invention. These include, for example, those methods and devices having multiple needles, hybrid devices employing for example lumens or catheters as well as devices utilizing heat, electric current or radiation driven mechanisms.

In one embodiment, the AAV particles may be delivered using an infusion port described herein and/or one that is known in the art.

In one embodiment, the AAV particles may be delivered using an infusion pump and/or an infusion port. The infusion pump and/or the infusion port may be one described herein or one known in the art such as, but not limited to, SYNCHROMED® II by Medtronic. The infusion pump may be programmed at a fixed rate or a variable rate for controlled delivery. The stability of the AAV particles and formulations thereof as well as the leachable materials should be evaluated prior to use.

In one embodiment, the devices described herein to deliver to a subject the above-described AAV particles may also include a tip protection device (e.g., for catheters and/or stereotactic fixtures of microcatheters). Non-limiting examples of protection devices are described in US Patent Publication No. US20140371711 and International Patent Publication No. WO2014204954, the contents of each of which are herein incorporated by reference in their entireties. The tip protection device may include an elongate body having a central lumen extending longitudinally therethrough, the lumen being sized and configured to slidably receive a catheter, and a locking mechanism configured to selectively maintain the elongate body in a fixed longitudinal position relative to a catheter inserted through the central lumen.

In one embodiment, the AAV particles may be delivered to a subject using a convection-enhanced delivery device. Non-limiting examples of targeted delivery of drugs using convection are described in US Patent Publication Nos. US20100217228, US20130035574 and US20130035660 and International Patent Publication No. WO2013019830 and WO2008144585, the contents of each of which are herein incorporated by reference in their entireties. The convection-enhanced delivery device may be a microfluidic catheter device that may be suitable for targeted delivery of drugs via convection, including devices capable of multi-directional drug delivery, devices that control fluid pressure and velocity using the venturi effect, and devices that include conformable balloons. As a non-limiting example, the convention-enhanced delivery device uses the venturi effect for targeted delivery of drugs as described in US Patent Publication No. US20130035574, the contents of which are herein incorporation by reference in its entirety. As another non-limiting example, the convention-enhanced delivery device uses the conformable balloons for targeted delivery of drugs as described in US Patent Publication No. US20130035660, the contents of which are herein incorporation by reference in its entirety. As another non-limiting example, the convection enhanced delivery device may be a CED catheter from Medgenesis Therapeutix such as those described in International Patent Publication No. WO2008144585 and US Patent No. US20100217228, the contents of each of which are herein incorporated by reference in their entireties. As another non-limiting example, the AAV particles may be in a liposomal composition for convection enhanced delivery such as the liposomal compositions from Medgenesis Therapeutix described in International Patent Publication No. WO2010057317 and US Patent No. US20110274625, the contents of each of which are herein incorporated by reference in their entireties, which may comprise a molar ratio of DSPC:DSPG:CHOL of 7:2:1.

In one embodiment, the catheter may be a neuromodulation catheter. Non-limiting examples of neuromodulation catheters include those taught in US Patent Application No. US20150209104 and International Publication Nos. WO2015143372, WO2015113027, WO2014189794 and WO2014150989, the contents of each of which are herein incorporated by reference in their entireties.

In one embodiment, the AAV particles may be delivered using an injection device which has a basic form of a stiff tube with holes of a selectable size at selectable places along the tube. This is a device which may be customized depending on the subject or the fluid being delivered. As a non-limiting example, the injection device which comprises a stiff tube with holes of a selectable size and location may be any of the devices described in U.S. Pat. Nos. 6,464,662, 6,572,579 and International Patent Publication No. WO2002007809, the contents of each of which are herein incorporated by reference in their entireties.

In one embodiment, the AAV particles may be delivered to a subject who is using or who has used a treatment stimulator for brain diseases. Non-limiting examples include treatment stimulators from THERATAXIS™ and the treatment stimulators described in International Patent Publication No. WO2008144232, the contents of which are herein incorporated by reference in its entirety.

In one embodiment, the AAV particles may be delivered to a defined area using a medical device which comprises a sealing system proximal to the delivery end of the device. Non-limiting examples of medical device with can deliver AAV particles to a defined area includes U.S. Pat. No. 7,998,128, US Patent Application No. US20100030102 and International Patent Publication No. WO2007133776, the contents of each of which are herein incorporated by reference in their entireties.

In one embodiment, the AAV particle may be delivered over an extended period of time using an extended delivery device. Non-limiting examples of extended delivery devices are described in International Patent Publication Nos. WO2015017609 and WO2014100157, U.S. Pat. No. 8,992,458, and US Patent Publication Nos. US20150038949, US20150133887 and US20140171902, the contents of each of which are herein incorporated by reference in their entireties. As a non-limiting example, the devices used to deliver the AAV particles are CED devices with various features for reducing or preventing backflow as in International Patent Publication No. WO2015017609 and US Patent Publication No. US20150038949, the contents of each of which are herein incorporated by reference in their entireties. As another non-limiting example, the devices used to deliver the AAV particles are CED devices which include a bullet-shaped nose proximal to a distal fluid outlet where the bullet-shaped nose forms a good seal with surrounding tissue and helps reduce or prevent backflow of infused fluid as described in U.S. Pat. No. 8,992,458, US Patent Publication Nos. US20150133887 and US20140171902 and International Patent Publication No. WO2014100157, the contents of each of which are herein incorporated by reference by their entireties. As another non-limiting example, the catheter may be made using micro-electro-mechanical systems (MEMS) technology to reduce backflow as described by Brady et al. (Journal of Neuroscience Methods 229 (2014) 76-83), the contents of which are herein incorporated by reference in its entirety.

In one embodiment, the AAV particles may be delivered using an implantable delivery device. Non-limiting examples of implantable devices are described by and sold by Codman Neuro Sciences (Le Lode, CH). The implantable device may be an implantable pump such as, but not limited to, those described in U.S. Pat. Nos. 8,747,391, 7,931,642, 7,637,897, and 6,755,814 and US Patent Publication No. US20100069891, the contents of each of which are herein incorporated by reference in their entireties. The implantable device (e.g., a fluidic system) may have the flow rate accuracy of the device optimized by the methods described in U.S. Pat. Nos. 8,740,182 and 8,240,635, and US Patent Publication No. US20120283703, the contents of each of which are herein incorporated by reference in its entirety. As a non-limiting example, the duty cycle of the valve of a system may be optimized to achieve the desired flow rate. The implantable device may have an electrokinetic actuator for adjusting, controlling or programming fine titration of fluid flow through a valve mechanism without intermixing between the electrolyte and fluid. As a non-limiting example, the electrokinetic actuator may be any of those described in U.S. Pat. No. 8,231,563 and US Patent Publication No. US20120283703, the contents of which are herein incorporated by reference in its entirety. Fluids of an implantable infusion pump may be monitored using methods known in the art and those taught in U.S. Pat. No. 7,725,272, the contents of which are herein incorporated by reference in its entirety.

In one embodiment, the delivery of the AAV particles in a subject may be determined and/or predicted using the prediction methods described in International Patent Publication No. WO2001085230, the contents of which are herein incorporated by reference in its entirety.

In one embodiment, a subject may be imaged prior to, during and/or after administration of the AAV particles. The imaging method may be a method known in the art and/or described herein. As a non-limiting example, the imaging method which may be used to classify brain tissue includes the medical image processing method described in U.S. Pat. Nos. 7,848,543, 9,101,282 and EP Application No. EP1768041, the contents of each of which are herein incorporated by reference in their entireties. As yet another non-limiting example, the physiological states and the effects of treatment of a neurological disease in a subject may be tracked using the methods described in US Patent Publication No. US20090024181, the contents of which are herein incorporated by reference in its entirety.

In one embodiment, a device may be used to deliver the AAV particles where the device creates one or more channels, tunnels or grooves in tissue in order to increase hydraulic conductivity. These channels, tunnels or grooves will allow the AAV particles to flow and produce a predictable infusion pattern. Non-limiting examples of this device is described in U.S. Pat. No. 8,083,720, US Patent Application No. US20110106009, and International Publication No. WO2009151521, the contents of each of which are herein incorporated by reference in its entirety.

In one embodiment, the flow of a composition comprising the AAV particles may be controlled using acoustic waveform outside the target area. Non-limiting examples of devices, methods and controls for using sonic guidance to control molecules is described in US Patent Application No. US20120215157, U.S. Pat. No. 8,545,405, International Patent Publication Nos. WO2010096495 and WO2010080701, the contents of each of which are herein incorporated by reference in their entireties.

In one embodiment, the flow of a composition comprising the AAV particles may be modeled prior to administration using the methods and apparatus described in U.S. Pat. Nos. 6,549,803 and 8,406,850 and US Patent Application No. US20080292160, the content of each of which is incorporated by reference in their entireties. As a non-limiting example, the physiological parameters defining edema induced upon infusion of fluid from an intraparenchymally placed catheter may be estimated using the methods described in U.S. Pat. No. 8,406,850 and US Patent Application No. US20080292160, the contents of which is herein incorporated by reference in its entirety.

In one embodiment, a surgical alignment device may be used to deliver the AAV particles to a subject. The surgical alignment device may be a device described herein and/or is known in the art. As a non-limiting example, the surgical alignment device may be controlled remotely (i.e., robotic) such as the alignment devices described in U.S. Pat. Nos. 7,366,561 and 8,083,753, the contents of each of which is incorporated by reference in their entireties.

In one embodiment, an intraparenchymal (IPA) catheter from Alcyone may be used to deliver the AAV particles described herein.

In another embodiment, an intraparenchymal catheter from Atanse may be used to deliver the AAV particles described herein.

In one embodiment, the distribution of the AAV particles described herein may be evaluated using imaging technology from Therataxis and/or Brain Lab.

### Treatment and Pharmaceutical Compositions

### Formulation

The AAV particles of the invention can be formulated using one or more excipients to: (1) increase stability; (2) increase cell transfection or transduction; (3) permit the sustained or delayed release; (4) alter the biodistribution (e.g., target the viral particle to specific tissues or cell types); (5) increase the translation of encoded protein in vivo; (6) alter the release profile of encoded protein in vivo and/or (7) allow for regulatable expression of the payload.

Formulations of the present invention can include, without limitation, saline, liposomes, lipid nanoparticles, polymers, peptides, proteins, cells transfected with viral vectors (e.g., for transplantation into a subject) and combinations thereof.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

In one embodiment, the AAV particles of the invention may be formulated in PBS with 0.001% of pluronic acid (F-68) at a pH of about 7.0.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient (e.g. AAV particle), the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the AAV particle formulations described herein may contain a nucleic acid encoding at least one payload. As a non-limiting example, the formulations may contain a nucleic acid encoding 1, 2, 3, 4 or 5 payloads. In one embodiment the formulation may contain a nucleic acid encoding a payload construct encoding proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic and cytoskeletal proteins, intracellular membrane bound proteins, nuclear proteins, proteins associated with human disease and/or proteins associated with non-human diseases. In one embodiment, the formulation contains at least three payload constructs encoding proteins.

The formulations of the invention can include one or more excipients, each in an amount that together increases the stability of the AAV particle, increases cell transfection or transduction by the viral particle, increases the expression of viral particle encoded protein, and/or alters the release profile of AAV particle encoded proteins. In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

### Inactive Ingredients

In some embodiments, AAV formulations may comprise at least one excipient which is an inactive ingredient. As used herein, the term “inactive ingredient” refers to one or more agents that do not contribute to the activity of the pharmaceutical composition included in formulations. In some embodiments, all, none or some of the inactive ingredients which may be used in the formulations of the present invention may be approved by the US Food and Drug Administration (FDA).

Formulations of AAV particles disclosed herein may include cations or anions. In one embodiment, the formulations include metal cations such as, but not limited to, Zn2+, Ca2+, Cu2+, Mg+ and combinations thereof.

### Administration

The AAV particles of the present invention may be administered by any route which results in a therapeutically effective outcome. These include, but are not limited to epidural, peridural, subdural (in particular delivery of AAV over one or more targeted regions of the neocortex), intracerebral (into the cerebrum), intracerebroventricular (into the cerebral ventricles), intrathecal (into the spinal canal or within the cerebrospinal fluid at any level of the cerebrospinal axis), intradiscal (within a disc), intradural (within or beneath the dura), intraspinal (within the vertebral column), caudal block, diagnostic, nerve block, or spinal. In specific embodiments, compositions may be administered in a way which allows them cross the blood-brain barrier, vascular barrier, or other epithelial barrier. In one embodiment, a formulation for a route of administration may include at least one inactive ingredient.

In one embodiment, the AAV particles may be delivered by systemic delivery. As a non-limiting example, the systemic delivery may be by intravascular administration.

In one embodiment, the AAV particles may be delivered by injection into the CSF pathway. Non-limiting examples of delivery to the CSF pathway include intrathecal and intracerebroventricular administration.

In one embodiment, the AAV particles may be delivered by direct injection into the brain. As a non-limiting example, the brain delivery may be by intrastriatal administration.

In one embodiment, the AAV particles may delivered to a subject using a device to deliver the AAV particles and a head fixation assembly. The head fixation assembly may be, but is not limited to, any of the head fixation assemblies sold by MRI interventions. As a non-limiting example, the head fixation assembly may be any of the assemblies described in U.S. Pat. Nos. 8,099,150, 8,548,569 and 9,031,636 and International Patent Publication Nos. WO201108495 and WO2014014585, the contents of each of which are incorporated by reference in their entireties. A head fixation assembly may be used in combination with an MRI compatible drill such as, but not limited to, the MRI compatible drills described in International Patent Publication No. WO2013181008 and US Patent Publication No. US20130325012, the contents of which are herein incorporated by reference in its entirety.

In one embodiment, the AAV particles may be delivered using a method, system and/or computer program for positioning apparatus to a target point on a subject to deliver the AAV particles. As a non-limiting example, the method, system and/or computer program may be the methods, systems and/or computer programs described in U.S. Pat. No. 8,340,743, the contents of which are herein incorporated by reference in its entirety. The method may include: determining a target point in the body and a reference point, wherein the target point and the reference point define a planned trajectory line (PTL) extending through each; determining a visualization plane, wherein the PTL intersects the visualization plane at a sighting point; mounting the guide device relative to the body to move with respect to the PTL, wherein the guide device does not intersect the visualization plane; determining a point of intersection (GPP) between the guide axis and the visualization plane; and aligning the GPP with the sighting point in the visualization plane.

In one embodiment, the AAV particles may be delivered using an MRI-guided device. Non-limiting examples of MRI-guided devices are described in U.S. Pat. Nos. 9,055,884, 9,042,958, 8,886,288, 8,768,433, 8,396,532, 8,369,930, 8,374,677 and 8,175,677 and US Patent Application No. US20140024927 the contents of each of which are herein incorporated by reference in their entireties. As a non-limiting example, the MRI-guided device may be able to provide data in real time such as those described in U.S. Pat. Nos. 8,886,288 and 8,768,433, the contents of each of which is herein incorporated by reference in its entirety. As another non-limiting example, the MRI-guided device or system may be used with a targeting cannula such as the systems described in U.S. Pat. Nos. 8,175,677 and 8,374,677, the contents of each of which are herein incorporated by reference in their entireties. As yet another non-limiting example, the MRI-guided device includes a trajectory guide frame for guiding an interventional device as described, for example, in U.S. Pat. No. 9,055,884 and US Patent Application No. US20140024927, the contents of each of which are herein incorporated by reference in their entireties.

In one embodiment the AAV particles may be delivered using an MRI-compatible tip assembly. Non-limiting examples of MRI-compatible tip assemblies are described in US Patent Publication No. US20140275980, the contents of which is herein incorporated by reference in its entirety.

In one embodiment, the AAV particles may be delivered using a cannula which is MRI-compatible. Non-limiting examples of MRI-compatible cannulas include those taught in International Patent Publication No. WO2011130107, the contents of which are herein incorporated by reference in its entirety.

In one embodiment, the AAV particles may be delivered using a catheter which is MRI-compatible. Non-limiting examples of MRI-compatible catheters include those taught in International Patent Publication No. WO2012116265, U.S. Pat. No. 8,825,133 and US Patent Publication No. US20140024909, the contents of each of which are herein incorporated by reference in their entireties.

In one embodiment, the AAV particles may be delivered using a device with an elongated tubular body and a diaphragm as described in US Patent Publication Nos. US20140276582 and US20140276614, the contents of each of which are herein incorporated by reference in their entireties.

In one embodiment, the AAV particles may be delivered using an MRI compatible localization and/or guidance system such as, but not limited to, those described in US Patent Publication Nos. US20150223905 and US20150230871, the contents of each of which are herein incorporated by reference in their entireties. As a non-limiting example, the MRI compatible localization and/or guidance systems may comprise a mount adapted for fixation to a patient, a targeting cannula with a lumen configured to attach to the mount so as to be able to controllably translate in at least three dimensions, and an elongate probe configured to snugly advance via slide and retract in the targeting cannula lumen, the elongate probe comprising at least one of a stimulation or recording electrode.

In one embodiment, the AAV particles may be delivered to a subject using a trajectory frame as described in US Patent Publication Nos. US20150031982 and US20140066750 and International Patent Publication Nos. WO2015057807 and WO2014039481, the contents of each of which are herein incorporated by reference in their entireties.

In one embodiment, the AAV particles may be delivered to a subject to improve and/or correct mitochondrial dysfunction.

In one embodiment, the AAV particles may be delivered to a subject to preserve neurons. The neurons may be primary and/or secondary sensor neurons.

In one embodiment, administration of the AAV particles may preserve and/or correct function in the sensory pathways.

In one embodiment, administration of the AAV particles may protect central pathways from degeneration. As a non-limiting example, the degeneration is later onset degeneration of auditory pathways.

### Dosing

The present invention provides methods of administering AAV particles in accordance with the invention to a subject in need thereof. AAV particle pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like. Compositions in accordance with the invention are typically formulated in unit dosage form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific payload employed; the specific composition employed; the age, body weight, general health, sex and diet of the patient; the time of administration, route of administration, and rate of excretion of the specific payload employed; the duration of the treatment; drugs used in combination or coincidental with the specific payload employed; and like factors well known in the medical arts.

In certain embodiments, AAV particle pharmaceutical compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. It will be understood that the above dosing concentrations may be converted to vg or viral genomes per kg or into total viral genomes administered by one of skill in the art.

As used herein, a “single unit dose” is a dose of any therapeutic administered in one dose/at one time/single route/single point of contact, i.e., single administration event. As used herein, a “total daily dose” is an amount given or prescribed in 24 hour period. It may be administered as a single unit dose. The viral particles may be formulated in buffer only or in a formulation described herein.

In one embodiment, delivery of the AAV particles described herein results in minimal serious adverse events (SAEs) as a result of the delivery of the AAV particles.

In one embodiment, a subject has had a low incidence of mild to moderate adverse events (AEs) near the time of the administration of the AAV particles. The subject may have had a low incidence of mild to moderate AEs within minutes (e.g., 1, 5, 10, 15, 20, 25, 30, 35, 40, 45, 50, 55 or 60 minutes), hours (1, 2, 3, 4, 5, 6, 7, 8, 9, 10, 11, 12, 13, 14, 15, 16, 17, 18, 19, 20, 21, 22, 23, or 24 hours) or days (1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days).

In one embodiment, delivery of AAV particles to cells of the central nervous system (e.g., parenchyma) may comprise a total dose between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG. As a non-limiting example, the total dose is 1×10¹³VG. As another non-limiting example, the total dose is 3×10¹³VG. As another non-limiting example, the total dose is 3.73×10¹⁰ VG. As another non-limiting example, the total dose is 1.9×10¹⁰ VG. As another non-limiting example, the total dose is 2.5×10¹¹ VG. As another non-limiting example, the total dose is 5×10¹¹ VG. As another non-limiting example, the total dose is 1×10¹² VG. As another non-limiting example, the total dose is 5×10¹² VG.

In one embodiment, delivery of AAV particles to cells of the central nervous system (e.g., parenchyma) may comprise a composition concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹² 4×10¹², 5×10¹², 6×10¹², 7×10¹², 8×10¹², 9×10¹², 1×10¹³, 2×10¹³, 3×10¹³, 4×10¹³, 5×10¹³ 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴ 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵ or 1×10¹⁶ VG/mL. In one embodiment, the delivery comprises a composition concentration of 1×10¹³ VG/mL. In one embodiment, the delivery comprises a composition concentration of 3×10¹² VG/mL.

In one embodiment, delivery of AAV particles to cells of the central nervous system (e.g., parenchyma) may comprise a composition concentration between about 1×10⁶ VG/uL and about 1×10¹⁶ VG/uL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹² 4×10¹², 5×10¹², 6×10¹², 7×10¹², 8×10¹², 9×10¹², 1×10¹³, 2×10¹³, 3×10¹³, 4×10¹³, 5×10¹³ 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴ 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵ or 1×10¹⁶ VG/uL. In one embodiment, the delivery comprises a composition concentration of 1×10¹³ VG/uL. In one embodiment, the delivery comprises a composition concentration of 3×10¹² VG/uL. In one embodiment, the delivery comprises a composition concentration of 1.9×10¹⁰ VG/10 uL. In one embodiment, the delivery comprises a composition concentration of 2.5×10¹¹ VG/100 uL. In one embodiment, the delivery comprises a composition concentration of 5×10¹¹ VG/100 uL.

In one embodiment, the dosage delivered to a subject may take into account the amount of backflow of the substance. As a non-limiting example, the method for determining the backflow of a substance or fluid along a track of a delivery device is described in U.S. Pat. Nos. 7,742,630, 7,715,902 and European Publication No. EP1788498, the contents of each of which is herein incorporated by reference in their entireties. As a non-limiting example, a method of reducing the amount of backflow which is described in US Patent Publication No. US20140243783, the contents of which are herein incorporated by reference in its entirety, may be used to reduce the backflow from the administration of composition comprising AAV particles described herein.

### Combinations

The AAV particles may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body.

In one embodiment, the AAV particles may be delivered via intracerebroventricular (ICV) and/or intrathecal (IT) infusion and therapeutic agent may also be delivered to a subject via intravascular limb infusion in order to deliver the therapeutic agent to the skeletal muscle. Delivery of adeno-associate virus by intravascular limb infusion is described by Gruntman and Flotte (Human Gene Therapy Clinical Development, Vol. 26(3), 2015 159-164; the contents of which is herein incorporated by reference in its entirety).

### Delivery to Cells

The present disclosure provides a method of delivering to a cell or tissue any of the above-described AAV particle, comprising contacting the cell or tissue with said AAV particle or contacting the cell or tissue with a particle comprising said AAV particle, or contacting the cell or tissue with any of the described compositions, including pharmaceutical compositions. The method of delivering the AAV particle to a cell or tissue can be accomplished in vitro, ex vivo, or in vivo.

### Delivery to Subjects

The present disclosure additionally provides a method of delivering to a subject, including a mammalian subject, any of the above-described AAV particle comprising administering to the subject said AAV particle, or administering to the subject a particle comprising said AAV particle, or administering to the subject any of the described compositions, including pharmaceutical compositions.

### Measurement of Expression

Expression of payloads from viral genomes may be determined using various methods known in the art such as, but not limited to immunochemistry (e.g., IHC) or in situ hybridization (ISH). In one embodiment, transgenes delivered in different AAV capsids may have different expression levels in Dorsal Root Ganglion (DRG).

### CNS Diseases

The present disclosure provides a method for treating a disease, disorder and/or condition in a mammalian subject, including a human subject, comprising administering to the subject any of the viral particles e.g., AAV, AAV particle or AAV genomes described herein (i.e., viral genomes or “VG”) or administering to the subject a particle comprising said AAV particle or AAV genome, or administering to the subject any of the described compositions, including pharmaceutical compositions. In one embodiment, the disease, disorder and/or condition is a neurological disease, disorder and/or condition. The CNS diseases may be diseases that affect any component of the brain (including the cerebral hemispheres, diencephalon, brain stem, and cerebellum) or the spinal cord.

In some embodiments, AAV particles of the present invention, through delivery of a function payload that is a therapeutic product that can modulate the level or function of a gene product in the CNS, may be used to treat a neurodegenerative diseases and/or diseases or disorders that are characteristic with neurodegeneration, neuromuscular diseases, lysosomal diseases, trauma, bone marrow injuries, pain (including neuropathic pain), cancers of the nervous system, demyelinating diseases, autoimmune diseases of the nervous system, neurotoxic syndromes, sleeping disorders genetic brain disorders and developmental CNS disorders. A functional payload may alleviate or reduce symptoms that result from abnormal level and/or function of a gene product (e.g., an absence or defect in a protein) in a subject in need thereof or that otherwise confers a benefit to a CNS disorder in a subject in need thereof.

As non-limiting examples, therapeutic products delivered by AAV particles of the present invention may include, but are not limited to, growth and trophic factors, cytokines, hormones, neurotransmitters, enzymes, anti-apoptotic factors, angiogenic factors, and any protein known to be mutated in pathological disorders such as the “survival of motor neuron” protein (SMN); antisense RNA or RNAi targeting messenger RNAs coding for proteins having a therapeutic interest in any of CNS diseases discussed herein; or microRNAs that function in gene silencing and post-transcriptionally regulation of gene expression in the CNS (e.g., brain specific Mir-128a, See Adlakha and Saini, Molecular cancer, 2014, 13:33).

The growth and trophic factors may include, but are not limited to brain-derived growth factor (BDNF), epidermal growth factor (EGF), basic Fibroblast growth factor (bFGF), Ciliary neurotrophic factor (CNTF), corticotropin-releasing factor (CRF), Glial cell line derived growth factor (GDNF), Insulin-like growth factor-1 (IGF-1), nerve growth factor (NGF), neurotrophin-3 (NT-3), neurotrophin-4 (NT-4), and vascular endothelial growth factor (VEGF). Cytokines may include interleukin-10 (IL-10), interleukin-6, Interleukin-8, chemokine CXCL12 (SDF-1), TGF-beta, and Growth and differentiation factor (GDF-1/10).

In some embodiments, the neurological disorders may be neurodegenerative disorders including, but not limited to, Alzheimer's Diseases (AD); Amyotrophic lateral sclerosis (ALS); Creutzfeldt-Jakob Disease; Huntingtin's disease (HD); Friedreich's ataxia (FA); Parkinson Disease (PD); Multiple System Atrophy (MSA); Spinal Muscular Atrophy (SMA), Multiple Sclerosis (MS); Primary progressive aphasia; Progressive supranuclear palsy; Dementia; Brain Cancer, Degenerative Nerve Diseases, Encephalitis, Epilepsy, Genetic Brain Disorders that cause neurodegeneration, Retinitis pigmentosa (RP), Head and Brain Malformations, Hydrocephalus, Stroke, Prion disease, Infantile neuronal ceroid lipofuscinosis (INCL) (a neurodegenerative disease of children caused by a deficiency in the lysosomal enzyme palmitoyl protein thioesterase-1 (PPT1)), and others.

In some embodiments, AAV particles of the present invention may be used to treat diseases that are associated with impairments of the growth and development of the CNS, i.e., neurodevelopmental disorders. In some aspects, such neurodevelopmental disorders may be caused by genetic mutations, including but not limited to, Fragile X syndrome (caused by mutations in FMR1 gene), Down syndrome (caused by trisomy of chromosome 21), Rett syndrome, Williams syndrome, Angelman syndrome, Smith-Magenis syndrome, ATR-X syndrome, Barth syndrome, Immune dysfunction and/or infectious diseases during infancy such as Sydenham's chorea, Schizophrenia Congenital toxoplasmosis, Congenital rubella syndrome, Metabolic disorders such as diabetes mellitus and phenylketonuria; nutritional defects and/or brain trauma, Autism and autism spectrum.

In some embodiments, AAV particles of the present invention, may be used to treat a tumor in the CNS, including but not limited to, acoustic neuroma, Astrocytoma (Grades I, II, III and IV), Chordoma, CNS Lymphoma, Craniopharyngioma, Gliomas (e.g., brain stem glioma, ependymoma, optical nerve glioma, subependymoma), Medulloblastoma, Meningioma, Metastatic brain tumors, Oligodendroglioma, Pituitary Tumors, Primitive neuroectodermal (PNET), and Schwannoma.

In some embodiments, the neurological disorders may be functional neurological disorders with motor and/or sensory symptoms which have neurological origin in the CNS. As non-limiting examples, functional neurological disorders may be chronic pain, seizures, speech problems, involuntary movements, and sleep disturbances.

In some embodiments, the neurological disorders may be white matter disorders (a group of diseases that affects nerve fibers in the CNS) including but not limited to, Pelizaeus-Merzbacher disease, Hypomyelination with atrophy of basal ganglia and cerebellum, Aicardi-Goutières syndrome, Megalencephalic leukoencephalopathy with subcortical cysts, Congenital muscular dystrophies, Myotonic dystrophy, Wilson disease, Lowe syndrome, Sjögren-Larsson syndrome, PIBD or Tay syndrome, Cockayne's disease, erebrotendinous xanthomatosis, Zellweger syndrome, Neonatal adrenoleukodystrophy, Infantile Refsum disease, Zellweger-like syndrome, Pseudo-Zellweger syndrome, Pseudo-neonatal adrenoleukodystrophy, Bifunctional protein deficiency, X-linked adrenoleukodystrophy and adrenomyeloneuropathy and Refsum disease.

In some embodiments, the neurological disorders may be lysosomal storage disorders (LSDs) caused by the inability of cells in the CNS to break down metabolic end products, including but not limited to Niemann-Pick disease (a LSD resulting from inherited deficiency in acid sphingomyelinase (ASM); Metachromatic leukodystrophy (MLD) (a LSD characterized by accumulation of sulfatides in glial cells and neurons, the result of an inherited deficiency of arylsulfatase A (ARSA)); Globoid-cell leukodystrophy (GLD) (a LSD caused by mutations in galactosylceramidase); Fabry disease (a LSD caused by mutations in the alpha-galactosidase A (GLA) gene); Gaucher disease (caused by mutations in the beta-glucocerebrosidase (GBA) gene); GM1/GM2 gangliosidosis; Mucopolysaccharidoses disorder; Pompe disease; and Neuronal ceroid lipofuscinosis.

In one embodiment, the neurological disease, disorder and/or condition is Parkinson's disease. In one embodiment, the polynucleotide used to treat Parkinson's disease comprises any one of SEQ ID NOs 1-93 wherein the payload is replaced by AADC or any other payload known in the art for treating Parkinson's disease. As a non-limiting example, the payload may be a sequence such as NM_001082971.1 (GI: 132814447), NM_000790.3 (GI: 132814459), NM_001242886.1 (GI: 338968913), NM_001242887.1 (GI: 338968916), NM_001242888.1 (GI: 338968918), NM_001242889.1 (GI: 338968920), NM_001242890.1 (GI: 338968922) and fragment or variants thereof.

In another embodiment, the neurological disease, disorder and/or condition is Friedreich's Ataxia. In one embodiment, the delivery of the AAV particles may halt or slow the disease progression of Friedreich's Ataxia by 10%, 20%, 30%, 40%, 50%, 60%, 70%, 80%, 90%, 95% or more than 95% using a known analysis method and comparator group for Friedreich's Ataxia. As a non-limiting example, the delivery of the AAV particles may halt or slow progression of Friedreich's Ataxia progression as measured by mFARS/SARA by 50% relative to a comparator group. In one embodiment the polynucleotide used to treat Friedreich's Ataxia comprises any one of SEQ ID NOs 1-93 wherein the payload is replaced by Frataxin or any other payload known in the art for treating Friedreich's Ataxia. As a non-limiting example, the payload may be a sequence such as NM_000144.4 (GI: 239787167), NM_181425.2 (GI: 239787185), NM_001161706.1 (GI: 239787197) and fragment or variants thereof.

In another embodiment, the neurological disease, disorder and/or condition is Amyotrophic lateral sclerosis (ALS). In one embodiment, the delivery of the AAV particles may halt or slow the disease progression of ALS by 10%, 20%, 30%, 40%, 50%, 60%, 70%, 80%, 90%, 95% or more than 95% using a known analysis method and comparator group for ALS. In one embodiment the polynucleotide used to treat ALS comprises any one of SEQ ID NOs 1-93 wherein the payload is replaced by an shRNA, miRNA, siRNA, RNAi for SOD1 or any other payload known in the art for treating ALS.

In another embodiment, the neurological disease, disorder and/or condition is Huntington's disease. In one embodiment, the delivery of the AAV particles may halt or slow the disease progression of Huntington's disease by 10%, 20%, 30%, 40%, 50%, 60%, 70%, 80%, 90%, 95% or more than 95% using a known analysis method and comparator group for Huntington's disease. In one embodiment the polynucleotide used to treat Huntington's disease comprises any one of SEQ ID NOs 1-93 wherein the payload is replaced by an shRNA, miRNA, siRNA, RNAi for Htt or any other payload known in the art for treating Huntington's disease.

In another embodiment, the neurological disease, disorder or condition is spinal muscular atrophy (SMA). In one embodiment, the delivery of the AAV particles may halt or slow the disease progression of SMA by 10%, 20%, 30%, 40%, 50%, 60%, 70%, 80%, 90%, 95% or more than 95% using a known analysis method and comparator group for SMA. In one embodiment the polynucleotide used to treat SMA comprises any one of SEQ ID NOs 1-93 wherein the payload is replaced by SMN or any other payload known in the art for treating SMA. As a non-limiting example, the payload may be a sequence such as NM_001297715.1 (GI: 663070993), NM_000344.3 (GI: 196115055), NM_022874.2 (GI: 196115040) and fragment or variants thereof.

In one embodiment, the AAV particle encoding a payload may increase the amount of protein encoded by the transgene by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 97%, 99% or more than 100%.

In one embodiment, the AAV particle encoding a payload may increase the amount of protein encoded by the transgene by 1-5%, 1-10%, 1-15%, 1-20%, 5-10%, 5-15%, 5-20%, 5-25%, 10-20%, 10-30%, 15-35%, 20-40%, 20-50%, 30-50%, 30-60%, 40-60%, 40-70%, 50-60%, 50-70%, 60-80%, 60-90%, 70-80%, 70-90%, 80-90%, 80-99% or 90-100%.

### Definitions

At various places in the present specification, substituents of compounds of the present disclosure are disclosed in groups or in ranges. It is specifically intended that the present disclosure include each and every individual sub-combination of the members of such groups and ranges. The following is a non-limiting list of term definitions.

Adeno-associated virus: The term “adeno-associated virus” or “AAV” as used herein refers to members of the dependovirus genus comprising any particle, sequence, gene, protein, or component derived therefrom. The term “AAV particle” as used herein comprises a capsid and a polynucleotide referred to as the AAV genome or viral genome (VG). The AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted.

Activity: As used herein, the term “activity” refers to the condition in which things are happening or being done. Compositions of the invention may have activity and this activity may involve one or more biological events.

Administered in combination: As used herein, the term “administered in combination” or “combined administration” refers to simultaneous exposure to two or more agents (e.g., AAV) administered at the same time or within an interval such that the subject is at some point in time simultaneously exposed to both and/or such that there may be an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatus in administration. In some embodiments, the administration of individual doses of one or more compounds and/or compositions of the present invention, as described herein, are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, mean that the moieties are physically associated or connected with one another, either directly or via one or more additional moieties that serve as linking agents, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Biomolecule: As used herein, the term “biomolecule” is any natural molecule which is amino acid-based, nucleic acid-based, carbohydrate-based or lipid-based, and the like.

Biologically active: As used herein, the phrase “biologically active” refers to a characteristic of any substance (e.g., an AAV) that has activity in or on a biological system and/or organism. For instance, a substance that, when administered to an organism, has a biological effect on that organism, is considered to be biologically active. In particular embodiments, a compounds and/or compositions of the present invention may be considered biologically active if even a portion of is biologically active or mimics an activity considered to biologically relevant.

Biological system: As used herein, the term “biological system” refers to a group of organs, tissues, cells, intracellular components, proteins, nucleic acids, molecules (including, but not limited to biomolecules) that function together to perform a certain biological task within cellular membranes, cellular compartments, cells, tissues, organs, organ systems, multicellular organisms, or any biological entity. In some embodiments, biological systems are cell signaling pathways comprising intracellular and/or extracellular cell signaling biomolecules. In some embodiments, biological systems comprise growth factor signaling events within the extracellular/cellular matrix and/or cellular niches.

Central Nervous System or CNS: As used herein, “Central Nervous System” or “CNS” refers to one of the two major subdivisions of the nervous system, which in vertebrates includes of the brain and spinal cord. The central nervous system coordinates the activity of the entire nervous system.

Cervical Region: As used herein, “Cervical Region” refers to the region of the spinal cord comprising the cervical vertebrae C1, C2, C3, C4, C5, C6, C7, and C9.

CNS tissue: As used herein, “CNS tissue” or “CNS tissues” refers to the tissues of the central nervous system, which in vertebrates, include the brain and spinal cord and sub-structures thereof.

CNS structures: As used herein, “CNS structures” refers to structures of the central nervous system and sub-structures thereof. Non-limiting examples of structures in the spinal cord may include, ventral horn, dorsal horn, white matter, and nervous system pathways or nuclei within. Non limiting examples of structures in the brain include, forebrain, midbrain, hindbrain, diencephalon, telencephalon, myelencepphalon, metencephalon, mesencephalon, prosencephalon, rhombencephalon, cortices, frontal lobe, parietal lobe, temporal lobe, occipital lobe, cerebrum, thalamus, hypothalamus, tectum, tegmentum, cerebellum, pons, medulla, amygdala, hippocampus, basal ganglia, corpus callosum, pituitary gland, ventricles and sub-structures thereof.

CNS Cells: As used herein, “CNS Cells” refers to cells of the central nervous system and sub-structures thereof. Non-limiting examples of CNS cells include, neurons and sub-types thereof, glia, microglia, oligodendrocytes, ependymal cells and astrocytes. Non-limiting examples of neurons include sensory neurons, motor neurons, interneurons, unipolar cells, bipolar cells, multipolar cells, psuedounipolar cells, pyramidal cells, basket cells, stellate cells, purkinje cells, betz cells, amacrine cells, granule cell, ovoid cell, medium aspiny neurons and large aspiny neurons.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Composition: As used herein, the term “composition” comprises an AAV polynucleotide, AAV genome or AAV particle and at least one excipient.

Compound: As used herein, the term “compound,” refers to a distinct chemical entity. In some embodiments, a particular compound may exist in one or more isomeric or isotopic forms (including, but not limited to stereoisomers, geometric isomers and isotopes). In some embodiments, a compound is provided or utilized in only a single such form. In some embodiments, a compound is provided or utilized as a mixture of two or more such forms (including, but not limited to a racemic mixture of stereoisomers). Those of skill in the art appreciate that some compounds exist in different such forms, show different properties and/or activities (including, but not limited to biological activities). In such cases it is within the ordinary skill of those in the art to select or avoid particular forms of the compound for use in accordance with the present invention. For example, compounds that contain asymmetrically substituted carbon atoms can be isolated in optically active or racemic forms. Methods on how to prepare optically active forms from optically active starting materials are known in the art, such as by resolution of racemic mixtures or by stereoselective synthesis.

Conserved: As used herein, the term “conserved” refers to nucleotides or amino acid residues of polynucleotide or polypeptide sequences, respectively, that are those that occur unaltered in the same position of two or more sequences being compared. Nucleotides or amino acids that are relatively conserved are those that are conserved among more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

In one embodiment, conserved sequences are not contiguous. Those skilled in the art are able to appreciate how to achieve alignment when gaps in contiguous alignment are present between sequences, and to align corresponding residues not withstanding insertions or deletions present.

Delivery: As used herein, “delivery” refers to the act or manner of delivering a parvovirus e.g., AAV compound, substance, entity, moiety, cargo or payload to a target. Such target may be a cell, tissue, organ, organism, or system (whether biological or production).

Delivery Agent: As used herein, “delivery agent” refers to any agent which facilitates, at least in part, the delivery of one or more substances (including, but not limited to a compounds and/or compositions of the present invention, e.g., viral particles or AAV vectors) to targeted cells.

Destabilized: As used herein, the term “destable,” “destabilize,” or “destabilizing region” means a region or molecule that is less stable than a starting, reference, wild-type or native form of the same region or molecule.

Detectable label: As used herein, “detectable label” refers to one or more markers, signals, or moieties which are attached, incorporated or associated with another entity, which markers, signals or moieties are readily detected by methods known in the art including radiography, fluorescence, chemiluminescence, enzymatic activity, absorbance, immunological detection and the like. Detectable labels may include radioisotopes, fluorophores, chromophores, enzymes, dyes, metal ions, ligands, biotin, avidin, streptavidin and haptens, quantum dots, polyhistidine tags, myc tags, flag tags, human influenza hemagglutinin (HA) tags and the like. Detectable labels may be located at any position in the entity with which they are attached, incorporated or associated. For example, when attached, incorporated in or associated with a peptide or protein, they may be within the amino acids, the peptides, or proteins, or located at the N- or C-termini.

Effective amount: As used herein, the term “effective amount” of an agent is that amount sufficient to effect beneficial or desired results, for example, upon single or multiple dose administration to a subject or a cell, in curing, alleviating, relieving or improving one or more symptoms of a disorder and, as such, an “effective amount” depends upon the context in which it is being applied. For example, in the context of administering an agent that treats ALS, an effective amount of an agent is, for example, an amount sufficient to achieve treatment, as defined herein, of ALS, as compared to the response obtained without administration of the agent.

Engineered: As used herein, embodiments of the invention are “engineered” when they are designed to have a feature or property, whether structural or chemical, that varies from a starting point, wild-type or native molecule. Thus, engineered agents or entities are those whose design and/or production include an act of the hand of man.

Epitope: As used herein, an “epitope” refers to a surface or region on a molecule that is capable of interacting with a biomolecule. For example a protein may contain one or more amino acids, e.g., an epitope, which interacts with an antibody, e.g., a biomolecule. In some embodiments, when referring to a protein or protein module, an epitope may comprise a linear stretch of amino acids or a three dimensional structure formed by folded amino acid chains.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Feature: As used herein, a “feature” refers to a characteristic, a property, or a distinctive element.

Formulation: As used herein, a “formulation” includes at least a compound and/or composition of the present invention (e.g., a vector, AAV particle, etc.) and a delivery agent.

Fragment: A “fragment,” as used herein, refers to a contiguous portion of a whole. For example, fragments of proteins may comprise polypeptides obtained by digesting full-length protein isolated from cultured cells. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids.

Functional: As used herein, a “functional” biological molecule is a biological entity with a structure and in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

High Cervical Region: As used herein, the term “high cervical region” refers to the region of the spinal cord comprising the cervical vertebrae C1, C2, C3 and C4 or any subset thereof.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is typically determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids. In many embodiments, homologous protein may show a large overall degree of homology and a high degree of homology over at least one short stretch of at least 3, 4, 5, 6, 7, 8, 9, 10, 11, 12, 13, 14, 15, 16, 17, 18, 19, 20, 25, 30, 35, 40, 45, 50 or more amino acids. In many embodiments, homologous proteins share one or more characteristic sequence elements. As used herein, the term “characteristic sequence element” refers to a motif present in related proteins. In some embodiments, the presence of such motifs correlates with a particular activity (such as biological activity).

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, may be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference in its entirety. For example, the percent identity between two nucleotide sequences can be determined, for example using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference in its entirety. Techniques for determining identity are codified in publicly available computer programs. Computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product may be RNA transcribed from the gene (e.g. mRNA) or a polypeptide translated from mRNA transcribed from the gene. Typically a reduction in the level of mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” is synonymous with “separated”, but carries with it the inference separation was carried out by the hand of man. In one embodiment, an isolated substance or entity is one that has been separated from at least some of the components with which it was previously associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art. In some embodiments, isolation of a substance or entity includes disruption of chemical associations and/or bonds. In some embodiments, isolation includes only the separation from components with which the isolated substance or entity was previously combined and does not include such disruption.

Lumbar Region: As used herein, the term “lumbar region” refers to the region of the spinal cord comprising the lumbar vertebrae L1, L2, L3, L4, and L5.

Modified: As used herein, the term “modified” refers to a changed state or structure of a molecule or entity as compared with a parent or reference molecule or entity. Molecules may be modified in many ways including chemically, structurally, and functionally. In some embodiments, compounds and/or compositions of the present invention are modified by the introduction of non-natural amino acids, or non-natural nucleotides.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids). In embodiments wherein mutations comprise the addition and/or substitution of amino acids and/or nucleotides, such additions and/or substitutions may comprise 1 or more amino acid and/or nucleotide residues and may include modified amino acids and/or nucleotides.

Naturally occurring: As used herein, “naturally occurring” or “wild-type” means existing in nature without artificial aid, or involvement of the hand of man.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic acid: As used herein, the term “nucleic acid”, “polynucleotide” and ‘oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA.

Off-target: As used herein, “off target” refers to any unintended effect on any one or more target, gene and/or cellular transcript.

Operably linked: As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained (e.g., licensed) professional for a particular disease or condition.

Payload: As used herein, “payload” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid.

Payload construct: As used herein, “payload construct” is one or more polynucleotide regions encoding or comprising a payload that is flanked on one or both sides by an inverted terminal repeat (ITR) sequence. The payload construct is a template that is replicated in a viral production cell to produce a viral genome.

Payload construct vector: As used herein, “payload construct vector” is a vector encoding or comprising a payload construct, and regulatory regions for replication and expression in bacterial cells. The payload construct vector may also comprise component for viral expression in a viral replication cell

Peptide: As used herein, the term “peptide” refers to a chain of amino acids that is less than or equal to about 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: As used herein, the term “pharmaceutically acceptable excipient,” as used herein, refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety. Pharmaceutically acceptable solvate: The term “pharmaceutically acceptable solvate,” as used herein, refers to a crystalline form of a compound wherein molecules of a suitable solvent are incorporated in the crystal lattice. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

Pharmaceutical Composition: As used herein, the term “pharmaceutical composition” or pharmaceutically acceptable composition” comprises an AAV polynucleotides, AAV genomes or AAV particle and one or more pharmaceutically acceptable excipients.

Pharmacokinetic: As used herein, “pharmacokinetic” refers to any one or more properties of a molecule or compound as it relates to the determination of the fate of substances administered to living organisms. Pharmacokinetics are divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand, replicate or increase or cause to grow, expand, replicate or increase. “Proliferative” means having the ability to proliferate. “Anti-proliferative” means having properties counter to or in opposition to proliferative properties.

Protein of interest: As used herein, the terms “proteins of interest” or “desired proteins” include those provided herein and fragments, mutants, variants, and alterations thereof.

Purified: As used herein, the term “purify” means to make substantially pure or clear from unwanted components, material defilement, admixture or imperfection. “Purified” refers to the state of being pure. “Purification” refers to the process of making pure.

Region: As used herein, the term “region” refers to a zone or general area. In some embodiments, when referring to a protein or protein module, a region may comprise a linear sequence of amino acids along the protein or protein module or may comprise a three dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In some embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus, but does not include the 3′ terminus. In some embodiments, 3′ regions may comprise any length of nucleic acids, which include the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interference: As used herein, the term “RNA interference” or “RNAi” refers to a sequence specific regulatory mechanism mediated by RNA molecules which results in the inhibition or interference or “silencing” of the expression of a corresponding protein-coding gene.

Sacral Region: As used herein, the term “sacral region” refers to the region of the spinal cord comprising the sacral vertebrae S1, S2, S3, S4, and S5.

Sample: As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample is or comprises a medium, such as a nutrient broth or gel, which may contain cellular components, such as proteins or nucleic acid molecule. In some embodiments, a “primary” sample is an aliquot of the source. In some embodiments, a primary sample is subjected to one or more processing (e.g., separation, purification, etc.) steps to prepare a sample for analysis or other use.

Self-complementary viral particle: As used herein, a “self-complementary viral particle” is a particle comprised of at least two components, a protein capsid and a polynucleotide sequence encoding a self-complementary genome enclosed within the capsid.

Sense strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the siRNA strand.

Signal Sequences: As used herein, the phrase “signal sequences” refers to a sequence which can direct the transport or localization.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Small/short interfering RNA: As used herein, the term “small/short interfering RNA” or “siRNA” refers to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

Split dose: As used herein, a “split dose” is the division of single unit dose or total daily dose into two or more doses.

Stable: As used herein “stable” refers to a compound or entity that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable. In some embodiments, stability is measured relative to an absolute value. In some embodiments, stability is measured relative to a reference compound or entity.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term typically means within about 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present invention may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Thoracic Region: As used herein, a “thoracic region” refers to a region of the spinal cord comprising the thoracic vertebrae T1, T2, T3, T4, T5, T6, T7, T8, T9, T10, T11, and T12.

Total daily dose: As used herein, a “total daily dose” is an amount given or prescribed in a 24 hour period. It may be administered as a single unit dose.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild-type or native form of a biomolecule or entity. Molecules or entities may undergo a series of modifications whereby each modified product may serve as the “unmodified” starting molecule or entity for a subsequent modification.

Vector: As used herein, a “vector” is any molecule or moiety which transports, transduces or otherwise acts as a carrier of a heterologous molecule. Vectors of the present invention may be produced recombinantly and may be based on and/or may comprise adeno-associated virus (AAV) parent or reference sequence. Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral construct vector: As used herein, a “viral construct vector” is a vector which comprises one or more polynucleotide regions encoding or comprising Rep and or Cap protein. A viral construct vector may also comprise one or more polynucleotide region encoding or comprising components for viral expression in a viral replication cell.

Viral genome: As used herein, a “viral genome” is a polynucleotide encoding at least one inverted terminal repeat (ITR), at least one regulatory sequence, and at least one payload. The viral genome is derived by replication of a payload construct from the payload construct vector. A viral genome encodes at least one copy of the payload construct.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

### EXAMPLES

### Example 1. Design of Payload Constructs

Payload constructs were designed to comprise at a minimum a nucleic acid sequence encoding a frataxin protein.

Once designed, the sequence was engineered or synthesized or inserted in a plasmid or vector and administered to a cell or organism. Suitable plasmids or vectors were any which transduce or transfect the target cell.

Adeno-associated viral vectors (AAV), viral particles or entire viruses may be used.

Administration resulted in the processing of the payload construct to generate the frataxin protein which alters the etiology of the disease, in this case Friedriech's Ataxia.

AAV constructs were designed and built using standard molecular cloning techniques. FXN-tag transgenes were cloned into either pAAVss, pAAVsc, or pcDNA3.1 plasmid and the resulting clones were further sequenced to confirm the correctness of all elements such as ITRs, promoters, and tags.

In one non-limiting example, plasmids containing a payload construct are described herein and some are described in Table 3. These payload constructs in Table 3 may comprise a pCDNA3.1, pAAVss, or pAAVsc vectors and may comprise the following components: a CMV, CB6, CB7, PGK, GFAP, hSYN, mCMVe-hEF1p, SV40, CBA or FXN promoter; an SV40 or MVM/CBA intron; a full or partial Kozak sequence; a FXN (Frataxin), CS (citrate synthase), RPL (ribosomal protein), SOD2 (superoxide dismutase), or AH (aconitate hydratase) signal peptide, also known as a mitochondrial targeting sequence (MTS); a cmyc, flag, cmycflag3, 3flag, 3flagcmyc, HA long or HA short tag; a SV40, rabbit beta-globin, or bGH poly (A) signal, 3′ and/or 5′ ITR sequences derived from any AAV genome comprising a partial and/or wild type sequence; and either wild type Frataxin or codon optimized Frataxin.

TABLE 3
AAV constructs

| | | | | 0.5 | Signal | | | | | | SEQ |
|---|---|---|---|---|---|---|---|---|---|---|---|
| Vector | Promoter | Intron | Kozak | Kozak | Peptide | Payload | Tag | 5′ITR | Poly(A) | 3′ITR | ID NO |
| pCDNA3.1(+) | CMV | N/A | − | − | FXN | FXN | cmyc3flag | − | bGH | − | 1 |
| pCDNA3.1(+) | CMV | N/A | − | − | FXN | FXN | — | − | bGH | − | 2 |
| pCDNA3.1(+) | CMV | N/A | − | − | FXN | FXN | 3flag | − | bGH | − | 3 |
| pCDNA3.1(+) | CMV | N/A | − | − | FXN | FXN | 3flagcmyc | − | bGH | − | 4 |
| pCDNA3.1(+) | CMV | N/A | − | − | FXN | FXN | cmyc | − | bGH | − | 5 |
| pCDNA3.1(+) | CMV | N/A | − | − | FXN | FXN | HA(L) | − | bGH | − | 6 |
| pCDNA3.1(+) | CMV | N/A | − | − | FXN | FXN | HA(S) | − | bGH | − | 7 |
| pCDNA3.1(+) | CMV | N/A | + | − | CS | FXN | — | − | bGH | − | 8 |
| pCDNA3.1(+) | CMV | N/A | − | + | FXN | FXN | — | − | bGH | − | 9 |
| pAAVss | CB6 | N/A | − | + | FXN | FXN | — | + | SV40 | + | 10 |
| pAAVss | CB6 | MVM/CBA | − | + | FXN | FXN | — | + | SV40 | + | 11 |
| pAAVss | CB6 | SV40 | − | − | FXN | FXN | — | + | SV40 | + | 12 |
| pAAVss | CB6 | SV40 | − | − | FXN | CodOp | — | + | SV40 | + | 13 |
| | | | | | | FXN | | | | | |
| pAAVss | CB6 | SV40 | − | − | FXN | CodOp | — | + | SV40 | + | 14 |
| | | | | | | FXN | | | | | |
| pAAVss | CB6 | SV40 | − | − | FXN | CodOp | — | + | SV40 | + | 15 |
| | | | | | | FXN | | | | | |
| pAAVss | CB6 | SV40 | − | − | FXN | CodOp | — | + | SV40 | + | 16 |
| | | | | | | FXN | | | | | |
| pAAVss | CB6 | SV40 | − | − | FXN | CodOp | — | + | SV40 | + | 17 |
| | | | | | | FXN | | | | | |
| pAAVss | CB6 | SV40 | − | − | FXN | CodOp | — | + | SV40 | + | 18 |
| | | | | | | FXN | | | | | |
| pAAVss | CB6 | SV40 | − | − | FXN | CodOp | — | + | SV40 | + | 19 |
| | | | | | | FXN | | | | | |
| pAAVss | CB6 | SV40 | − | − | FXN | CodOp | — | + | SV40 | + | 20 |
| | | | | | | FXN | | | | | |
| pAAVss | CB6 | SV40 | − | − | FXN | CodOp | — | + | SV40 | + | 21 |
| | | | | | | FXN | | | | | |
| pAAVss | CB6 | SV40 | − | − | FXN | CodOp | — | + | SV40 | + | 22 |
| | | | | | | FXN | | | | | |
| pAAVss | CB6 | SV40 | − | − | FXN | CodOp | — | + | SV40 | + | 23 |
| | | | | | | FXN | | | | | |
| pAAVss | CB6 | SV40 | − | − | FXN | CodOp | — | + | SV40 | + | 24 |
| | | | | | | FXN | | | | | |
| pAAVss | CB6 | SV40 | + | − | AH | FXN | — | + | SV40 | + | 25 |
| pAAVss | CB6 | SV40 | + | − | CS | FXN | — | + | SV40 | + | 26 |
| pAAVss | CB6 | SV40 | + | − | CS | FXN | HA(L) | + | SV40 | + | 27 |
| pAAVss | CB6 | SV40 | + | − | CS | FXN | HA(S) | + | SV40 | + | 28 |
| pAAVss | CB6 | SV40 | − | + | FXN | FXN | — | + | SV40 | + | 29 |
| pAAVss | CB6 | SV40 | − | + | FXN | CodOp | — | + | SV40 | + | 30 |
| | | | | | | FXN | | | | | |
| pAAVss | CB6 | SV40 | − | + | FXN | CodOp | — | + | SV40 | + | 31 |
| | | | | | | FXN | | | | | |
| pAAVss | CB6 | SV40 | − | + | FXN | CodOp | — | + | SV40 | + | 32 |
| | | | | | | FXN | | | | | |
| pAAVss | CB6 | SV40 | − | + | FXN | CodOp | — | + | SV40 | + | 33 |
| | | | | | | FXN | | | | | |
| pAAVss | CB6 | SV40 | − | + | FXN | CodOp | — | + | SV40 | + | 34 |
| | | | | | | FXN | | | | | |
| pAAVss | CB6 | SV40 | − | + | FXN | CodOp | — | + | SV40 | + | 35 |
| | | | | | | FXN | | | | | |
| pAAVss | CB6 | SV40 | − | + | FXN | CodOp | — | + | SV40 | + | 36 |
| | | | | | | FXN | | | | | |
| pAAVss | CB6 | SV40 | + | − | RPL | FXN | — | + | SV40 | + | 37 |
| pAAVss | CB6 | SV40 | + | − | RPL | CodOp | — | + | SV40 | + | 38 |
| | | | | | | FXN | | | | | |
| pAAVss | CB6 | SV40 | − | + | SOD2 | CodOp | — | + | SV40 | + | 39 |
| | | | | | | FXN | | | | | |
| pAAVss | CB7 | SV40 | − | − | FXN | FXN | — | + | SV40 | + | 40 |
| pAAVss | CMV | N/A | − | + | FXN | FXN | — | + | SV40 | + | 41 |
| pAAVss | CMV | SV40 | − | − | FXN | FXN | — | + | SV40 | + | 42 |
| pAAVss | CMV | SV40 | − | − | FXN | CodOp | — | + | SV40 | + | 43 |
| | | | | | | FXN | | | | | |
| pAAVss | CMV | SV40 | − | − | FXN | CodOp | — | + | SV40 | + | 44 |
| | | | | | | FXN | | | | | |
| pAAVss | CMV | SV40 | − | − | FXN | CodOp | — | + | SV40 | + | 45 |
| | | | | | | FXN | | | | | |
| pAAVss | CMV | SV40 | − | − | FXN | CodOp | — | + | SV40 | + | 46 |
| | | | | | | FXN | | | | | |
| pAAVss | CMV | SV40 | − | + | FXN | FXN | — | + | SV40 | + | 47 |
| pAAVss | FXNp | SV40 | − | − | FXN | FXN | — | + | SV40 | + | 48 |
| pAAVss | FXNp | SV40 | − | − | FXN | CodOp | — | + | SV40 | + | 49 |
| | | | | | | FXN | | | | | |
| pAAVss | FXNp | SV40 | − | − | FXN | CodOp | — | + | SV40 | + | 50 |
| | | | | | | FXN | | | | | |
| pAAVss | FXNp | SV40 | − | − | FXN | CodOp | — | + | SV40 | + | 51 |
| | | | | | | FXN | | | | | |
| pAAVss | FXNp | SV40 | − | − | FXN | CodOp | — | + | SV40 | + | 52 |
| | | | | | | FXN | | | | | |
| pAAVss | GFAP | SV40 | − | − | FXN | FXN | — | + | SV40 | + | 53 |
| pAAVss | hSYN | SV40 | − | − | FXN | FXN | — | + | SV40 | + | 54 |
| pAAVss | mCMVe- | SV40 | − | − | FXN | FXN | — | + | SV40 | + | 55 |
| | hEF1p | | | | | | | | | | |
| pAAVss | mCMVe- | SV40 | − | − | FXN | FXN | — | + | SV40 | + | 56 |
| | hEF1p | | | | | | | | | | |
| pAAVss | PGK | SV40 | − | − | FXN | FXN | — | + | SV40 | + | 57 |
| pAAVss | PGK | SV40 | − | − | FXN | CodOp | — | + | SV40 | + | 58 |
| | | | | | | FXN | | | | | |
| pAAVss | PGK | SV40 | − | − | FXN | CodOp | — | + | SV40 | + | 59 |
| | | | | | | FXN | | | | | |
| pAAVss | PGK | SV40 | − | − | FXN | CodOp | — | + | SV40 | + | 60 |
| | | | | | | FXN | | | | | |
| pAAVss | PGK | SV40 | − | − | FXN | CodOp | — | + | SV40 | + | 61 |
| | | | | | | FXN | | | | | |
| pAAVss | SV40 | SV40 | − | − | FXN | FXN | — | + | SV40 | + | 62 |
| pAAVsc | CBA | SV40 | − | − | FXN | FXN | — | + | SV40 | + | 63 |
| pAAVsc | CBA | SV40 | − | − | FXN | FXN | HA(S) | + | SV40 | + | 64 |
| pAAVsc | CBA | SV40 | − | − | FXN | FXN | 3flag | + | SV40 | + | 65 |
| pAAVsc | CBA | SV40 | − | − | FXN | FXN | 3flagcmyc | + | SV40 | + | 66 |
| pAAVsc | CBA | SV40 | − | − | FXN | FXN | cmyc | + | SV40 | + | 67 |
| pAAVsc | CBA | SV40 | − | − | FXN | FXN | cmyc3flag | + | SV40 | + | 68 |
| pAAVsc | CBA | SV40 | − | − | FXN | CodOp | — | + | SV40 | + | 69 |
| | | | | | | FXN | | | | | |
| pAAVsc | CBA | SV40 | − | − | FXN | CodOp | — | + | SV40 | + | 70 |
| | | | | | | FXN | | | | | |
| pAAVsc | CBA | SV40 | − | − | FXN | CodOp | — | + | SV40 | + | 71 |
| | | | | | | FXN | | | | | |
| pAAVsc | CBA | SV40 | − | − | FXN | CodOp | — | + | SV40 | + | 72 |
| | | | | | | FXN | | | | | |
| pAAVsc | CMV | SV40 | − | − | FXN | FXN | — | + | SV40 | + | 73 |
| pAAVsc | CMV | SV40 | − | − | FXN | CodOp | — | + | SV40 | + | 74 |
| | | | | | | FXN | | | | | |
| pAAVsc | CMV | SV40 | − | − | FXN | CodOp | — | + | SV40 | + | 75 |
| | | | | | | FXN | | | | | |
| pAAVsc | CMV | SV40 | − | − | FXN | CodOp | — | + | SV40 | + | 76 |
| | | | | | | FXN | | | | | |
| pAAVsc | CMV | SV40 | − | − | FXN | CodOp | — | + | SV40 | + | 77 |
| | | | | | | FXN | | | | | |
| pAAVsc | CMV | SV40 | − | + | FXN | FXN | — | + | SV40 | + | 78 |
| pAAVsc | FXNp | SV40 | − | − | FXN | FXN | — | + | SV40 | + | 79 |
| pAAVsc | FXNp | SV40 | − | − | FXN | CodOp | — | + | SV40 | + | 80 |
| | | | | | | FXN | | | | | |
| pAAVsc | FXNp | SV40 | − | − | FXN | CodOp | — | + | SV40 | + | 81 |
| | | | | | | FXN | | | | | |
| pAAVsc | FXNp | SV40 | − | − | FXN | CodOp | — | + | SV40 | + | 82 |
| | | | | | | FXN | | | | | |
| pAAVsc | FXNp | SV40 | − | − | FXN | CodOp | — | + | SV40 | + | 83 |
| | | | | | | FXN | | | | | |
| pAAVsc | GFAP | SV40 | − | − | FXN | FXN | — | + | SV40 | + | 84 |
| pAAVsc | PGK | SV40 | − | − | FXN | FXN | — | + | SV40 | + | 85 |
| pAAVsc | PGK | SV40 | − | − | FXN | CodOp | — | + | SV40 | + | 86 |
| | | | | | | FXN | | | | | |
| pAAVsc | PGK | SV40 | − | − | FXN | CodOp | — | + | SV40 | + | 87 |
| | | | | | | FXN | | | | | |
| pAAVsc | PGK | SV40 | − | − | FXN | CodOp | — | + | SV40 | + | 88 |
| | | | | | | FXN | | | | | |
| pAAVsc | PGK | SV40 | − | − | FXN | CodOp | — | + | SV40 | + | 89 |
| | | | | | | FXN | | | | | |
| pAAVss | CB6 | SV40 | − | + | FXN | FXN | — | + | SV40 | + | 90 |
| pAAVsc | CBA | SV40 | − | + | FXN | FXN | — | + | SV40 | + | 91 |
| pAAVss | N/A | SV40 | − | − | FXN | FXN | — | + | SV41 | + | 92 |
| pAAVss | CBA | SV40 | − | − | FXN | FXN | — | + | SV42 | + | 93 |

Plasmid constructs suitable for use in AAV particles include those listed in the sequence listing.

### Example 2. Plasmid Construct Component Modules

Plasmid constructs were designed according to Table 4. The start and stop positions of various components given are relative to the AAV particles described in the sequence listing.

TABLE 4
Component modules or sequence regions of plasmid constructs

| | | SEQ ID NO | | | | | | | |
|---|---|---|---|---|---|---|---|---|---|
| | | 73 | 63 | 79 | 85 | 75 | 70 | 81 | 87 |
| 5′ITR | Start | 65 | 1 | 65 | 65 | 65 | 1 | 65 | 65 |
| | Stop | 169 | 105 | 169 | 169 | 169 | 105 | 169 | 169 |
| Pro- | Start | 205 | 138 | 217 | 214 | 205 | 138 | 217 | 214 |
| moter | Stop | 883 | 789 | 1569 | 735 | 883 | 789 | 1569 | 735 |
| SV40 | Start | 897 | 803 | 1576 | 742 | 897 | 803 | 1576 | 742 |
| | Stop | 1068 | 974 | 1747 | 913 | 1068 | 974 | 1747 | 913 |
| FXN | Start | 1086 | 992 | 1765 | 931 | 1086 | 992 | 1765 | 931 |
| | Stop | 1718 | 1624 | 2397 | 1563 | 1718 | 1624 | 2397 | 1563 |
| Poly(A) | Start | 1798 | 1704 | 2477 | 1643 | 1752 | 1658 | 2431 | 1597 |
| | Stop | 1924 | 1830 | 2603 | 1769 | 1878 | 1784 | 2557 | 1723 |
| 3′ITR | Start | 1968 | 1874 | 2647 | 1813 | 1922 | 1828 | 2601 | 1767 |
| | Stop | 2097 | 2003 | 2776 | 1942 | 2051 | 1957 | 2730 | 1896 |

### Example 3. ELISA Assay for Detecting Differential Payload Expression from Regulatory Elements

The HEK293 cell line was transfected with AAV constructs, SEQ ID Nos. 10-12, 14, 16, 19, 20, 22-26, 29-39, 41-52, 57-61, 63, 70, 73, 75, 79, 81, 85, or 87 to assay the level of expression of a Frataxin payload sequence under control of various regulatory elements. The gWiz-GFP plasmid was co-transfected with each construct as an internal transfection efficiency control. The transfected 293FT cells were harvested 48 hours post-transfection, lysed using the THERMO SCIENTIFIC™ PIERCE™ M-PER™ Mammalian Protein Extraction Reagent, and resuspended in 200 ul of lysis buffer. Protein concentration in each of the samples was measured using the THERMO SCIENTIFIC™ PIERCE™ BCA™ Protein Assay.

ELISA assays for detecting expressed FXN and GFP in cell lysates were performed using the Abcam SimpleStep ELISA kit for FXN and GFP. The results in Tables 5-11 represent Frataxin expression values normalized to GFP. Comparison of expression levels between FXN constructs is represented by further normalization of Frataxin levels relative to reference Frataxin levels.

The results in Tables 5 and 6 represent Frataxin expression values from constructs containing a Kozak Sequence (GCCACCATG) compared to constructs lacking a Kozak sequence.

TABLE 5
Effect of Kozak Sequence on Frataxin Expression In Vitro

| | Relative | SEQ |
|---|---|---|
| Kozak | Expression | ID NO. |
| No | 1.91 | 42 |
| Yes | 1.73 | 47 |
| No | 1.49 | 20 |
| Yes | 0.76 | 30 |
| Yes | 1.24 | 29 |
| No | 0.98 | 12 |

TABLE 6
Effect of Kozak Sequence on Frataxin Expression In Vitro

| | Relative | SEQ |
|---|---|---|
| Kozak | Expression | ID NO. |
| No | 1.45 | 23 |
| Yes | 1.56 | 31 |
| No | 0.76 | 24 |
| Yes | 0.77 | 32 |
| N/A | 1.00 | 12 |

The construct encoding a codon-optimized frataxin driven by the CB6 promoter and lacking a Kozak sequence (SEQ ID NO: 20) gave the highest FXN expression compared to a similar construct with a Kozak sequence (SEQ ID NO: 30).

The results in Table 7 represent Frataxin expression values from constructs comprising a mitochondrial targeting sequence (MTS), also referred to herein as a signal peptide, compared to constructs lacking an MTS. A wild-type FXN MTS (SEQ ID NO: 29) results in the highest level of FXN protein expression in this experiment.

TABLE 7
Effect of Signal Peptide - Mitochondrial Targeting
Sequence on Frataxin Expression In Vitro

| Signal | Relative | Standard | SEQ |
|---|---|---|---|
| Peptide | Expression | Deviation | ID NO. |
| RPL | 0.64 | 0.00 | 37 |
| CS | 0.53 | 0.01 | 26 |
| SOD2 | 0.80 | 0.01 | 39 |
| AH | 0.49 | 0.01 | 25 |
| N/A | 0.98 | 0.02 | 29 |
| N/A | 1.02 | 0.06 | 12 |

The results in Table 8 represent Frataxin expression values from constructs comprising an SV40 intron compared to constructs lacking an SV40 intron. The presence of an SV40 intron had no significant effect on CMV and CB6-driven FXN expression in HEK293 cells.

TABLE 8
Effect of SV40 Intron on Frataxin Expression In Vitro

| SV40 | Relative | Standard | SEQ |
|---|---|---|---|
| Intron | Expression | Deviation | ID NO. |
| Yes | 1.24 | 0.00 | 29 |
| No | 1.03 | 0.02 | 10 |
| Yes | 2.26 | 0.11 | 47 |
| No | 2.43 | 0.23 | 41 |
| No | 1.26 | 0.03 | 11 |
| No | 1.00 | 0.09 | 12 |

The results in Table 9 represent Frataxin expression values from constructs comprising codon optimized frataxin sequences compared to a construct comprising a wild-type frataxin sequence. The effect of Frataxin transgene codon optimization on expression level was variable, with the highest expression levels observed in SEQ ID NOs. 20 and 22.

TABLE 9
Effect of Codon Optimization on Frataxin Expression In Vitro

| Codon | Relative | Standard | SEQ |
|---|---|---|---|
| Optimized | Expression | Deviation | ID NO. |
| Yes | 0.96 | 0.02 | 33 |
| Yes | 0.81 | 0.01 | 34 |
| Yes | 1.32 | 0.10 | 35 |
| Yes | 1.06 | 0.03 | 36 |
| Yes | 1.56 | N/A | 31 |
| Yes | 0.77 | N/A | 32 |
| Yes | 1.72 | 0.04 | 14 |
| Yes | 1.88 | 0.36 | 16 |
| Yes | 1.07 | 0.22 | 19 |
| Yes | 2.21 | 0.20 | 20 |
| Yes | 2.20 | 0.16 | 22 |
| No | 1.00 | N/A | 12 |

The results in Table 10 represent Frataxin expression values from constructs comprising CB6, CMV, or PGK promoter sequences compared to constructs comprising a wild-type frataxin promoter sequence. The values for SEQ ID NO: 12 represent expression levels in two separate experiments. The presence of a CMV or CB6 promoter had the greatest effect of enhancing the expression level of Frataxin approximately 2 fold.

TABLE 10
Effect of CB6, CMV or PGK Promoters
on Frataxin Expression In Vitro

| | Relative | Standard | SEQ |
|---|---|---|---|
| Promoter | Expression | Deviation | ID NO. |
| CB6 | 1.03 | 0.05 | 12 |
| CB6 | 1.00 | 0.09 | 12 |
| CB6 | 1.72 | 0.04 | 14 |
| CB6 | 1.88 | 0.36 | 16 |
| CB6 | 1.07 | 0.22 | 19 |
| CB6 | 2.21 | 0.20 | 20 |
| CB6 | 2.20 | 0.16 | 22 |
| CMV | 1.78 | 0.36 | 43 |
| CMV | 2.16 | 0.17 | 44 |
| CMV | 2.20 | 0.29 | 45 |
| CMV | 2.54 | 0.03 | 46 |
| CMV | 1.63 | 0.12 | 42 |
| PGK | 0.20 | N/A | 57 |
| PGK | 0.23 | N/A | 58 |
| PGK | 0.26 | N/A | 59 |
| PGK | 0.36 | N/A | 60 |
| PGK | 0.72 | N/A | 61 |
| FXN | 0.37 | N/A | 48 |
| FXN | 0.45 | N/A | 49 |
| FXN | 1.07 | N/A | 50 |
| FXN | 1.65 | N/A | 51 |
| FXN | 1.21 | N/A | 52 |

The results in Table 11 represent Frataxin expression values from pAAVsc-Promoter-SV40. The results in Table 11 represent Frataxin expression values from self-complementary AAV constructs comprising CB6, CMV, or PGK promoter sequences compared to constructs comprising a wild-type frataxin promoter sequence. The presence of a CMV promoter had the greatest effect of enhancing the expression level of Frataxin approximately 2-3 fold.

TABLE 11
Effect of CB6, CMV or PGK Promoter on Relative Expression
From pAAVsc-Promoter-SV40 Constructs in 293FT Cells

| Relative | Standard | SEQ |
|---|---|---|
| Expression | Deviation | ID NO. |
| 3.10 | 0.24 | 73 |
| 3.02 | 0.11 | 63 |
| 2.18 | 0.06 | 75 |
| 1.00 | 0.20 | 70 |
| 0.14 | 0.01 | 85 |
| 0.13 | 0.00 | 87 |
| 0.13 | 0.00 | 79 |
| 0.10 | 0.00 | 81 |
| 0.03 | N/A | 73 |

### Example 4. AAV-FXN Constructs and ELISA Assays for Detecting Frataxin Expression

AAV constructs used for this study were designed and built using standard molecular cloning techniques. FXN-tag transgenes were cloned into pAAVsc plasmid and pcDNA3.1 plasmid and the resulting clones were further sequenced to confirm the correctness of all elements such as ITRs, CBA, SV40 and FXN-tags. The pAAVsc-FXN-tag and pcDNA3.1-FXN-tag constructs were transfected to 293FT cells to confirm the expression of FXN-tag proteins. Western blotting using either anti-tag (e.g., anti-HA) or anti-frataxin antibodies confirmed that cloned frataxin-tag is expressed in 293FT cells (data not known).

ELISA assays for detecting expressed FXN-tags were also developed. A three-step sandwich ELISA assay using either anti-tag (e.g. HA, c-myc, flag) or anti-frataxin antibodies as capture antibodies was tested and the ELISA assay may be used for determining frataxin expression levels in CNS delivery studies in vivo. Each serotype was also confirmed by silver-stain-SDS page analysis. The AAV viral genome titers (either self-complementary (sc) or single stranded (ss) AAVs) are described in Table 12.

TABLE 12
AAV Viral Genome titers

| Particles | Genome | LOT NO. | Vg plasmid | Rep-Cap | Ad helper | Titer |
|---|---|---|---|---|---|---|
| AAV2-SEQ | SS | VCAV-01870 | Ald-46819 | VCPV-0136 | Ald-39062A | 0.9 × 10¹³ |
| ID NO: 90 | | | | | | |
| AAV5-SEQ | SS | VCAV-01871 | Ald-46819 | VCPV0128-1 | Ald-39062A | 1.4 × 10¹³ |
| ID NO: 90 | | | | | | |
| AAV6-SEQ | SS | VCAV-01851 | Ald-46819 | VCPV-0132 | Ald-39062A | 0.7 × 10¹³ |
| ID NO: 90 | | | | | | |
| AAV9-SEQ | SS | VCAV-02037 | Ald-48657 | Ald-44533 | Ald-29504 | 1.6 × 10¹³ |
| ID NO: 90 | | | | | | |
| AAVrh10-SEQ | SS | VCAV-01842 | Ald-46819 | VCPV-0135 | Ald-39062A | 1.1 × 10¹³ |
| ID NO: 90 | | | | | | |
| AAVDJ-SEQ | SS | VCAV-01858 | Ald-46819 | VCPI-412 | Ald-39062A | 1.0 × 10¹³ |
| ID NO: 90 | | | | | | |
| AAVDJ/8-SEQ | SS | VCAV-01888 | — | — | — | 1.3 × 10¹³ |
| ID NO: 90 | | | | | | |
| AAVrh10-SEQ | SC | VCAV-01991 | Ald-47191 | VCPV-0135 | Ald-39062A | 0.7 × 10¹³ |
| ID NO: 91 | | | | | | |

### Example 5. In Vivo Frataxin Delivery Study

### Study Design

This study is designed to compare several AAV capsids for payload delivery to the central nervous system (CNS) after direct CNS administration. A total of 160 wild type mice (male, C57BL/6), 10-12 weeks old, were divided into 11 study groups. Mice in each study group were administered an AAV (either self-complementary (sc) or single stranded (ss) AAV) as described in the study design in Table 13. The AAV genomes (either self-complementary (sc) or single stranded (ss) AAVs) with a CB promoter, described in Table 14, were formulated in Phosphate Buffered Saline (PBS), 5% Sorbitol and 0.001% F-68 (Formulation 1) or PBS and 0.001% F-68 (Formulation 2) and administered via intracerebroventricular (ICV) or intrastriatal (IS) injection.

TABLE 13
Mouse Capsid Comparison Study - Design

| Test | | | Total | | | | |
|---|---|---|---|---|---|---|---|
| Articles | AAV | AAV | Dose | | | | End of |
| (Lot No.) | construct | Genome | (VG) | Route | Formulation | n | Study |
| Vehicle | — | — | 0 | ICV | Formulation 1 | 8 | Day 56 |
| | | | | IS | Formulation 1 | 8 | Day 28 |
| VCAV-01801-B | AAV9-SEQ | SC | 5 × 10¹⁰ | ICV | Formulation 1 | 8 | Day 56 |
| | ID NO: 91 | | | IS | Formulation 1 | 8 | Day 28 |
| VCAV-01791 | AAV9-SEQ | SS | 5 × 10¹⁰ | ICV | Formulation 1 | 8 | Day 56 |
| | ID NO: 90 | | | IS | Formulation 1 | 8 | Day 28 |
| VCAV-01870 | AAV2-SEQ | SS | 5 × 10¹⁰ | ICV | Formulation 1 | 8 | Day 56 |
| | ID NO: 90 | | | IS | Formulation 1 | 8 | Day 28 |
| VCAV-01871 | AAV5-SEQ | SS | 5 × 10¹⁰ | ICV | Formulation 1 | 8 | Day 56 |
| | ID NO: 90 | | | IS | Formulation 1 | 8 | Day 28 |
| VCAV-01851 | AAV6-SEQ | SS | 5 × 10¹⁰ | ICV | Formulation 1 | 8 | Day 56 |
| | ID NO: 90 | | | IS | Formulation 1 | 8 | Day 28 |
| VCAV-01842 | AAVrh10-SEQ | SS | 5 × 10¹⁰ | ICV | Formulation 1 | 8 | Day 56 |
| | ID NO: 90 | | | IS | Formulation 1 | 8 | Day 28 |
| VCAV-01858 | AAVDJ-SEQ | SS | 5 × 10¹⁰ | ICV | Formulation 1 | 8 | Day 56 |
| | ID NO: 90 | | | IS | Formulation 1 | 8 | Day 28 |
| VCAV-01888 | AAVDJ/8-SEQ | SS | 5 × 10¹⁰ | ICV | Formulation 1 | 8 | Day 56 |
| | ID NO: 90 | | | IS | Formulation 1 | 8 | Day 28 |
| VCAV-01962-B | AAVrh10-SEQ | SC | 2 × 10⁹ | IS | Formulation 2 | 8 | Day 28 |
| | ID NO: 91 | | | | | | |
| VCAV-01962-T | AAVrh10-SEQ | SC | 5 × 10¹⁰ | IS | Formulation 2 | 8 | Day 28 |
| | ID NO: 91 | | | | | | |

TABLE 14
Mouse Capsid Comparison Study - AAV Genome Sequences

| | | | Genome |
|---|---|---|---|
| | | AAV | SEQ |
| Test article | Capsid | Genome | ID NO |
| VCAV-01801-B | AAV9 | SC | 91 |
| VCAV-01791 | AAV9 | SS | 90 |
| VCAV-01870 | AAV2 | SS | 90 |
| VCAV-01871 | AAV5 | SS | 90 |
| VCAV-01851 | AAV6 | SS | 90 |
| VCAV-01842 | AAVrh10 | SS | 90 |
| VCAV-01858 | AAVDJ | SS | 90 |
| VCAV-01888 | AAVDJ/8 | SS | 90 |
| VCAV-01962-B | AAVrh10 | SC | 91 |
| VCAV-01962-T | AAVrh10 | SC | 91 |

### In Vivo Study

The mice were given a bolus (5×10¹⁰ VG/5 ul total dose at a 1×10¹³VG/mL composition concentration) single unilateral injection (0.5 ul/min) via either intracerebroventricular (ICV) or intrastriatal (IS) administration with a Hamilton 1701 gauge 33 bevel 60 degree needle. The injection site for ICV administration was AP −0.35, ML 1.0, DV −2.5 mm from bregma and the injection site for IS administration was AP 0.5, ML −2.0, DV −4 mm from bregma. Two mm coronal slabs were prepared from the brain. Tissue at the injection site and remaining tissue from the same coronal slab, as well as adjacent slabs immediately anterior (containing frontal cortex) and posterior to the injection site, were taken for human frataxin protein quantitation using the SimpleStep human frataxin ELISA (Abcam) which is specific for human frataxin (no detection of mouse frataxin). Mouse endogenous frataxin protein levels were quantified by a different frataxin ELISA (Abcam).

### Intrastriatal Injection

Human frataxin levels in left striatum (CPuL) on day 28 following IS injection were determined using ELISA assays and the average frataxin expression (ng/mg protein) are shown in Table 15, wherein the AAV genomes are self-complementary (sc) or single stranded (ss). High human frataxin levels were observed following administration of ssAAV9-FXN, ssAAV6-FXN, and ssAAVDJ8-FXN (Lot Nos. VCAV-01791, VCAV-01851, and VCAV-01888). The self-complementary particles, scAAV9-FXN (Lot No. VCAV-01801-B) and scAAVrh10-FXN (Lot No. VCAV-01962-T) resulted in levels 3 and 14 fold higher than single-stranded ssAAV9-FXN (Lot No. VCAV-01791) and ssAAVrh10-FXN (Lot No. VCAV-01842). At the injection site, all the vectors resulted in levels greater than mouse endogenous frataxin, which was measured to be 10 ng/mg protein.

Human frataxin levels in the 2 mm brain slice immediately posterior to the injection site, on day 28 following IS injection were determined using ELISA assays and the average frataxin expression levels (ng/mg protein) are shown in Table 15 (Slab3). ssAAV9, ssAAVDJ8, and ssAAVrh10 (Lot Nos. VCAV-01791, VCAV-01888 and VCAV-01842) IS injected mice had robust expression of human frataxin in the brain, greater than 7-16 fold mouse endogenous frataxin levels, compared to the other viral particles. ssAAV5 (Lot No. VCAV-01871) and ssAAV6 (Lot No. VCAV-01851) IS injected mice resulted in moderate expression, about 2-3 fold mouse frataxin levels, compared to the other viral particles. The results also indicated that ssAAV constructs (e.g., ssAAV 9 (Lot. No. VCAV-01791) and ssAAVrh10 (Lot. No. VCAV-01842)) resulted in higher expression levels than scAAV constructs (e.g., scAAV9 (Lot No. VCAV-01801-B) and scAAVrh10 (Lot No. VCAV-01962-T)).

TABLE 15
Mouse Capsid Comparison Study - Brain Levels
of Human FXN Following IS Injection

| | | Genome | | |
|---|---|---|---|---|
| | AAV | SEQ | CPuL | Slab3 |
| Capsid | Genome | ID NO | AVG ± SEM | AVG ± SEM |
| AAV2 | SS | 90 | 52.10 ± 10.82 | 3.11 ± 0.82 |
| AAV5 | SS | 90 | 75.10 ± 19.56 | 30.80 ± 5.12 |
| AAV6 | SS | 90 | 707.71 ± 203.62 | 16.64 ± 7.06 |
| AAVDJ | SS | 90 | 95.77 ± 29.76 | 6.89 ± 1.04 |
| AAVDJ8 | SS | 90 | 395.14 ± 49.50 | 144.94 ± 18.99 |
| AAV9 | SS | 90 | 809.48 ± 80.21 | 160.89 ± 6.78 |
| AAVrh10 | SS | 90 | 293.58 ± 40.60 | 70.57 ± 16.03 |
| scAAV9 | SC | 91 | 2463.73 ± 357.92 | 4.98 ± 0.47 |
| scAAVrh10-T | SC | 91 | 4233.40 ± 535.40 | 7.71 ± 0.94 |
| scAAVrh10-B | SC | 91 | 176.11 ± 29.10 | 25.89 ± 5.61 |

| | | |
|---|---|---|
| Sorbitol formulation (background) | 0.02 ± 0.01 | 0.02 ± 0.01 |

### ICV Injection

On day 56 following ICV injection, human frataxin protein levels were measured by ELISA at the injection site (Ing. Site, Table 16) and surrounding tissue from the same 2 mm slice (Slab 2, Table 16). In addition, human frataxin protein levels were measured by ELISA in a 2 mm slice immediately anterior (containing mostly frontal cortex, Slab 1, Table 16), and in a 2 mm slice immediately posterior (Slab 3, Table 16). The AAV genomes were self-complementary (sc) or single stranded (ss). The injection of ssAAV6-FXN VCAV-01851 resulted in moderate expression at the injection site (5 fold mouse frataxin). The self-complementary vector scAAV9-FXN VCAV-01801-B resulted in high expression levels, 100 fold higher than single-stranded ssAAV9-FXN VCAV-01791. The spread of human frataxin expression following ssAAV2-FXN VCAV-01870 and ssAAV5-FXN VCAV-01871 is limited, with highest levels found at the injection site and much lower levels anterior (Slab 2) and posterior (Slab 3) to the injection site. In contrast, ssAAV9-FXN VCAV01791 and ssAAVrh10-FXN VCAV-01842 show a more consistent expression levels in the different antero-posterior samples analyzed. ssAAVDJ-FXN VCAV-1858 and ssAAVDJ8-FXN VCAV-01888 resulted in similar levels of human frataxin. Of the single-stranded vectors, ssAAV6-FXN VCAV-01851 resulted in the highest maximum and overall levels of FXN expression.

TABLE 16
Mouse Capsid Comparison Study - Brain Levels of human FXN Following ICV Injection

| | | Genome | | | | |
|---|---|---|---|---|---|---|
| | AAV | SEQ | Slab1 | Slab2 | Inj. Site | Slab3 |
| Capsid | Genome | ID NO | AVG ± SEM | AVG ± SEM | AVG ± SEM | AVG ± SEM |
| AAV2 | SS | 90 | 0.07 ± 0.01 | 0.28 ± 0.04 | 1.98 ± 0.59 | 0.18 ± 0.06 |
| AAV5 | SS | 90 | 0.13 ± 0.06 | 1.68 ± 0.89 | 47.53 ± 15.53 | 5.41 ± 1.33 |
| AAV6 | SS | 90 | 1.06 ± 0.14 | 12.52 ± 2.40 | 53.23 ± 12.28 | 23.11 ± 4.93 |
| AAVDJ | SS | 90 | 1.82 ± 0.25 | 2.80 ± 0.60 | 9.27 ± 3.32 | 0.97 ± 0.22 |
| AAVDJ8 | SS | 90 | 0.92 ± 0.34 | 0.99 ± 0.48 | 3.94 ± 2.56 | 0.31 ± 0.07 |
| AAV9 | SS | 90 | 0.60 ± 0.08 | 1.28 ± 0.61 | 1.99 ± 0.90 | 1.36 ± 1.07 |
| AAVrh10 | SS | 90 | 0.35 ± 0.05 | 0.40 ± 0.04 | 0.35 ± 0.03 | 0.17 ± 0.02 |
| AAV9 | SC | 91 | 5.52 ± 1.30 | 39.48 ± 12.40 | 197.15 ± 66.93 | 4.64 ± 1.17 |

| | | | | |
|---|---|---|---|---|
| Sorbitol formulation (background) | 0.04 ± 0.01 | 0.05 ± 0.03 | 0.06 ± 0.02 | 0.04 ± 0.02 |

### Example 6. In Vivo ICV (Lateral Ventricle) and CM (Cisterna Magna) Injection Study

To compare expression following intracerebroventrical (ICV, lateral ventricle) or intracisternal (Cisterna Magna, CM) injections, more than 18 wild type mice (C57BL/6), 7 weeks old, were administered an AAV as described in the study design in Table 17. The AAV, described in Table 18, was formulated in PBS and 0.001% F-68 and administered via intracerebroventricular (ICV, lateral ventricle) or intracisternal (CM, cisterna magna) injection.

TABLE 17
Mouse ICV vs CM Study - Design

| AAV | Dose | | | End of |
|---|---|---|---|---|
| Genome | (VG) | Route | n | Study |
| SS | 5 × 10¹⁰ | ICV | 6 | Day 28 |
| | | CM | 6 | Day 28 |
| N/A | 0 | ICV | 3 | Day 28 |
| | | CM | 3 | Day 28 |

TABLE 18
Mouse ICV vs CM - AAV Construct Sequences

| | | Genome |
|---|---|---|
| AAV | AAV | SEQ |
| Capsid | Genome | ID NO |
| rh10 | SS | 90 |

The mice were given a bolus (5×10¹⁰ VG/5 ul total dose at a 1×10¹³ VG/mL composition concentration) single unilateral injection (0.5 ul/min) via either intracerebroventricular (ICV) or intracisternal (CM) administration. 22 samples were taken for each administration group after 28 days.

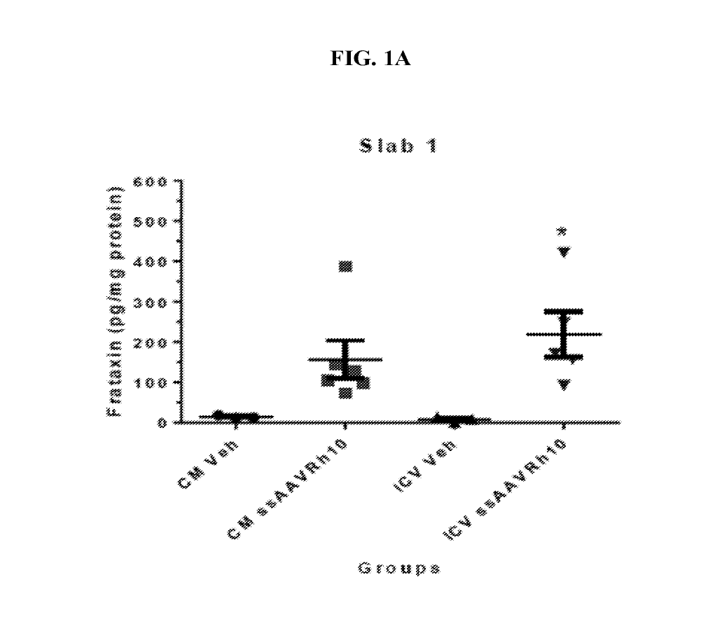

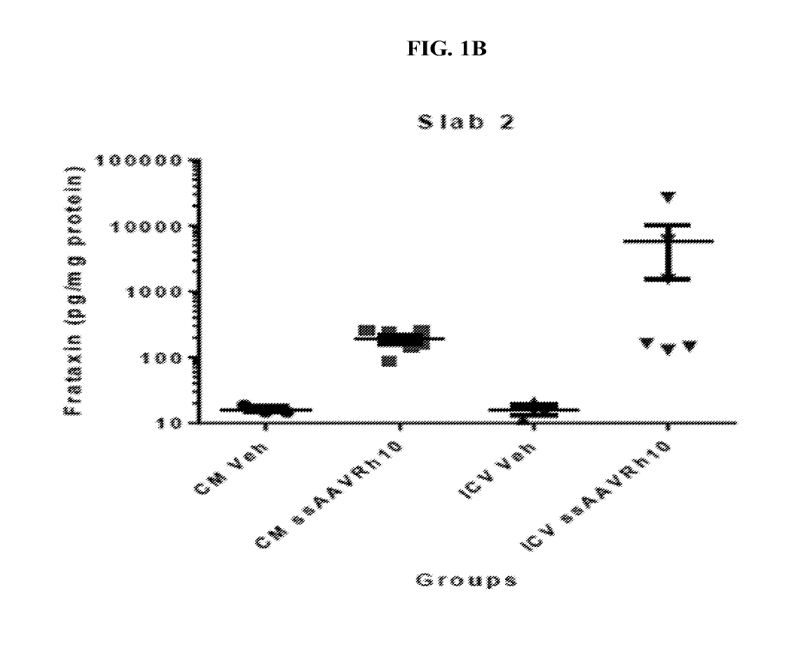

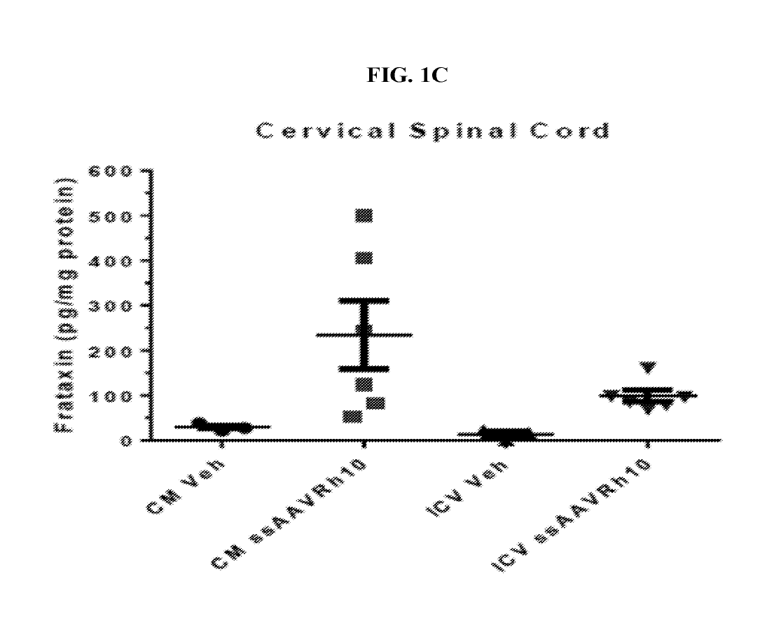

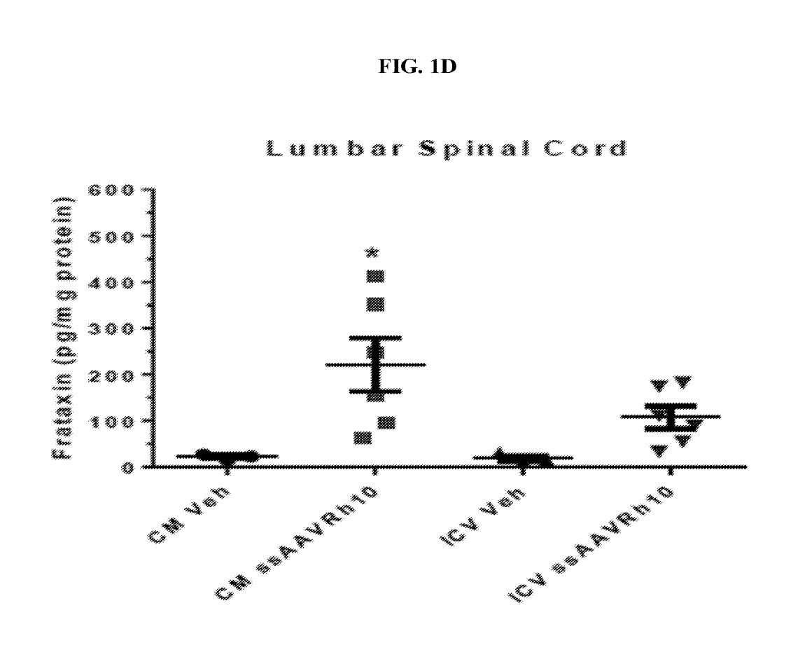

On day 28, human frataxin levels in a 2 mm anterior slice containing mostly frontal cortex (Slab 1), an adjacent 2 mm slice containing striatum (Slab 2), cervical spinal cord and lumbar spinal cord were determined using the SimpleStep human frataxin ELISA assay (Abcam) specific for human frataxin protein, and the average frataxin expression levels (ng/mg protein) are shown in FIG. 1A-1D. Frataxin protein expression was higher in brain slices after ICV administration than after CM administration (FIG. 1A-1B) whereas frataxin protein expression was higher in cervical and lumbar spinal cord after CM administration than after ICV administration (FIG. 1C-1D).

### Example 7. Non-Human Primate Capsid Comparison Study of AAV Delivery Via Intrathecal Lumber (IT-L) Administration

### Animal Test System

Forty male cynomolgus monkeys (Covance, Inc., Alice, Tex.) approximately 3 to 5 years old (approximately 3 to 5 kg) were used for this study. All the animals were bred for this study only and received no other experimental treatments. The test animals were screened for neutralizing antibody titers to the viral particles that they would later be administered, to confirm seronegativity before inclusion into this study. All the screening results were recorded for each animal and each animal had an AAV antibody titer less than 1:10.

All the test animals were first quarantined for at least four weeks at the study center with daily observations.

All the animals were housed in standard animal cages in compliance with the Guide for the Care and Use of Laboratory Animals, DHHS, (NIH) No. 86-23, and the Animal Welfare Act (9 CFR 3). The animals were also provided with environmental enrichment as per AAALAC (Association for Assessment and Accreditation of Laboratory Animal Care) accredited program.

### Test Articles/Particles

Eight different AAV test articles (Test particles) encoding human frataxin (FXN) were delivered via intrathecal lumber (IT-L) implanted catheter. AAV2, AAV5, AAV6, AAV9, AAVrh10, AAVDJ and AAVDJ8 serotypes which were packaged with a transgene (FXN) under the CB6 promoter were tested and compared for distribution and level of transgene (FXN) expression in NHP spinal cord and dorsal root ganglia (DRGs). The test particles used in this study are listed in Table 19, wherein the AAV genomes are self-complementary (SC) or single stranded (SS).

TABLE 19
NHP Capsid Comparison Study - Test Articles

| | | | | Genome |
|---|---|---|---|---|
| Test | | AAV | | SEQ |
| articles | Lot No. | Capsid | Genome | ID NO. |
| 1 | VCAV-01870 | AAV2 | SS | 90 |
| 2 | VCAV-01871 | AAV5 | SS | 90 |
| 3 | VCAV-01851 | AAV6 | SS | 90 |
| 4 | VCAV-02037 | AAV9 | SS | 90 |
| 5 | VCAV-01842 | AAVrh10 | SS | 90 |
| 6 | VCAV-01858 | AAVDJ | SS | 90 |
| 7 | VCAV-01888 | AAVDJ/8 | SS | 90 |
| 8 | VCAV-01991 | AAVrh10 | SC | 91 |

The test articles comprised preparations of vector with and without sorbitol. The test articles and AAV vehicle (PBS with 0.001% F-68) were stored at the dosing concentration of 1×10¹³ vg/mL at −75° C. or below; and were thawed at room temperature on the day of administration.

### Surgery and Surgical Recovery

38 cynomolgus monkeys (including 4 alternates) were surgically implanted with IT-L catheters following the procedures as described below.

For IT-L catheter implantation, an incision was made over the dorsal process of the lumbar spine at L4, L5, or L6. The muscle was dissected and a hemilaminectomy was made for the insertion of a polyethylene lined polyurethane open ended catheter. The catheter was then advanced to the area of the thoraco-lumbar junction. The proximal end of the catheter terminates in a low volume titanium subcutaneous access port (MIN LOVOL, Access Technologies). Proper IT-L sampling catheter placement was verified by the ability to collect CSF at surgery. The skin was closed with sutures and tissue adhesive.

Catheter patency was verified by myelogram with Isovue 300. Dosing was performed 7-14 days after surgery.

### Experimental Design

Cynomolgus monkeys (Male 3-5 years of age with an AAV antibody titer of <1:10; including 4 alternates) surgically implanted with IT catheters in the lumbar region (L1) were divided into 9 study groups and each animal was administered three 1.0 ml slow bolus injections of test article dosing solution delivered via IT catheter at 60 minute intervals (i.e. 0 minute, 60 minutes and 120 minutes on day 1 of the study) followed by 0.2 ml of AAV vehicle immediately after the third injection to flush the dose from the catheter system. Study groups are shown in the following Table 20, wherein the AAV genomes are self-complementary (SC) or single stranded (SS).

TABLE 20
NHP Capsid Comparison Study - Study Design

| | No. | | | | | Day of |
|---|---|---|---|---|---|---|
| | of an- | Test | Ge- | | Conc. | Nec- |
| Group | imals | articles | nome | Dose (vg) | (vg/ml) | ropsy |
| 1 | 2 | AAV2-SEQ | SS | 3 × 10¹³ | 1 × 10¹³ | 43 |
| | | ID NO: 90 | | | | |
| 2 | 4 | AAV5-SEQ | SS | 3 × 10¹³ | 1 × 10¹³ | 43 |
| | | ID NO: 90 | | | | |
| 3 | 4 | AAV6-SEQ | SS | 3 × 10¹³ | 1 × 10¹³ | 43 |
| | | ID NO: 90 | | | | |
| 4 | 4 | AAV9-SEQ | SS | 3 × 10¹³ | 1 × 10¹³ | 43 |
| | | ID NO: 90 | | | | |
| 5 | 4 | AAVrh10-SEQ | SS | 3 × 10¹³ | 1 × 10¹³ | 43 |
| | | ID NO: 90 | | | | |
| 6 | 4 | AAVDJ-SEQ | SS | 3 × 10¹³ | 1 × 10¹³ | 43 |
| | | ID NO: 90 | | | | |
| 7 | 4 | AAVDJ/8-SEQ | SS | 3 × 10¹³ | 1 × 10¹³ | 43 |
| | | ID NO: 90 | | | | |
| 8 | 4 | AAVrh10-SEQ | SC | 3 × 10¹³ | 1 × 10¹³ | 43 |
| | | ID NO: 91 | | | | |
| 9 | 4 | Vehicle | | N/A | N/A | 43 |

During the period of the study, in-life observations and measurements such as body weights, food consumption, clinical observations, and clinical pathology evaluations, and other observations were recorded.

No significant adverse effects were observed during the study as reported in these in-life parameters of each animal.

On Day 43, the animals were sacrificed and selected tissues harvested for bioanalytical evaluation.

### Example 8. ALT and AST Analysis of Tested Animals

Serum samples from animals treated with AAV particles that were collected on Day 1 (immediately prior to dosing), and Day 43 (just prior to necropsy) were analyzed for Alanine Aminotransferase (ALT) and Aspartate Aminotransferase (AST) levels.

In cynomolgus monkeys, the median values and reference intervals for ALT were 33 U/L and 15-64 U/L; respectively; and the median values and reference intervals for AST were 31 U/L and 20-63 U/L.

For most test articles, ALT and AST serum levels were not elevated. The AAVrh10-treated animals showed an increase in ALT and AST levels. Additional results from another NHP pilot study also indicated the scAAVrh10-FXN can induce elevation of ALT and AST in the blood. As a group, scAAVrh10 ALT values were 3-folder higher than the upper reference interval.

### Example 9. Transgene (Frataxin) mRNA Expression Analysis

Frataxin mRNA expression in all the tissues collected at necropsy as described in Example 8 was measured using q-PCR. All samples were run in duplex qRT-PCR reactions for frataxin and control mRNAs (beta-actin and GAPDH). A statistical test (Grubbs test) was used to determine single outliers values in each data set. The fold-change ratios were used to determine single outliers among replicates. For all the samples tested, rates of exclusion for replications were 5.9% of 816 spinal cord values, 4.6% of 544 DRG values, and 1.6% of 272 dentate nucleus and liver values. The absolute and relative quantification for frataxin mRNA expression by qRT-PCR were also determined.

Overall, the 8 AAV serotypes injected intrathecally led to human frataxin expression levels higher than 0.5 fold over the human brain reference control. After 6 weeks, all tested animals showed a rise in neutralizing AAV antibodies of the injected serotype in serum and in CSF.

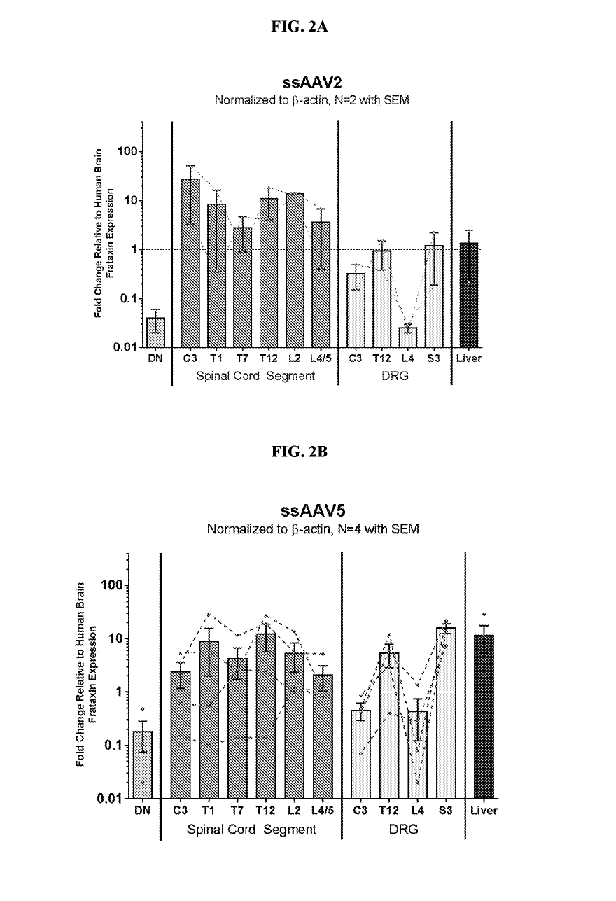

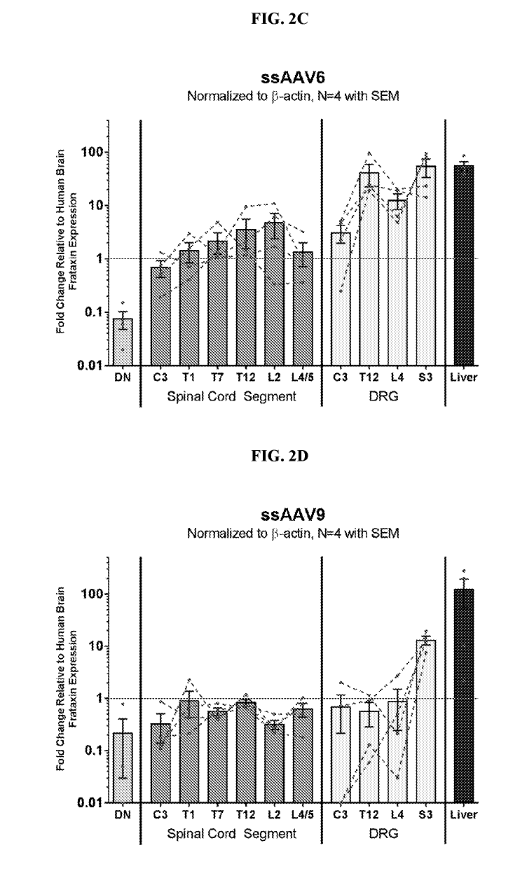

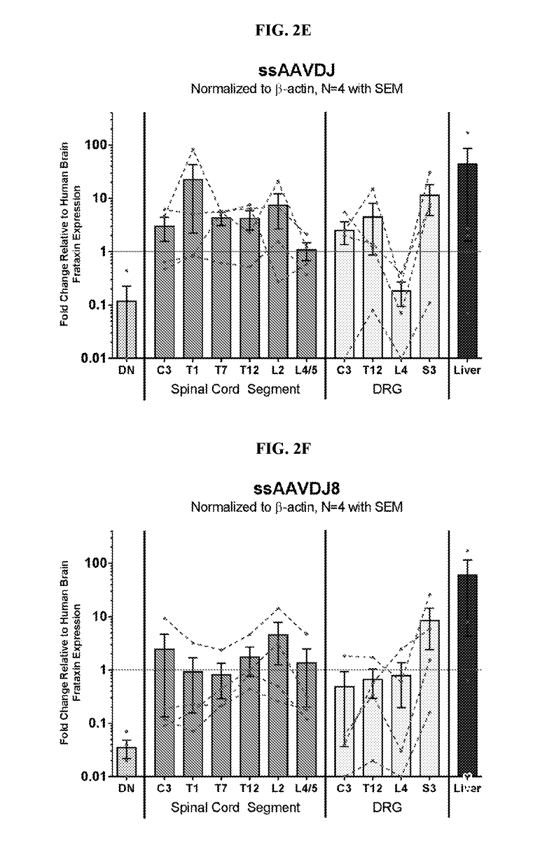

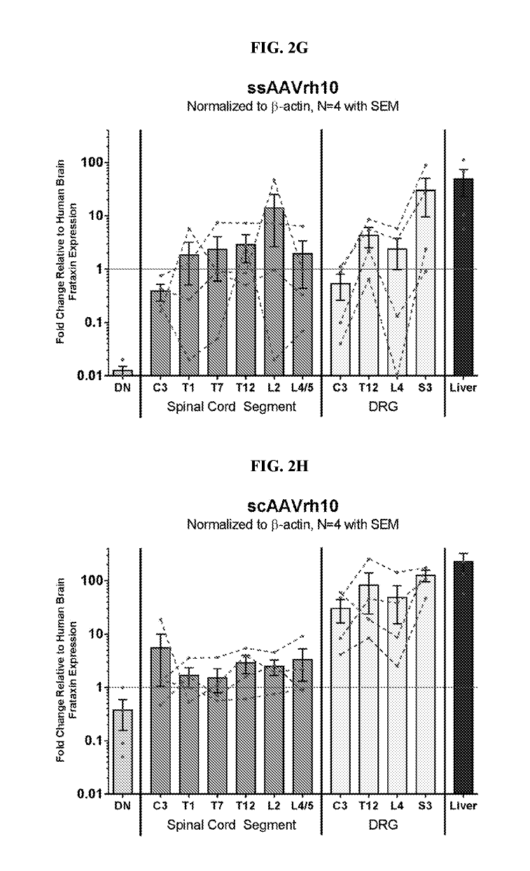

Frataxin mRNA expression levels in the CNS and selected peripheral organs in tested monkeys after IT-L1 bolus dosing of each capsid are shown in FIG. 2A to 2H.

ssAAV2 serotype: Frataxin mRNA expression is low in dentate nucleus at 4% human brain level. The expression in spinal cord is 2.8-27.2 fold over human brain level, but no apparent rostro-caudal gradient was observed in the study. AAV2-FXN expression is variable in DRGs with about 0.025 to 1.2 fold over human brain level. In general, the AAV2-FXN expression in DRGs is lower than that in spinal cord. Similarly, the average expression level of AAV2-FXN in liver is the lowest of all AAV capsids tested, normalized to human brain levels (FIG. 2A). As compared to the other serotypes, AAV2 generates the highest average expression level of frataxin in spinal cord (n=2).

ssAAV5 serotype: Frataxin mRNA expression is third highest in dentate nucleus at 18% human brain level. The expression in spinal cord is 2.1-12.3 fold over human brain level, but no apparent rostro-caudal gradient was observed in the study. AAV5-FXN expression is variable in DRGs with about 0.43 to 15.9 fold over human brain level. The AAV5-FXN expression in liver roughly matches peak levels in the spinal cord and DRGs (FIG. 2B).

ssAAV6 serotype: Frataxin mRNA expression is low in dentate nucleus at 7% human brain level. The expression in spinal cord is 0.69-4.8 fold over human brain level, and a rostro-caudal gradient from C3 to L2 in expression was observed in the study, with levels highest at L2. The highest frataxin mRNA expression level in DRGs was detected for AAV6-FXN compared with all ssAAV treated groups. The AAV6-FXN expression in DRGs ranges from 3.1 to 54.1 fold over human brain level. DRGs show greater AAV6-FXN expression levels than in matching spinal cord segments C3, T12 and L4. Furthermore, the observed expression pattern in DRGs is similar with the matching spinal cord segments. The average expression level of AAV6-FXN in liver is high, 55.9 fold of human brain level, similar to the high levels of frataxin expression in T12 and S3 DRGs (FIG. 2C).

ssAAV9 serotype: The highest frataxin mRNA expression level in dentate nucleus was detected for ssAAV9 serotype compared with other ssAAV treated groups at 22% human brain level. The frataxin expression in spinal cord ranges from 0.32 to 0.9 fold over human brain level, and is generally consistent across spinal cord segments. The average ssAAV9-FXN expression in DRGS is similar to that in the spinal cord, but an elevated expression in S3 DRGs (n=4 animals) was detected at 13.1 fold over human brain levels. The expression pattern in DRGs is more variable than that in the spinal cord. ssAAV9 leads to the highest expression in liver of all ssAAV treated groups at 123 fold over human brain level, which exceeds all other samples in the study (FIG. 2D).

ssAAVDJ serotype: Frataxin mRNA expression in dentate nucleus is at 12% human brain level. After ssAAVDJ-FXN administration, frataxin expression peaked at T1 segments in the spinal cord with a range from 1.1 to 22.6 fold over human brain level. The expression in C3 and T12 DRGs is similar to that in the spinal cord, but the expression in L4 DRGs is about 5 fold lower than that in L4/L5 segments in the spinal cord. ssAAVDJ-FXN is expressed in liver higher than all the CNS tissues tested. The expression in liver is about 44.1 fold over human brain level (FIG. 2E).

ssAAVDJ8 serotype: Frataxin mRNA expression in dentate nucleus is low at 3% human brain level. The ssAAVDJ8-FXN expression is consistent across the spinal cord ranging from 0.93-4.6 fold over human brain level. The average expression in DRGs is consistent with the matching spinal cord segments, with a range from 0.49-0.78 in segments from C3 to L4 and 8.4 fold in S3. Similar to the ssAAVDJ serotype, ssAAVDJ8 has relatively high expression level in liver compared with other ssAAV treated groups. The expression in liver is about 60.2 fold over human brain level (FIG. 2F).

ssAAVrh10 serotype: Frataxin mRNA expression in dentate nucleus is very low. The ssAAVrh10-FXN expression in the spinal cord ranges from 0.39-14 fold over human brain level, with rostro-caudal gradient of expression across C3 to L2, with levels highest at L2. The average expression in DRGs is similar to the matching spinal cord segments and elevated in S3 segment to 30.2 fold over human brain level. This result is similar to that in ssAAVDJ8 and ssAAV9 driven transgene expression, indicating these three serotypes ssAAVrh10, ssAAVDJ8 and ssAAV9 have high transduction efficiency to the S3 segment of the spinal cord and matching DRGs. The expression in liver is about 49 fold over human brain level, slightly greater than the highest DRG level (FIG. 2G).

scAAVrh10 serotype: Frataxin mRNA expression in dentate nucleus is the highest among the tested capsid at 37% human brain level. The scAAVrh10-FXN expression is consistent across the spinal cord from C3 to L4/5, ranging from 1.5-5.6 fold over human brain level. The expression pattern across the spinal cord is more homogeneous than that of ssAAVrh10 serotype. scAAVrh10 can drive a similar level of expression as compared to the ssAAV treated groups but the level is lower than that of ssAAV2. The frataxin mRNA expression in DRGs is consistently high at all segments ranging from 30.4-127 fold over human brain level. Similarly, the expression in liver is the highest among all the tested groups at about 229 fold over human brain level, with a highest individual liver expression of 500 fold over human brain level (FIG. 2H).

### Example 10. Comparison of Transgene (Frataxin) mRNA Expression in NHPs Treated with Different Serotypes

Frataxin mRNA expression levels obtained from different tissue samples from all treated groups were further compared.

The expression levels from ssAAVrh10 and scAAVrh10 were compared. The results indicate that Frataxin expression is greatly improved in scAAVrh10 as compared to ssAAVrh10, while both viral particles show a similar average range of expression throughout the spinal cord. DRG expression in the scAAVrh10 treated animals is driven above the matching spinal cord segment expression and is more uniform across the spinal cord. There is also about 4.5 fold difference in average liver expression between scAAVrh10 and ssAAVrh10 (FIGS. 2G and 2H).

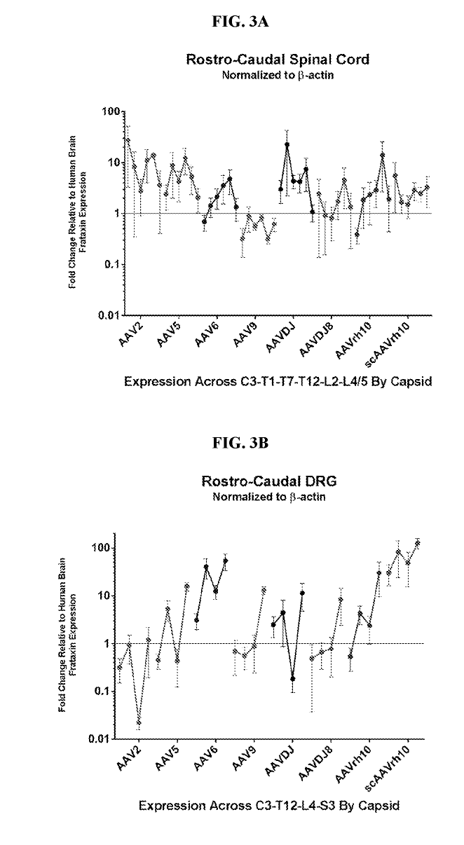

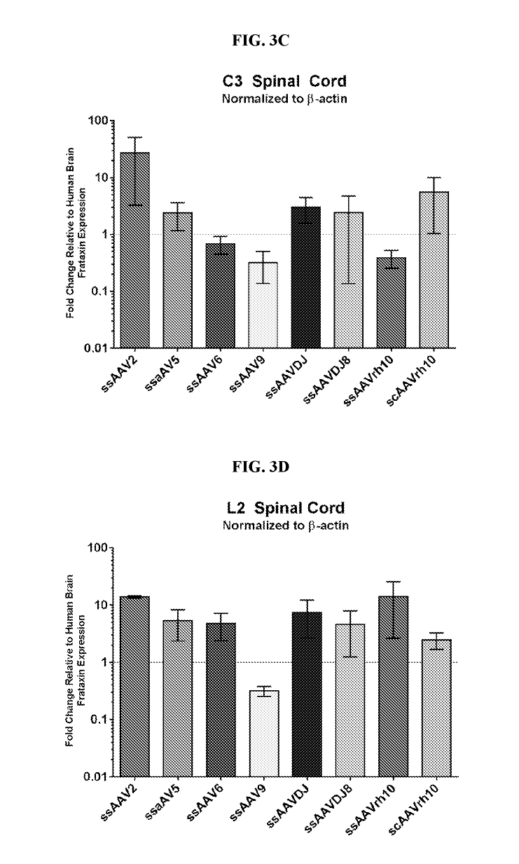

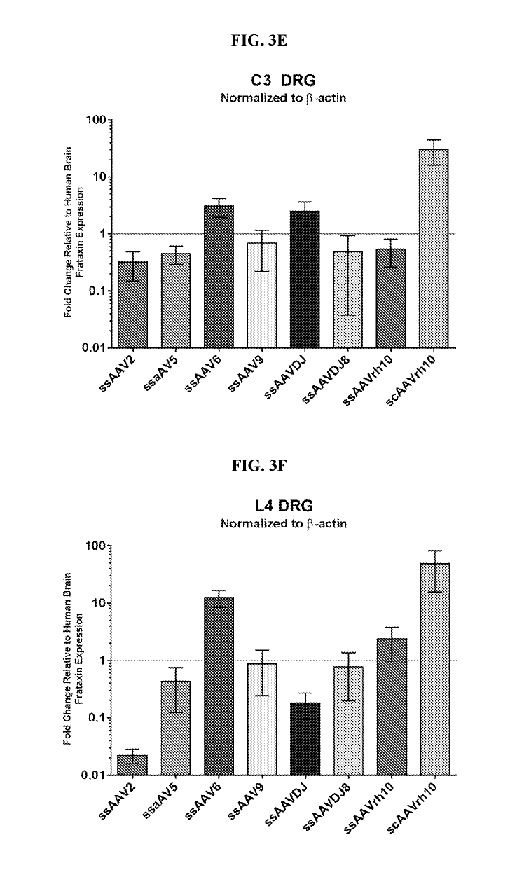

Frataxin expression profiles for each capsid serotypes in spinal cord segments and matching DRGs also show that certain serotypes result in much higher transgene expression than others. As shown in FIG. 3A, capsids, in order of highest average frataxin mRNA expression across the tested spinal cord segments, were ranked: ssAAV2 (11×)>ssAAVDJ (7×)>ssAAV5 (6×)>ssAAVrh10 (4×)>scAAVrh10 (3×)>ssAAV6 (2×)>ssAAVDJ8 (2×)>ssAAV9 (0.6×). As shown in FIG. 3B, capsids, in order of highest average frataxin mRNA expression across the tested dorsal root ganglia (DRG), were ranked: scAAVrh10 (72×)>ssAAV6 (28×)>ssAAVrh10 (9×)>ssAAV5 (6×)>ssAAVDJ (5×)>ssAAV9 (4×)>ssAAVDJ8 (3×)>ssAAV2 (0.6×).

A comparison of frataxin expression by each capsid along the rostro-caudal axis of the spinal cord reveals that most of capsids tested have similar ranges of expression in the L2 segment, one segment caudal to the L1 site for administration, with the exception of ssAAV9 which results in less than 1/10th of the expression with other capsids (FIG. 3D). The C3 segment, the most rostral segment tested, demonstrates pronounced differences in expression across capsids tested and increased animal to animal viability for several AAV serotypes, notably ssAAVDJ8 and ssAAV2 (FIG. 3C). Furthermore, the expression in C3 segment is reduced in ssAAV6 and ssAAVrh10 treated animals, and slightly reduced (or consistent) in ssAAV5, ssAAVDJ and scAAVrh10 treated animals, as compared to the expression in L2 segment. Among the tested capsids, ssAV2 has the highest overall frataxin mRNA level across all tested spinal cord segments (FIG. 3C and FIG. 3D).

A similar comparison of frataxin expression by capsids in C3 and L4 DRGs reveals that among tested DRGs, C3 and L4 show lower levels of expression in all tested capsids, as compared to the expression levels in T12 and S3 DRGs. The results also indicate that only ssAAV6 and scAAVrh10 treatments result in frataxin mRNA expression above human brain level in both tissues. Consistent levels of expression were detected in ssAAV5 and ssAAV9 DRGs, but a more variable pattern was detected in ssAAVDJ8 DRGs. In ssAAV2 and ssAAVDJ treatments, frataxin mRNA expression is reduced significantly in L4 DRGs as compared to C3 DRGs. In addition, both ssAAVrh10 and scAAVrh10 show a slight increase in frataxin expression in L4 DRGs as compared to C3 DRGs. ssAAV2 is the strongest overall performer in overall spinal cord expression, but the poorest in overall DRG expression (FIG. 3E and FIG. 3F).

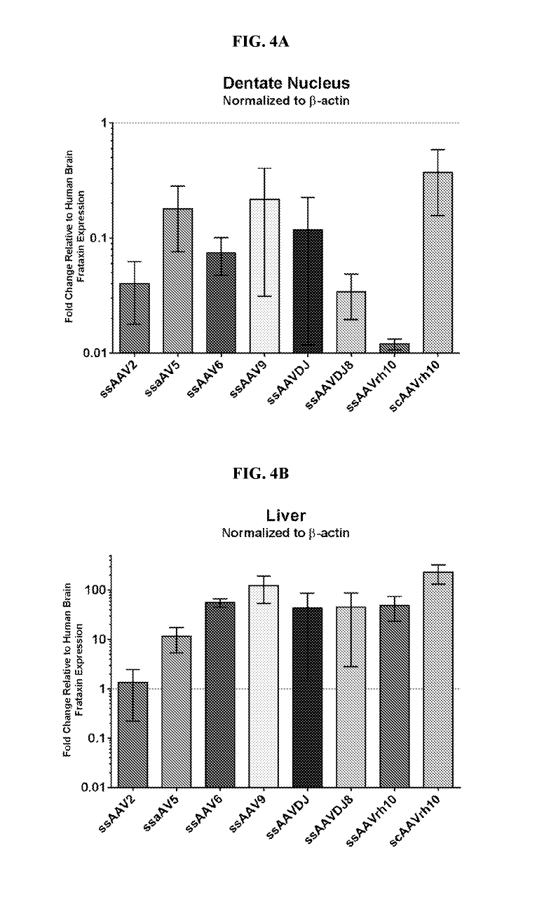

Frataxin mRNA expression in dentate nucleus reflects the ability of a capsid to penetrate into the deeper brain region after intrathecal injection. The results suggest that the expression in dentate nucleus was achieved in the scAAVrh10, ssAAV9 and ssAAV5 treatment groups. ssAAVrh10 consistently resulted in the lowest levels of frataxin expression in the dentate nucleus (FIG. 4A).

Frataxin mRNA expression in liver for all tested capsids was evaluated to determine the extent of extra-CNS transduction. In most animals, frataxin expression in liver is greater than that in human brain (FIG. 4B).

### Example 11. ELISA Assay for Detecting Differential Payload Expression from Regulatory Elements in Various Cell Types

The HEK293 cell line was transfected with AAV constructs, SEQ ID Nos. 13-22, 40, 48, 54-56, 62, 63, 70, 73, 75, 79, 81, 85, 87, 92, and 93 to assay the level of expression of a Frataxin payload sequence under control of various regulatory elements in human embryonic kidney 293 (HEK 293), primary human fibroblast (FA), rat primary dorsal root ganglia (DRG) neurons (rDRG), or human induced pluripotent stem cell (iPSC) derived neural stem cells (hNSC) cell types.

HEK 293 cells were co-transfected in triplicate using FUGENE® HD reagent with each construct (0.5 μg) and the gWiz-GFP plasmid (100 ng) as an internal transfection efficiency control. The transfected 293FT cells were harvested 30-36 hours post-transfection, lysed using the THERMO SCIENTIFIC™ PIERCE™ M-PER™ Mammalian Protein Extraction Reagent, and resuspended in 200 ul of lysis buffer. Protein concentration in each of the samples was measured using the Thermo Scientific™ Pierce™ BCA™ Protein Assay.

Primary human fibroblast, HIP™ Neural Stem Cells (Human iPSC-Derived), and rat primary dorsal root ganglia (DRG) neurons (rDRG) cells were co-transfected in triplicate using Lipofectamine LTX-Plus reagent with each construct (0.5 μg) and the gWiz-GFP plasmid (100 ng) as an internal transfection efficiency control. The transfected 293FT cells were harvested 30-36 hours post-transfection, lysed using the THERMO SCIENTIFIC™ PIERCE™ M-PER™ Mammalian Protein Extraction Reagent, and resuspended in 200 ul of lysis buffer. Protein concentration in each of the samples was measured using the THERMO SCIENTIFIC™ PIERCE™ BCA™ Protein Assay.

ELISA assays for detecting expressed FXN and GFP in cell lysates were performed using the Abcam SimpleStep ELISA kit for FXN and GFP. The results in Tables 21-23 represent exogenous Frataxin expression, subtracting endogenous Frataxin, values relative to GFP. Comparison of expression levels between FXN constructs is represented by normalized Frataxin compared to reference Frataxin levels.

The results in Table 21 represent Frataxin expression values from constructs comprising hEF1α, hEF1α (cpg free), CB6, frataxin (FXN), human synapsin (hSYN), SV40, ITR or CBA promoter sequences. Expression in the ITR construct is driven only by the AAV ITR sequence without any additional promoter sequence. The constructs were expressed in human embryonic kidney 293 (HEK 293), primary human fibroblast (FA), rat primary dorsal root ganglia (DRG) neurons (rDRG), or human induced pluripotent stem cell (iPSC) derived neural stem cells (hNSC). Constructs expressing Frataxin driven by a hEF1a promoter results in highest FXN expression in primary fibroblast cells. The presence of a CpG island in the hEF1a promoter promotes FXN expression in all cell types tested compared to CpG-free hEF1a promoter.

TABLE 21
Effect of Promoter on Driving Frataxin Expression
in Various Cell Types In Vitro

| | | | | | SEQ |
|---|---|---|---|---|---|
| Promoter | HEK293 | FA | rDRG | hNSC | ID NO. |
| hEF1α (CpG free) | 0.12 | 6.36 | 0.89 | 0.37 | 55 |
| hEF1α | 1.33 | 8.67 | 1.26 | 1.12 | 56 |
| CB6 | 3.09 | 1.46 | 1.19 | 1.52 | 40 |
| FXN | 0.06 | 0.00 | 0.02 | 0.14 | 48 |
| hSYN | 0.12 | 0.03 | 0.15 | 0.16 | 54 |
| ITR | 0.03 | 0.04 | 0.02 | | 92 |
| SV40 | 0.16 | 0.44 | 0.30 | 0.33 | 62 |
| CBA | 1.00 | 1.00 | 1.00 | 1.00 | 93 |

The results in Table 22 represent Frataxin expression values from self-complementary AAV (scAAV) constructs comprising frataxin (FXN), PGK, CBA, or CMV promoter sequences expressed in primary human fibroblast (FA) cells.

The presence of a CMV promoter had the greatest effect of enhancing the expression level of Frataxin in primary fibroblast cells.

TABLE 22
Effect of Promoter on Driving Frataxin Expression
in Primary Human Fibroblast Cells In Vitro

| Frataxin | | Relative | SEQ |
|---|---|---|---|
| Sequence | Promoter | Expression | ID NO. |
| FXN | FXN | 0.05 | 79 |
| FXN | PGK | 0.05 | 85 |
| FXN | CBA | 0.14 | 63 |
| FXN | CMV | 0.49 | 73 |
| Codon Optimized FXN | FXN | 0.06 | 81 |
| Codon Optimized FXN | PGK | 0.03 | 87 |
| Codon Optimized FXN | CBA | 0.44 | 70 |
| Codon Optimized FXN | CMV | 1.06 | 75 |

The results in Table 23 represent Frataxin expression values from constructs comprising codon optimized frataxin sequences compared to a construct comprising a wild-type frataxin sequence. The constructs were expressed in human embryonic kidney 293 (HEK 293), primary human fibroblast (FA), rat primary dorsal root ganglia (DRG) neurons (rDRG), or human induced pluripotent stem cell (iPSC) derived neural stem cell (hNSC) cell types. The constructs encoding a codon-optimized FXN were observed to have higher levels of expression than native FXN in all cell types tested.

TABLE 23
Effect of Codon Optimization on Relative Frataxin Expression
from pAAVsc-Promoter-SV40 Constructs in Various Cell Types

| Relative | | | | | SEQ |
|---|---|---|---|---|---|
| Expression | HEK293 | hFibroblast | rDRG | hNSC | ID NO. |
| Opti-1 | 2.51 | 2.37 | 1.72 | 1.67 | 13 |
| Opti-2 | 3.30 | 2.58 | 2.65 | 2.71 | 14 |
| Opti-3 | 3.24 | 1.34 | 1.55 | N/A | 15 |
| Opti-4 | 2.67 | 1.64 | 2.02 | 1.20 | 16 |
| Opti-5 | 3.04 | 1.69 | 1.96 | N/A | 17 |
| Opti-6 | 3.63 | 1.65 | 1.70 | N/A | 18 |
| Opti-7 | 3.25 | 2.15 | 2.12 | 2.43 | 19 |
| Opti-8 | 2.60 | 1.93 | 2.42 | 3.99 | 20 |
| Opti-9 | 2.05 | 1.76 | 1.56 | N/A | 21 |
| Opti-10 | 2.69 | 3.04 | 2.39 | 2.60 | 22 |
| FXN | 1.00 | 1.00 | 1.00 | 1.00 | 93 |

### Example 12. Non-Human Primate Delivery Study

Non-human primates (NHPs) (n=4 Cynomolgus and n=4 Rhesus) were administered an AAV particle, serotype rh.10, containing one of the DNA constructs described in Table 24, formulated in phosphate buffered saline (PBS) with 0.001% F-68 (pluronic acid), via bolus or continuous intrathecal (IT) delivery using implanted catheters at cervical, thoracic and/or lumbar levels.

TABLE 24
Delivery Study in Non-Human Primates -
AAV Genome Sequences

| | Promoter | AAV | SEQ |
|---|---|---|---|
| Promoter | Position | Genome | ID NO |
| CBA (AKA CB6) | 535-794 | Self-Complementary | 91 |
| CB6 | 602-861 | Single-Stranded | 93 |
| CB6 | 472-736 | Self-Complementary | 94 |

The study design is shown in Table 25. Groups A and D were administered sequential 1 hour infusions at C1, T1 and L1 via a syringe pump (NE-300) at 1.5 ml/hour. Group B was administered a 10 hour infusion at L1 via an external CADD-Micro pump at 1.0 ml/hour and the NHPs were ambulatory wearing a jacket containing the pump. Group C was administered 3 bolus injections at L1 with 1 hour inter-dose intervals.

For Groups A, C and D, the NHPs were in a restrained prone position during administration. For Group D, the NHPs were returned to their cage between injections.

After the administration of the AAV construct, 200-500 ul of excipient (PBS with 0.001% F-68) was used to flush the catheter.

TABLE 25
Delivery Study in Non-Human Primates - Study Design

| | | AAV | | SEQ | | | | Conc. | Dose | N |
|---|---|---|---|---|---|---|---|---|---|---|
| Group | Capsid | Genome | Payload | ID NO | Site(s) | Rate | Vol. | (vg/ml) | (vg) | Species |
| A | AAVrh10 | SC | FXN | 91 | C1, T1, | 1.5 ml/h | 3 ml | 1 × 10¹³ | 3 × 10¹³ | 2 |
| | | | | | L1 | | | | | Rhesus |
| B | AAVrh10 | SC | FXN | 91 | L1 | 1.0 ml/h | 10 ml | 0.3 × 10¹³ | 3 × 10¹³ | 2 |
| | | | | | | | | | | Cynomolgus |
| C | AAVrh10 | SS | FXN | 93 | L1 | 3x Bolus | 3 ml | 1 × 10¹³ | 3 × 10¹³ | 2 |
| | | | | | | | | | | Cynomolgus |
| D | AAVrh10 | SC | GFP | 94 | C1, T1, | 1.5 ml/h | 3 ml | 1 × 10¹³ | 3 × 10¹³ | 2 |
| | | | | | L1 | | | | | Rhesus |

The NHPs were observed twice daily and the weights were taken weekly. Clinical pathology was done on Days 1, 3 and 29, serum antibodies were collected at day 1 and at day 29, CSF was collected prior to the study and at Day 0, 3 and 29. Three (3) weeks (for Group D) or 4 weeks (for Groups A, B and C) after AAV administration a saline perfusion was performed and the brain, spinal cord, dorsal root ganglia (DRGs), liver, spleen, heart, kidney, pancreas, ovaries and the sural nerve were collected as well as a skin biopsy.

Spinal cord cross sections were taken at cervical, thoracic and lumbar levels, and the segments were sliced into 2-3 mm thick cross sections. DRGs were collected at cervical, thoracic, lumbar and sacral levels, and the nerve roots were trimmed away prior to processing. Two mm diameter tissue punches from 3 mm thick coronal brain slices were taken from the pre-frontal cortex, motor cortex, putamen, dentate nucleus and cerebellar cortex. The level of frataxin mRNA was measured using a TaqMan two-steps real time quantitative PCR (RT-qPCR) assay. RNA extraction was performed on the tissue samples using Rneasy Plus Universal kit (Qiagen 73404). 1 (One) μg of total RNA was then reverse transcribed into single-stranded cDNA using High-Capacity cDNA Reverse Transcription Kit (Life Technology 4368813). The TaqMan gene expression assay specific for human frataxin (Hs00175940_m1) was carried in combination with either GAPDH (Rh02621745_g1), or Actin (Hs01060665_g1) in a 20 ul reaction volume.

Assays were performed in duplicate by two experimenters using a Lightcycler 480 II instrument (Roche). The amount of human frataxin mRNA for each sample was normalized to GAPDH or beta-Actin and further normalized relative to a control human brain sample. Each plate contained a No Template Control, a No Reverse Transcriptase control, a serially diluted human RNA and a serially diluted plasmid construct containing human frataxin. Amplification efficiency was determined for target and reference genes.

The relative quantity of human frataxin mRNA was averaged from four replicates and calculated according to the delta-delta Ct method (Livak and Schmittgen, 2001). The same procedure was performed to quantify eGFP mRNA using a TaqMan gene expression assay specific for eGFP (Mr04329676_mr). For eGFP, normalized Ct ratios were compared. Results for the 2 NHPs in Group A are shown in Table 26.

In Table 26, “BD” indicates that the detection of frataxin is below the lower limit of quantification of the assay. In Table 26, samples are identified using vertebral segmentation annotation.

TABLE 26
Delivery Study in Non-Human Primates, Group A - Frataxin expression
after sequential 1 hour IT infusions at C1, T1 and L1

| | | Fold Change Relative to Human Brain | |
|---|---|---|---|
| | | Frataxin Expression | |
| Sample | | NHP A1 | NHP A2 |
| Brain | Prefrontal Cortex | 4.79 | 0.05 |
| | Motor Cortex | 3.71 | BD |
| | Putamen | BD | BD |
| | Dentate Nucleus | BD | BD |
| | Cerebellar Cortex | 0.85 | 1.34 |
| Spinal Cord | C1 | 3.39 | 5.71 |
| | C3 | 3.89 | 1.91 |
| | C5 | 1.91 | 0.95 |
| | C7 | 5.44 | 0.42 |
| | T2 | 5.75 | 0.77 |
| | T4 | 7.23 | 0.92 |
| | T7 | 7.81 | 0.80 |
| | T8 | 25.39 | 2.49 |
| | T10 | 18.19 | 0.54 |
| | T12 | 21.75 | 1.91 |
| | L2 | 60.07 | 17.78 |
| DRG | C3 | 109 | 1.89 |
| | C5 | 96.67 | 1.50 |
| | T4 | 87.16 | 0.74 |
| | T7 | 15.22 | 1.23 |
| | L2 | 3.86 | 17.67 |
| | L6 | 121.10 | 13.04 |
| | S3 | 23.45 | 23.64 |

| | | |
|---|---|---|
| Liver | 156.90 | 256.20 |
| Spleen | 24.72 | 4.80 |

Frataxin expression was achieved in the pre-fontal, motor and cerebellar cortices but not the dentate nucleus. The 3-site IT infusion at C1, T1 and L1 resulted in a 31- to 42-fold spread of expression across the spinal cord. The DRGs generally showed expression greater than or equivalent to the spinal cord (with a 31- to 32-fold spread) and there was significant expression detected in the liver and spleen.

NHP A1 had a gradient of expression with the highest RNA levels in the lumbar region that were ˜30× level in human brain. Expression in the cervical region was 2-3-fold normal.

NHP A2, while showing lower expression than NHP A1, again had the highest expression (˜10-fold) in the lumbar region. The thoracic cord had the lowest level of expression, which was up to 2-fold normal.

DRG expression was similar to the spinal cord with much higher levels in NHP A1 than NHP A2. FXN expression was 10-100 fold higher than human brain levels in this f animal. NHP A2 had lower FXN expression with cervical and thoracic DRGs at levels comparable to the human brain, however the lumbar and sacral DRGs had higher levels, suggesting a preferential transduction of these lower DRGs despite infusions at multiple levels along the cord.

Results for the 2 NHPs in Group B are shown in Table 27.

In Table 27, “BD” indicates that the detection of frataxin is below the lower limit of quantification of the assay. In Table 27, samples are identified using vertebral segmentation annotation.

TABLE 27
Delivery Study in Non-Human Primates, Group B - Frataxin
expression after 10 hour IT infusion at L1

| | | Fold Change Relative to Human Brain | |
|---|---|---|---|
| | | Frataxin Expression | |
| Sample | | NHP B1 | NHP B2 |
| Brain | Prefrontal Cortex | 0.33 | BD |
| | Motor Cortex | 0.92 | BD |
| | Putamen | BD | BD |
| | Dentate Nucleus | 0.10 | 0.08 |
| | Cerebellar Cortex | 0.95 | 1.56 |
| Spinal Cord | C1 | 2.26 | 0.27 |
| | C3 | 2.43 | 1.06 |
| | C5 | 3.00 | 0.95 |
| | C7 | 5.24 | 2.20 |
| | T2 | 6.46 | 1.21 |
| | T4 | 10.31 | 7.47 |
| | T7 | 11.99 | 0.90 |
| | T8 | 15.85 | 3.61 |
| | T10 | 20.31 | 1.42 |
| | T12 | 12.23 | 2.82 |
| | L2 | 10.04 | 2.82 |
| DRG | C3 | 0.62 | 2.03 |
| | C5 | 1.00 | 2.82 |
| | T4 | 67.28 | 24.71 |
| | T7 | 0.73 | 1.13 |
| | L2 | 4.38 | 5.19 |
| | L6 | 0.46 | 19.51 |
| | S3 | 42.80 | 28.77 |

| | | |
|---|---|---|
| Liver | 416.70 | 729.00 |
| Spleen | 5.51 | 5.00 |

Frataxin expression was achieved in the pre-fontal, motor and cerebellar cortices with significant expression in the dentate nucleus. The 10 hour IT infusion at L1 resulted in a 9 to 28-fold spread of expression across the spinal cord with higher levels at thoracic and lumbar levels.

NHP B1 showed gradient of expression with highest levels (10-20 fold human brain) in the lower thoracic region rostral to the site of infusion and lower levels (˜2 fold normal) in the cervical spinal cord. NHP B2 also had a gradient of FXN expression with higher levels in the lower thoracic region and less FXN in the cervical spinal cord.

The DRGs generally showed expression greater than or equivalent to the spinal cord (with a 25- to 146-fold spread) and significant expression was detected in the liver and spleen.

Results for the 2 NHPs in Group C are shown in Table 28.

In Table 28, “BD” indicates that the detection of frataxin is below the lower limit of quantification of the assay. In Table 28, samples are identified using vertebral segmentation annotation.

TABLE 28
Delivery Study in Non-Human Primates, Group C - Frataxin
expression after IT triple bolus injection at L1

| | | Fold Change Relative to Human Brain | |
|---|---|---|---|
| | | Frataxin Expression | |
| Sample | | NHP C1 | NHP C2 |
| Brain | Prefrontal Cortex | BD | BD |
| | Motor Cortex | BD | BD |
| | Putamen | BD | BD |
| | Dentate Nucleus | 0.04 | BD |
| | Cerebellar Cortex | 0.13 | 0.11 |
| Spinal Cord | C1 | 0.78 | 0.12 |
| | C3 | 0.57 | 0.30 |
| | C5 | 0.47 | 5.51 |
| | C7 | 13.32 | 0.62 |
| | T2 | 0.71 | 0.91 |
| | T4 | 1.79 | 2.22 |
| | T7 | 2.12 | 1.11 |
| | T8 | 3.32 | 1.28 |
| | T10 | 2.38 | 1.46 |
| | T12 | 2.87 | 2.51 |
| | L2 | 4.05 | 2.66 |
| DRG | C3 | 0.15 | 0.05 |
| | C5 | 2.87 | BD |
| | T4 | 0.16 | 0.10 |
| | T7 | BD | 0.07 |
| | L2 | 0.25 | 0.21 |
| | L6 | 40.71 | 17.20 |
| | S3 | 0.31 | 4.09 |

| | | |
|---|---|---|
| Liver | 1.19 | 4.53 |
| Spleen | 0.5 | 0.35 |

Comparison of prolonged and bolus delivery approaches demonstrated that prolonged infusion yielded more homogenous levels of protein expression across the spinal cord vs. bolus dosing at one or multiple levels. There was less variability with prolonged infusion across the spinal cord. Such prolonged infusion showed no more than 2-fold variability whereas bolus infusion exhibited over 4-fold differences across the spinal cord. For the prolonged infusion, the highest spinal cord expression was observed just above (rostral) to the tip of the infusion catheter. The DRGs generally had lower expression than with multi-site infusion. In the multi-site infusion, a caudal-rostral gradient in the spinal cord was observed, despite cervical, thoracic and lumbar infusion sites. DRG expression was very high across all levels. With the lumbar bolus, a caudal-to-rostral gradient of expression in the spinal cord with variable expression in DRGs was observed.

Low levels of frataxin expression were seen in the cerebellar cortex with very low or no expression seen in the pre-frontal and motor cortices, putamen and dentate nucleus. The triple IT bolus at L1 yielded a rostral-caudal expression pattern with the highest levels seen at the lower thoracic and lumbar levels and spikes in the lower cervical region with a 28- to 46-fold spread.

The DRG expression showed greater than 100-fold spread with no detectable expression in isolated cervical/thoracic DRGs. Moderate levels of expression were seen in the liver and spleen.

As shown in Table 29, the high expresser in Group D, the GFP control, showed a transgene (GFP) mRNA expression pattern across the spinal cord that was similar to the transgene (human frataxin) mRNA expression pattern in the high expresser in Group A (NHP A1). In Table 29, samples are identified using vertebral segmentation annotation.

TABLE 29
Delivery Study in Non-Human Primates - High
Expressers in Group A and Group D after Sequential
1 hour IT infusions at C1, T1 and L1

| | |
|---|---|
| Spinal Cord | Fold Transgene Expression Relative to beta-actin |

| Segment | NHP A1 (Group A) | NHP D1 (Group D) |
|---|---|---|
| C1 | 0.03 | 0.05 |
| C5 | 0.02 | 0.08 |
| C7 | 0.05 | 0.13 |
| T2 | 0.05 | 0.14 |
| T7 | 0.07 | 0.10 |
| T10 | 0.16 | 0.15 |
| T12 | 0.19 | 0.15 |

### Example 13. Comparison of Human FXN Expression Following Intrastriatal Delivery in Mice of AAV Constructs Containing Three Different Promoters

To compare human FXN expression driven by PGK, CMV and CBA and FXN promoters, more than 93 wild type mice (C57BL/6), 6-8 weeks old, were administered an AAV with dose levels shown in Tables 30-32. The AAVs were formulated in PBS and 0.001% F-68, and 5 uL administered via intrastriatal (IS) injection. Human FXN (hFXN) protein levels in striatum were quantified after 7 days (Tables 30 and 31) or 28 days (Table 32) by ELISA with an assay (Abcam) specific for human FXN (no detection of mouse FXN).

Seven days after AAV intrastriatal administration (5E9 VG), all AAV constructs resulted in human frataxin expression. The CBA promoter drove the highest expression, followed by PGK and CMV promoters. The same rank order of promoter-driven expression (CBA>PGK>CMV) was observed with constructs expressing wild-type human frataxin (Table 30) and codon optimized human frataxin (Table 31) at 7 days post-administration. With the CBA promoter, wild-type frataxin (scAAVrh10-CBA-FXN) and codon-optimized frataxin (scAAVrh10-CBA-Opti10FXN) resulted in similar levels of human frataxin protein in the striatum at this time point (Table 31).

TABLE 30
Striatum Levels of human FXN at 7 days Following Intrastriatal
Injection of Wild-Type Frataxin Constructs

| | AAV | Dose | Inj. Site |
|---|---|---|---|
| Test Article | Genome | (VG) | AVG ± SEM |
| scAAVrh10-CBA-FXN | SC | 5 × 10⁹ | 120.1 ± 20.18 |
| scAAVrh10-CMV-FXN | SC | 5 × 10⁹ | 31.20 ± 12.18 |
| scAAVrh10-PGK-FXN | SC | 5 × 10⁹ | 55.49 ± 4.69 |
| Vehicle | — | — | 2.35 ± 1.33 |

TABLE 31
Striatum Levels of human FXN at 7 days Following Intrastriatal
Injection of Codon-Optimized Frataxin (Opti10FXN) Constructs

| | AAV | Dose | Inj. Site |
|---|---|---|---|
| Test Article | Genome | (VG) | AVG ± SEM |
| scAAVrh10-CBA-Opti10FXN | SC | 5 × 10⁹ | 64.12 ± 15.45 |
| scAAVrh10-CMV-Opti10FXN | SC | 5 × 10⁹ | 18.85 ± 3.93 |
| scAAVrh10-PGK-Opti10FXN | SC | 5 × 10⁹ | 29.96 ± 6.16 |
| scAAVrh10-CBA-FXN | SC | 5 × 10⁹ | 61.89 ± 3.77 |
| Vehicle | — | — | 2.00 ± 0.09 |

Twenty-eight days after AAV intrastriatal administration (5E8, 5E9 or 5E10 VG), all AAV constructs resulted in human frataxin expression in the striatum (Table 32). For all 3 promoters (CBA, CMV, PGK), each log increase in dose resulted in an approximately 6-8 fold increase in human frataxin protein levels in the striatum. The CBA promoter drives the highest level of expression in the striatum, followed by the CMV and PGK promoters. The rank order of promoter-driven expression (CBA>CMV>PGK) was observed at 28 days post-administration, with the CBA promoter resulting in approximately 3-fold higher levels of human FXN protein expression than the CMV promoter, and the CMV promoter resulting in approximately 3-fold higher levels of human FXN protein expression than the PGK promoter, across the dose levels used.

TABLE 32
Striatum Levels of human FXN at 28 days Following Intrastriatal
Injection of Wild-Type and Codon-Optimized Frataxin Constructs

| | AAV | Dose | Inj. Site |
|---|---|---|---|
| Test Article | Genome | (VG) | AVG ± SEM |
| scAAVrh10-CBA-FXN | SC | 5 × 10⁸ | 113.51 ± 10.32 |
| scAAVrh10-CBA-FXN | SC | 5 × 10⁹ | 748.53 ± 120.54 |
| scAAVrh10-CBA-FXN | SC | 5 × 10¹⁰ | 4915.25 ± 896.59 |
| scAAVrh10-CMV-FXN | SC | 5 × 10⁸ | 33.37 ± 4.91 |
| scAAVrh10-CMV-FXN | SC | 5 × 10⁹ | 260.67 ± 12.61 |
| scAAVrh10-CMV-FXN | SC | 5 × 10¹⁰ | 1687.10 ± 278.23 |
| scAAVrh10-PGK-FXN | SC | 5 × 10⁸ | 12.49 ± 1.02 |
| scAAVrh10-PGK-FXN | SC | 5 × 10⁹ | 79.93 ± 1.60 |
| scAAVrh10-PGK-FXN | SC | 5 × 10¹⁰ | 515.81 ± 29.32 |
| scAAVrh10-CBA-Opti10FXN | SC | 5 × 10⁹ | 777.94 ± 176.08 |
| Vehicle | | | 5.47 ± 2.76 |

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1-2. (canceled)

3. The polynucleotide of claim 7, wherein the polynucleotide comprises at least one 5′ inverted terminal repeat (ITR), one 3′ inverted terminal repeat ITR, a promoter region, and an enhancer, and wherein said enhancer is a CMV enhancer.

4. The polynucleotide of claim 3, wherein the open reading frame encoding human frataxin is codon optimized.

5-6. (canceled)

7. A polynucleotide encoding human frataxin comprising a sequence having at least 95% identity to a sequence selected from the group consisting of SEQ ID NOs 1-94 or variants thereof.

8. The polynucleotide of claim 7, comprising a sequence having at least 99% identity to a sequence selected from the group consisting of SEQ ID NOs 1-94 or variants thereof.

9. A polynucleotide comprising a sequence selected from the group consisting of SEQ ID NOs 1-94, wherein the frataxin payload is replaced by gene encoding a CNS protein of interest or a modulatory nucleic acid.

10. The polynucleotide of claim 9, wherein the polynucleotide has at least 95% identity to a sequence selected from the group consisting of SEQ ID NOs 1-94 or variants thereof.

11. (canceled)

12. An AAV particle comprising the polynucleotide of claim 7 packaged in a capsid, said capsid having a serotype selected from the group consisting of AAV1, AAV2, AAV3, AAV4, AAV5, AAV6, AAV7, AAV8, AAV9, AAV9.47, AAV9(hu14), AAV10, AAV11, AAV12, AAVrh8, AAVrh10, AAV-DJ and AAVDJ8.

13-18. (canceled)

19. The method of claim 18, wherein the polynucleotide in packaged in an AAV particle, said AAV particle comprising a capsid having a capsid serotype selected from the group consisting of AAV1, AAV2, AAV3, AAV4, AAV5, AAV6, AAV7, AAV8, AAV9, AAV9.47, AAV9(hu14), AAV10, AAV11, AAV12, AAVrh8, AAVrh10, AAV-DJ and AAVDJ8.

20-24. (canceled)

25. The method of claim 26, wherein increasing the level of protein treats a disease or disorder, wherein the disease or disorder is Friedreich's Ataxia.

26. A method of increasing the level of a protein in the CNS of a subject in need thereof comprising administering to said subject via prolonged infusion an effective amount of an AAV particle comprising a viral genome encoding a sequence selected from the group consisting of SEQ ID NOs 1-94 or a therapeutically effective amount of a polynucleotide comprising a sequence selected from the group consisting of SEQ ID NOs 1-94 wherein the frataxin payload has been replaced by gene encoding a CNS protein of interest or a modulatory nucleic acid.

27. The method of claim 26, wherein the prolonged infusion is by intracerebroventricular (ICV) administration.

28. The method of claim 26, wherein the prolonged infusion is by injection into the ventricular system of the brain.

29. The method of claim 28, wherein the injection to the ventricular system occurs in at least one location selected from the group consisting of the right lateral ventricle, left lateral ventricle, third ventricle, fourth ventricle, interventricular foramina (also called the foramina of Monro), cerebral aqueduct, central canal, median aperture, right lateral aperture, and left lateral aperture, perivascular space.

30-32. (canceled)

33. The method of claim 26, wherein the prolonged infusion is by intrathecal (IT) administration.

34. The method of claim 33, wherein IT administration occurs by injection into the subarachnoid space, and wherein the injection into the subarachnoid space occurs in at least one location selected from the group consisting of the cervical, thoracic, lumbar and sacral regions of the spine, wherein if the injection occurs in the cervical region the injection occurs in a location selected from the group consisting of C1, C2, C3, C4, C5, C6, and C7, wherein if the injection occurs in the thoracic region the injection occurs in a location selected from the group consisting of T1, T2, T3, T3, T4, T5, T6, T7, T8, T9, T10, T11, and T12, wherein if the injection occurs in the lumbar region the injection occurs in a location selected from the group consisting of L1, L2, L3, L4, and L5, and wherein if the injection occurs in the sacral region the injection occurs in a location selected from the group consisting of S1, S2, S3, S4 and S5.

35-41. (canceled)

42. The method of claim 26 wherein the prolonged infusion occurs at a volume selected from the group consisting of, a volume of more than 1 mL, a volume of less than 1 mL, a volume between about 0.1 mL to about 120 mL, a volume of at least 3 mL, a volume of 3 mL, a volume of at least 10 mL, and a volume of 10 mL.

43-48. (canceled)

49. The method of claim 26, wherein the prolonged infusion occurs for at least a duration selection from the group consisting of 0.17, 0.33, 0.5, 0.67, 0.83, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, and 36 hour(s).

50-53. (canceled)

54. The method of claim 26, wherein the prolonged infusion occurs at a constant rate.

55. The method of claim 26, wherein the prolonged infusion occurs at a ramped rate.

56. The method of claim 55, wherein the ramped rate increases over the duration of the prolonged infusion.

57. The method of claim 26, wherein the prolonged infusion occurs at a complex rate alternating between high and low rates over the duration of the prolonged infusion.

58. (canceled)

59. The method of claim 26, wherein prolonged infusion occurs at a rate between about 0.1 mL/hour and about 25.0 mL/hour, wherein the rate of prolonged infusion is selected from the group consisting of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, and 25.0 mL/hour.

60-62. (canceled)

63. The method of claim 26, wherein administration comprises a total dose between about 1×10⁶ VG and about 1×10¹⁶ VG, wherein the total dose is selected from the group consisting of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, and 1×10¹⁶ VG.

64-67. (canceled)

68. The method of claim 26, wherein the prolonged infusion comprises a composition concentration between about 1×10⁶ VG/mL and about 1×10¹⁶ VG/mL of AAV comprising at least one polynucleotide encoding a payload, wherein the concentration is selected from the group consisting of 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, and 1×10¹⁶ VG/mL.

69-78. (canceled)

79. The method of claim 26, further comprising co-administration of intravenous mannitol.

80. (canceled)

81. The method of claim 79, wherein a dose of approximately 0.25 to 1.0 g/kg intravenous mannitol is co-administered, and wherein the dose of intravenous mannitol is selected from the group consisting of 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, and 1.00 g/kg.

82-88. (canceled)

89. The method of claim 26 wherein the AAV comprises a capsid selected from the group consisting of AAV1, AAV2, AAV3, AAV4, AAV5, AAV6, AAV7, AAV8, AAV9, AAV10, AAV11, AAV12, AAVrh8, AAVrh10, AAV-DJ, and AAV-DJ8.

90. The method of claim 89, wherein the AAV comprises a self-complementary (SC) genome.

91. The method of claim 89, wherein the AAV comprises a single stranded (SS) genome.

92-93. (canceled)

94. The method of claim 26, wherein the subject is in a position selected from the group consisting of horizontal for the duration of the prolonged infusion, vertical for the duration of the prolonged infusion, at an angle between approximately horizontal 0° to about vertical 90° for the duration of the prolonged infusion, the position of the subject changes during the duration of the prolonged infusion from horizontal to vertical, and the position of the subject changes during the duration of the prolonged infusion from vertical to horizontal.

95-101. (canceled)
